SEQUENCE LISTING (123 sequences)

SEQ ID NO: 1 - protein, 626 aa, Artificial Sequence
  NR4A3 Isoform Alpha
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 2 - protein, 443 aa, Artificial Sequence
  NR4A3 Isoform Beta
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRVSFMISCFQMNDQGLYLWLLVIRVD

SEQ ID NO: 3 - protein, 637 aa, Artificial Sequence
  NR4A3 Isoform 3
MHDSIRFGNVDMPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 4 - protein, 331 aa, Artificial Sequence
  Wild-type human c-Jun
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 5 - DNA, 3257 bp, Artificial Sequence
  Wild-type human c-Jun
gctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagccaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcacgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccacagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaa

SEQ ID NO: 6 - DNA, 993 bp, Artificial Sequence
  Wild-type human c-Jun (coding region)
atgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattt

SEQ ID NO: 7 - DNA, 993 bp, Artificial Sequence
  c-Jun codon optimized #1
atgacagccaagatggaaaccacattctacgacgacgccctgaacgcctcattcctgccttctgagagcggaccttacggctacagcaatcctaagatcctgaaacagagcatgacccttaacctggctgatcctgttggaagcctgaaacctcacctgagagccaaaaacagcgacctgctcaccagccctgatgtgggcctgctgaagctggcctctccagagctggaacggctgatcatccagagcagcaacggccacatcacaaccacccctacccctacacaattcctgtgccctaagaacgtgaccgacgagcaggagggcttcgccgaaggctttgtgcgggccctggcagaactgcactctcagaacaccctgcctagcgtgacctccgccgcccagcctgtcaacggcgccggaatggtggcccctgccgtggcttctgtggccggcggcagcggcagcggcggattcagcgcctctctgcactctgagcctcctgtctacgccaatctgtctaatttcaaccccggagccctgtccagcggcggcggagctcctagctacggcgctgctggactggccttccccgcccagccccagcaacagcagcagcctccacaccacctgccccagcagatgcccgtgcagcaccctagactgcaggccctgaaggaagaaccccaaacagtgcctgagatgcctggcgagacacctccactgagccccatcgacatggaaagccaggagcggatcaaggccgagagaaagagaatgcggaacagaatcgccgctagcaagtgcagaaagcggaagctggaaagaatcgccagactggaagagaaggtgaagaccctgaaagcccaaaatagcgagctggccagcaccgccaacatgctgcgggaacaggtggcccagctgaagcagaaggtgatgaaccacgtgaactctggttgtcagctgatgctgacccagcagctccagaccttc

SEQ ID NO: 8 - DNA, 993 bp, Artificial Sequence
  c-Jun codon optimized #2
atgacagccaagatggaaaccaccttctacgacgacgccctcaacgcctccttcctgccttctgagagcggtccttacggctacagcaaccccaagatcctgaagcaaagcatgaccctgaacctggccgaccccgttggctccctgaaacctcacctgagagccaaaaacagcgacctgctgaccagccctgatgtgggcctgctgaagctggcctctccagagctggaaagactgattatccagagcagcaacggccacatcaccacaacacctacccctacacagttcctgtgccctaagaacgtgactgatgagcaggagggctttgccgagggcttcgtgagagccctggctgagctgcattctcagaacaccctgcctagcgtgacctctgccgcccagcctgttaatggcgccggcatggtggcccctgccgtggcctctgtggccggaggcagcggcagcggcggattcagcgcctctctgcacagcgagccccccgtctacgccaacctgagcaatttcaaccctggcgccctgtccagcggcggcggcgccccttcatatggcgctgccggcctggccttccccgctcagccccagcagcagcaacagcctccacaccacctgccccagcagatgcccgtgcagcaccccagactgcaggccctgaaggaagaacctcagaccgtgcccgagatgcctggcgagacccctcctctgagccctatcgacatggaaagccaggagagaatcaaggccgagaggaagcggatgcggaacagaatcgccgccagcaagtgcagaaaaagaaagctggaacggatcgccagactggaggagaaggtgaagacactgaaagcccaaaattctgaactggcctctaccgccaatatgctgcgcgagcaggtggctcaactgaagcagaaggtgatgaaccacgtgaacagcggatgtcagctgatgctgacacagcagctgcagactttt

SEQ ID NO: 9 - DNA, 993 bp, Artificial Sequence
  c-Jun codon optimized #3
atgaccgccaagatggaaaccaccttctacgacgacgccctgaacgccagctttctgccttctgagtctggcccctacggctacagcaaccccaagatcctgaagcagagcatgaccctgaacctggccgatcctgtgggcagcctgaaacctcacctgagagccaagaacagcgacctgctgacaagccctgatgtgggcctgctgaaactggcctctcctgagctggaacggctgatcatccagagcagcaacggccacatcaccaccacacctacaccaacacagtttctgtgccccaagaacgtgaccgacgagcaagagggattcgccgagggctttgttagagccctggccgaactgcacagccagaataccctgcctagcgtgacatctgccgctcagcctgttaatggcgccggaatggttgctcctgccgtggcttctgttgctggcggatctggatctggcggctttagcgcctctctgcactctgagcctccagtgtacgccaacctgagcaacttcaaccctggcgctcttagctctggtggcggagcaccttcttatggcgctgccggattggcctttcctgctcagcctcagcagcagcaacagcctcctcatcatctgccccagcagatgcctgtgcagcaccctagactgcaggccctgaaagaggaaccccagacagtccctgagatgcccggcgaaacacctcctctgagccccatcgacatggaaagccaagagcggatcaaggccgagcggaagcggatgagaaatagaatcgccgcctccaagtgccggaagaggaagctggaaagaatcgcccggctggaagagaaagtgaaaaccctgaaggcccagaactccgagctggcctctaccgccaacatgctgagagaacaggtggcccagctgaaacagaaagtcatgaaccacgtgaacagcggctgccagctgatgctgacacagcagctgcagaccttc

SEQ ID NO: 10 - DNA, 993 bp, Artificial Sequence
  c-Jun codon optimized #4
atgactgccaaaatggagactacattctatgacgacgccctcaatgccagttttttgccgagtgaatccggcccctacggctattcaaaccctaagatcctcaagcaatcaatgaccctcaatcttgctgacccagttggctccctgaaaccccatctcagagctaaaaatagtgacctccttacttcccctgatgttggactcctcaaacttgcttctcccgaactcgaacgcttgatcattcaatcttccaacggccacatcacaacaacacccacacccacccagtttctttgcccaaaaaatgtcaccgatgaacaggaaggtttcgcggaaggattcgtccgcgcgctggccgaactgcactcccagaatacacttccttcagttacgtcagccgcccagccagtgaatggtgcgggaatggttgctcctgcggtcgcttctgtcgcagggggctccggttctggcggatttagcgcctctctgcattccgagccacctgtatatgctaatctttctaattttaaccccggagccttgtctagcggcggtggtgcccccagctacggtgctgcaggactcgccttcccagctcaacctcagcagcagcaacaacccccccatcaccttccccaacagatgccagtacaacatccaaggctccaggccctcaaagaggaaccacagacggtgcccgaaatgcctggcgaaactccaccactttcccctattgatatggaatcccaagagcgcatcaaggccgaaagaaagcgaatgcggaatagaatagcagcttcaaaatgtagaaaacggaaattggaacgaatcgcacggttggaagaaaaggtgaagaccttgaaagcccagaacagtgagctcgcctctaccgctaacatgctgcgcgagcaagtcgcacaacttaagcagaaggtgatgaaccatgtgaatagcggatgtcaacttatgctgactcaacagttgcaaaccttt

SEQ ID NO: 11 - DNA, 993 bp, Artificial Sequence
  c-Jun codon optimized #5
atgaccgcgaaaatggagacaacattttacgatgatgcactgaacgcctcttttctgccaagtgaatccggcccctacggatactcaaaccctaagattctgaaacagtctatgactctcaacctggccgacccagttggcagtctgaagcctcatttgcgagccaagaatagtgatctgctgacctccccagacgtgggactgctgaaactcgcctcacctgaacttgagcgcttgattatacagtcatccaatgggcacatcacaacaacacctactcctacccagtttctgtgccccaaaaacgtcaccgatgagcaggagggattcgcggaaggctttgtgcgcgccctggctgaattgcatagtcagaacactcttcccagcgtaaccagcgccgcccaaccagtgaatggagccggtatggtggctcccgcggtggctagtgttgcgggggggtcaggctctggtgggttcagtgcttctcttcactctgaaccccctgtgtatgccaatctgtctaactttaaccctggggccctctcctctggtgggggtgcccccagctacggagcggccggcctggcctttcctgcccagcctcagcagcagcagcaaccccctcatcatcttccgcagcagatgccagtacagcatccacgcctgcaggctcttaaggaggagccccagacggtgcccgaaatgcccggggaaactccacccttgtcccccattgacatggagtcccaggagcggatcaaggctgaaagaaagaggatgcggaatcgcatcgcagcctctaaatgccgcaagcggaaacttgagaggatcgcgcggttggaggaaaaagtaaaaaccttgaaggcacagaactctgagctggcgagtactgccaacatgctcagagaacaagtcgcacagctgaagcagaaagtgatgaaccatgtgaacagcggttgtcagctgatgctgactcagcagctgcagaccttc

SEQ ID NO: 12 - DNA, 993 bp, Artificial Sequence
  c-Jun codon optimized #6
atgaccgccaagatggagaccacattctacgatgacgctctgaacgcttcctttctgccttccgagtccggcccctacggctactccaatcccaagattctgaagcagagcatgacactgaatctggctgatcccgtgggatctctgaagcctcatctgagagccaagaattccgatctgctgacaagccccgacgtgggactgctcaaactggccagccccgaactggagaggctcattatccagagctccaacggccacatcaccacaacacctacccctacccagtttctctgtcccaagaacgtgacagacgagcaagagggatttgccgaaggcttcgtgagagccctcgccgaactgcatagccagaacacactgccttccgtgaccagcgctgctcaacccgtgaacggcgctggcatggtcgctcccgccgtcgccagcgtggctggaggaagcggatccggaggcttcagcgcttccctccacagcgaacctcccgtgtacgctaatctgagcaacttcaaccccggcgctctgagcagcggaggaggagctcctagctatggagctgccggactggcttttcccgcccagccccagcagcagcagcagcccccccatcatctgcctcagcagatgcccgtgcagcatcccagactccaagctctgaaggaggagcctcagaccgtccccgagatgcccggcgaaaccccccctctgtcccccatcgacatggaaagccaagagaggatcaaggccgagaggaagaggatgaggaatagaatcgccgccagcaagtgtagaaagaggaagctggagaggatcgccagactggaggagaaggtgaagaccctcaaggctcagaattccgagctggccagcacagccaacatgctgagagagcaagtggcccagctcaagcagaaggtgatgaaccacgtcaacagcggatgccagctgatgctcacccagcagctgcagaccttc

SEQ ID NO: 13 - DNA, 993 bp, Artificial Sequence
  c-Jun codon optimized #7
atgaccgctaaaatggaaaccactttctatgacgatgccctgaacgcctccttccttccgtccgagtccggaccctacggatactcaaatcctaagatcctcaaacagtcgatgaccctcaacctggccgaccccgtgggatccctgaagccgcacttgcgcgccaagaactccgacctcctgacgagcccagacgtgggcctgctgaagctcgcatcacccgaacttgagcggttgatcattcagtcctccaacggacatatcaccaccactcccaccccaactcagtttctgtgtccgaagaacgtgaccgatgagcaagagggattcgccgagggattcgtgcgggccctggccgagctgcatagccagaacacccttccatccgtgacctcggcggctcagcctgtgaacggcgcgggaatggtcgcgcccgccgtggcctcggtggccgggggcagcggcagcgggggattttccgcgtcgctgcactccgagccgccggtgtacgccaacctgtcaaacttcaaccctggggccctgagctccggcggtggagcaccttcgtacggcgccgctggcctggcgttccccgcgcaaccacagcagcaacagcagccccctcaccacctcccccaacaaatgcctgtgcagcacccgaggctgcaggccctcaaggaagaaccccagactgtgccggaaatgccgggggagactccgccgctgtcccctatcgacatggaatcacaggaacgcattaaggcagagcggaagcgcatgcggaaccggattgccgcctccaagtgccgcaagagaaagctcgaaagaatcgccagattggaagaaaaggtcaagactctgaaggcccagaactctgagctggcatccaccgctaatatgctgagggaacaagtggcccagctgaaacagaaggtcatgaaccacgtcaacagcggttgccagctgatgctgacccagcaactccagacattc

SEQ ID NO: 14 - DNA, 993 bp, Artificial Sequence
  c-Jun codon optimized #8
atgaccgccaagatggagaccaccttctacgacgacgccctgaacgccagcttcctgcccagcgagagcggaccctacggctactctaaccccaagatcctgaaacagagcatgacactgaatctggccgaccccgtgggcagcctgaagcctcaccttagagccaagaacagcgacctgctgaccagccccgacgtgggcctgctgaagctcgcctctccagagttagagagactgatcatccagtccagcaacggccacatcacaaccaccccaacccctacccagttcctgtgccccaagaacgtgaccgacgagcaggagggcttcgccgagggctttgtgagagccctggccgagttgcactctcagaacaccctgccctccgtgaccagcgccgctcaacctgtgaacggcgcaggaatggttgctcctgccgtggccagcgttgcaggcggatctggaagtggaggcttctccgcctcccttcacagcgagcctcccgtgtacgccaacctgagcaacttcaaccccggcgccctgagcagtggaggaggcgctcccagctatggagcagctggattagccttccccgcccagccacagcagcagcaacagcctccccaccacctgcctcagcaaatgcctgtgcagcaccctcggctgcaggcccttaaggaggagccccagaccgttcctgagatgcctggcgagacccctcccctgagccctatcgacatggagtcccaggagcggatcaaggccgagcggaagcggatgcggaaccggatcgctgcttccaagtgccggaagagaaagctggagagaatcgcccggctggaggagaaggtgaagaccctgaaggcccagaactccgagctggcctccaccgccaacatgctgcgggagcaggttgcacagctgaagcagaaggtcatgaaccacgtgaacagcggctgccagctgatgctgacccagcagctgcagaccttc

SEQ ID NO: 15 - DNA, 1059 bp, Artificial Sequence
  c-Jun codon optimized #9
atgacagcgaagatggagacaaccttctatgacgatgctcttaacgcctccttcctgccttccgaaagcgggccctacgggtactctaatcctaagatacttaagcaatcgatgactctcaacctcgctgacccggttggctcactgaaaccacacctgagagctaagaatagtgacctgctcactagtcccgatgtcgggcttctgaagctggcctctcccgagctggagaggcttatcatccaatcatcaaatggccacatcaccactaccccaacaccaactcaattcctttgccctaaaaacgtgaccgacgaacaggaaggcttcgccgagggttttgtccgggccttggccgagctgcattctcaaaatacactgccaagcgtcacttctgcggcgcagccggttaacggagcagggatggtggctcccgccgttgctagcgtggccggcggttccggctccggcggtttctctgcctccttgcattctgagccaccagtctacgcgaacctgtccaactttaatccgggggcgctgagtagcggaggcggcgcccctagctatggggcagctggactggccttcccggcacaaccccaacaacaacagcaaccgccacaccatcttcctcaacaaatgccagtgcaacatccacgcttacaagccctcaaggaggaaccccagaccgtgcctgagatgcccggcgaaaccccgccattgagccctattgacatggaaagtcaagagagaattaaggcagagcgcaagagaatgaggaaccggatcgcagcatctaagtgccgcaaacggaaattggagcggatcgctcgcttggaggagaaggtcaagactctcaaggcccagaactccgagcttgcgagcacagctaatatgctgcgcgagcaggtggcccagttaaaacaaaaggtcatgaaccatgtgaacagcggctgtcagctgatgcttacgcaacagctgcaaacctttggctccggtgcaacgaacttcagcctgctgaagcaggccggagatgttgaggaaaatccaggtccc

SEQ ID NO: 16 - DNA, 993 bp, Artificial Sequence
  c-Jun codon optimized #10
atgacggccaaaatggagactacgttctacgatgacgcactcaacgcgtccttcctgccctctgagagtggaccctatggctactccaatccaaagatcctgaagcagtctatgaccctcaacctggcggacccggtgggctcccttaagccgcacttgcgcgccaagaactccgacctgctgacctcccctgatgtgggcctcctcaagctcgctagccctgaattggagaggctgatcatccagagctcaaatggccacatcaccaccacacctaccccaacccagttcctgtgcccaaaaaacgtgaccgacgagcaggagggcttcgcggagggcttcgtcagagctctggccgagctgcactcacagaacacgctcccttccgtgacctccgctgcccagccggtcaatggcgctggaatggtggctccggctgtggcctctgttgccggcggctccggctccggaggcttttcagcttctctgcattctgagcccccagtgtacgctaacctgagcaacttcaaccccggggcgctcagctccggtggcggtgccccgagctacggcgcggctgggctggcgttccccgctcagcctcagcagcaacagcaacctccccaccacctgccacagcagatgcctgtgcagcacccacgcctgcaggccttgaaggaggaacctcagactgtgccagagatgcccggcgagaccccacccctgtccccgattgacatggagagccaggagcgcatcaaggcagagcgcaagcgtatgcgcaaccgcatcgcggcctccaagtgccgaaagcgcaagctggagcggattgctcgcctggaggagaaggtgaagaccctgaaggcccagaattccgagctggcctcgaccgccaacatgctacgagaacaggtcgcgcagctgaaacagaaggtcatgaaccatgtcaacagcgggtgccagctgatgttgacccagcagcttcagaccttc

SEQ ID NO: 17 - protein, 121 aa, Artificial Sequence
  R12 VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 18 - protein, 5 aa, Artificial Sequence
  R12 VH CDR1
AYYMS

SEQ ID NO: 19 - protein, 17 aa, Artificial Sequence
  R12 VH CDR2
TIYPSSGKTYYATWVNG

SEQ ID NO: 20 - protein, 12 aa, Artificial Sequence
  R12 VH CDR3
DSYADDGALFNI

SEQ ID NO: 21 - protein, 112 aa, Artificial Sequence
  R12 VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 22 - protein, 12 aa, Artificial Sequence
  R12 VL CDR1
TLSSAHKTDTID

SEQ ID NO: 23 - protein, 7 aa, Artificial Sequence
  R12 VL CDR2
GSYTKRP

SEQ ID NO: 24 - protein, 9 aa, Artificial Sequence
  R12 VL CDR3
GADYIGGYV

SEQ ID NO: 25 - RNA, 20 nt, Artificial Sequence
  NR4A1 sgRNA 5
gaaguccucgaacuugaagg

SEQ ID NO: 26 - RNA, 20 nt, Artificial Sequence
  NR4A1 sgRNA 6
accuucauggacggcuacac

SEQ ID NO: 27 - RNA, 20 nt, Artificial Sequence
  NR4A2 sgRNA 1
uugggauggucaaagaaggu

SEQ ID NO: 28 - RNA, 20 nt, Artificial Sequence
  NR4A2 sgRNA 2
cagccaggcacuucugaaau

SEQ ID NO: 29 - RNA, 20 nt, Artificial Sequence
  NR4A2 sgRNA 3
uccggcgacgcuuguccacu

SEQ ID NO: 30 - RNA, 20 nt, Artificial Sequence
  NR4A3 sgRNA 4
gcucgaguagcccuccacga

SEQ ID NO: 31 - DNA, 20 bp, Artificial Sequence
  gRNA 4
ggggtcccgtcggccgggtt

SEQ ID NO: 32 - DNA, 20 bp, Artificial Sequence
  gRNA 5
cccgtcggccgggttcggcg

SEQ ID NO: 33 - DNA, 20 bp, Artificial Sequence
  gRNA 6
ctttaggggtcccgtcggcc

SEQ ID NO: 34 - DNA, 20 bp, Artificial Sequence
  gRNA 7
cagaactttaggggtcccgt

SEQ ID NO: 35 - DNA, 20 bp, Artificial Sequence
  gRNA 8
actttaggggtcccgtcggc

SEQ ID NO: 36 - DNA, 20 bp, Artificial Sequence
  gRNA 9
agcgagcggggggctgcccc

SEQ ID NO: 37 - DNA, 20 bp, Artificial Sequence
  gRNA 10
cgcctccgccgccggagccc

SEQ ID NO: 38 - DNA, 20 bp, Artificial Sequence
  gRNA 11
cccgtcggccgggttcggcg

SEQ ID NO: 39 - DNA, 17 bp, Artificial Sequence
  S. aureus chimeric frame
taatacgactcactata

SEQ ID NO: 40 - DNA, 20 bp, Artificial Sequence
  gRNA 1
gtcaataccgccagaatcca

SEQ ID NO: 41 - DNA, 20 bp, Artificial Sequence
  gRNA 2
caataccgccagaatccatg

SEQ ID NO: 42 - DNA, 20 bp, Artificial Sequence
  gRNA 3
tcaataccgccagaatccat

SEQ ID NO: 43 - protein, 598 aa, Artificial Sequence
  NR4A2 Isoform 1
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 44 - protein, 535 aa, Artificial Sequence
  NR4A2 Isoform 2
MDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 45 - protein, 598 aa, Artificial Sequence
  NR4A1 Isoform 1
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 46 - protein, 611 aa, Artificial Sequence
  NR4A1 Isoform 2
MWLAKACWSIQSEMPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 47 - protein, 325 aa, Artificial Sequence
  NR4A1 Isoform 3
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKVPRSPRWGLLLEMERGWPHPIGTCGLPLGSPPS

SEQ ID NO: 48 - RNA, 20 nt, Artificial Sequence
  NR4A3 sgRNA 1
caauauagcccuuccccucc

SEQ ID NO: 49 - RNA, 20 nt, Artificial Sequence
  NR4A3 sgRNA 2
aacuggaaccuggaggggaa

SEQ ID NO: 50 - RNA, 20 nt, Artificial Sequence
  NR4A3 sgRNA 3
uaacuggaaccuggagggga

SEQ ID NO: 51 - skipped

SEQ ID NO: 52 - RNA, 20 nt, Artificial Sequence
  NR4A3 sgRNA 5
ccgcugcauuugguacacgc

SEQ ID NO: 53 - RNA, 20 nt, Artificial Sequence
  NR4A3 sgRNA 6
ugcggcgcagacauacagcu

SEQ ID NO: 54 - RNA, 20 nt, Artificial Sequence
  NR4A3 sgRNA 7
gcagcggcccuugaucaaag

SEQ ID NO: 55 - RNA, 20 nt, Artificial Sequence
  NR4A3 sgRNA 8
auacagcucggaauacacca

SEQ ID NO: 56 - RNA, 20 nt, Artificial Sequence
  NR4A3 sgRNA 9
ccugcguguaccaaaugcag

SEQ ID NO: 57 - RNA, 20 nt, Artificial Sequence
  NR4A3 sgRNA 10
gcggcccuugaucaaagugg

SEQ ID NO: 58 - RNA, 20 nt, Artificial Sequence
  NR4A3 sgRNA 11
ggacugcuugaaguacaugg

SEQ ID NO: 59 - RNA, 20 nt, Artificial Sequence
  NR4A3 sgRNA 12
cggguggcucucaagcgcgg

SEQ ID NO: 60 - RNA, 20 nt, Artificial Sequence
  NR4A3 sgRNA 13
gacgacgagcuccugcuggg

SEQ ID NO: 61 - RNA, 20 nt, Artificial Sequence
  NR4A3 sgRNA 14
gucgggguucaugaucuccg

SEQ ID NO: 62 - RNA, 20 nt, Artificial Sequence
  NR4A3 sgRNA 15
gagggcuugaaguggaagag

SEQ ID NO: 63 - RNA, 20 nt, Artificial Sequence
  NR4A3 sgRNA 16
gaugaaggcgguccccacgg

SEQ ID NO: 64 - RNA, 20 nt, Artificial Sequence
  NR4A3 sgRNA 17
gaagguacugaugcugggca

SEQ ID NO: 65 - RNA, 20 nt, Artificial Sequence
  NR4A3 sgRNA 18
uccuccagccuccagcccgg

SEQ ID NO: 66 - RNA, 20 nt, Artificial Sequence
  NR4A3 sgRNA 19
agcaucaguaccuucgugga

SEQ ID NO: 67 - RNA, 20 nt, Artificial Sequence
  NR4A3 sgRNA 20
cgacuacaccaagcugacca

SEQ ID NO: 68 - RNA, 20 nt, Artificial Sequence
  NR4A3 sgRNA 21
uggucagcuugguguagucg

SEQ ID NO: 69 - RNA, 20 nt, Artificial Sequence
  NR4A3 sgRNA 22
gcuggacccgccgaugaagg

SEQ ID NO: 70 - RNA, 20 nt, Artificial Sequence
  NR4A3 sgRNA 23
uugaaguacauggaggugcu

SEQ ID NO: 71 - RNA, 20 nt, Artificial Sequence
  NR4A3 sgRNA 24
guacggguggcucucaagcg

SEQ ID NO: 72 - RNA, 20 nt, Artificial Sequence
  NR4A3 sgRNA 25
ccgcauaacuggaaccugga

SEQ ID NO: 73 - RNA, 20 nt, Artificial Sequence
  NR4A3 sgRNA 26
gggcacgugugccgugugcg

SEQ ID NO: 74 - RNA, 20 nt, Artificial Sequence
  NR4A3 sgRNA 27
uacggcgugcgaaccugcga

SEQ ID NO: 75 - RNA, 20 nt, Artificial Sequence
  NR4A3 sgRNA 28
uggggacugcuugaaguaca

SEQ ID NO: 76 - RNA, 20 nt, Artificial Sequence
  NR4A3 sgRNA 29
ccuuggcagcacugagauca

SEQ ID NO: 77 - RNA, 20 nt, Artificial Sequence
  NR4A3 sgRNA 30
ccuugaucaaaguggaggag

SEQ ID NO: 78 - RNA, 20 nt, Artificial Sequence
  NR4A3 sgRNA 31
ugcauuugguacacgcagga

SEQ ID NO: 79 - RNA, 20 nt, Artificial Sequence
  NR4A3 sgRNA 32
ugaucaaaguggaggagggg

SEQ ID NO: 80 - RNA, 20 nt, Artificial Sequence
  NR4A3 sgRNA 33
guggggaccgccuucaucgg

SEQ ID NO: 81 - RNA, 20 nt, Artificial Sequence
  NR4A3 sgRNA 34
aggagcucgucgucuggcga

SEQ ID NO: 82 - RNA, 20 nt, Artificial Sequence
  NR4A3 sgRNA 35
ccaccucggcuacgacccga

SEQ ID NO: 83 - RNA, 20 nt, Artificial Sequence
  NR4A3 sgRNA 36
gcggcggcgagggcuugaag

SEQ ID NO: 84 - RNA, 20 nt, Artificial Sequence
  NR4A3 sgRNA 37
cagcaucaguaccuucgugg

SEQ ID NO: 85 - RNA, 20 nt, Artificial Sequence
  NR4A3 sgRNA 38
gccgaugaaggcggucccca

SEQ ID NO: 86 - RNA, 20 nt, Artificial Sequence
  NR4A3 sgRNA 39
ccgucgggucguagccgagg

SEQ ID NO: 87 - RNA, 20 nt, Artificial Sequence
  NR4A3 sgRNA 40
cuacggcgugcgaaccugcg

SEQ ID NO: 88 - RNA, 20 nt, Artificial Sequence
  NR4A3 sgRNA 41
ccauaacgcccccgccugcg

SEQ ID NO: 89 - RNA, 20 nt, Artificial Sequence
  NR4A3 sgRNA 42
auaacgcccccgccugcggg

SEQ ID NO: 90 - RNA, 20 nt, Artificial Sequence
  NR4A3 sgRNA 43
gccgcauaacuggaaccugg

SEQ ID NO: 91 - RNA, 20 nt, Artificial Sequence
  NR4A3 sgRNA 44
gaaaucgacaguacugacau

SEQ ID NO: 92 - RNA, 20 nt, Artificial Sequence
  NR4A3 sgRNA 45
uuucagaagugucucagugu

SEQ ID NO: 93 - RNA, 20 nt, Artificial Sequence
  NR4A3 sgRNA 46
gaagugucucaguguuggaa

SEQ ID NO: 94 - RNA, 20 nt, Artificial Sequence
  NR4A3 sgRNA 47
aguguuggaaugguaaaaga

SEQ ID NO: 95 - RNA, 20 nt, Artificial Sequence
  NR4A3 sgRNA 48
guacagauagucugaaaggg

SEQ ID NO: 96 - RNA, 20 nt, Artificial Sequence
  NR4A3 sgRNA 49
guguugagucuguuaaagcu

SEQ ID NO: 97 - RNA, 20 nt, Artificial Sequence
  NR4A3 sgRNA 50
gauagucugaaagggaggag

SEQ ID NO: 98 - RNA, 20 nt, Artificial Sequence
  NR4A3 sgRNA 51
agucuguuaaagcucggaca

SEQ ID NO: 99 - RNA, 20 nt, Artificial Sequence
  NR4A3 sgRNA 52
guccguacagauagucugaa

SEQ ID NO: 100 - DNA, 9 bp, Artificial Sequence
  NBRE
aaaaggtca

SEQ ID NO: 101-105 - skipped (5 entries)

SEQ ID NO: 106 - protein, 92 aa, Artificial Sequence
  NR4A1 DBD #1
SEGRCAVCGDNASCQHYGVRTCEGCKGFFKRTVQKNAKYICLANKDCPVDKRRRNRCQFCRFQKCLAVGMVKEVVRTDSLKGRRGRLPSKPK

SEQ ID NO: 107 - protein, 93 aa, Artificial Sequence
  NR4A1 DBD #2
SEGRCAVCGDNASCQHYGVRTCEGCKGFFKRTVQKNAKYICLANKDCPVDKRRRNRCQFCRFQKCLAVGMVKEVVRTDSLKGRRGRLPSKPKQ

SEQ ID NO: 108 - protein, 92 aa, Artificial Sequence
  NR4A2 DBD #1
NEGLCAVCGDNAACQHYGVRTCEGCKGFFKRTVQKNAKYVCLANKNCPVDKRRRNRCQYCRFQKCLAVGMVKEVVRTDSLKGRRGRLPSKPK

SEQ ID NO: 109 - protein, 93 aa, Artificial Sequence
  NR4A2 DBD #2
NEGLCAVCGDNAACQHYGVRTCEGCKGFFKRTVQKNAKYVCLANKNCPVDKRRRNRCQYCRFQKCLAVGMVKEVVRTDSLKGRRGRLPSKPKS

SEQ ID NO: 110 - protein, 92 aa, Artificial Sequence
  NR4A3 DBD #1
GEGTCAVCGDNAACQHYGVRTCEGCKGFFKRTVQKNAKYVCLANKNCPVDKRRRNRCQYCRFQKCLSVGMVKEVVRTDSLKGRRGRLPSKPK

SEQ ID NO: 111 - protein, 93 aa, Artificial Sequence
  NR4A3 DBD #2
GEGTCAVCGDNAACQHYGVRTCEGCKGFFKRTVQKNAKYVCLANKNCPVDKRRRNRCQYCRFQKCLSVGMVKEVVRTDSLKGRRGRLPSKPKS

SEQ ID NO: 112 - protein, 232 aa, Artificial Sequence
  DBD with NBRE-binding TAL
MLTPEQVVAIASNHGGKQALETVQRLLPVCQAHGLTPEQVVAIASNIGGKQALETVQRLLPVCQAHGLTPEQVVAIASNGGGKQALETVQRLLPVCQAHGLTPEQVVAIASNIGGKQALETVQRLLPVCQAHGLTPEQVVAIASNGGGKQALETVQRLLPVCQAHGLTPEQVVAIASNGGGKQALETVQRLLPVCQAHGLTPEQVVAIASNGGGKQALETVQRLLPVCQAHG

SEQ ID NO: 113 - protein, 232 aa, Artificial Sequence; identical to SEQ ID NO: 112
  DBD with NurRE-binding TAL #1

SEQ ID NO: 114 - protein, 231 aa, Artificial Sequence
  DBD with NurRE-binding TAL #2
LTPEQVVAIASNIGGKQALETVQRLLPVCQAHGLTPEQVVAIASNIGGKQALETVQRLLPVCQAHGLTPEQVVAIASNIGGKQALETVQRLLPVCQAHGLTPEQVVAIASNGGGKQALETVQRLLPVCQAHGLTPEQVVAIASNHGGKQALETVQRLLPVCQAHGLTPEQVVAIASHDGGKQALETVQRLLPVCQAHGLTPEQVVAIASHDGGKQALETVQRLLPVCQAHG

SEQ ID NO: 115 - DNA, 9 bp, Artificial Sequence
  ZFP binding sequence #1
aaaggtcaa

SEQ ID NO: 116 - skipped

SEQ ID NO: 117 - DNA, 6 bp, Artificial Sequence
  ZFP binding sequence #3
gatatt

SEQ ID NO: 118 - DNA, 6 bp, Artificial Sequence
  ZFP binding sequence #4
gccaat

SEQ ID NO: 119 - protein, 92 aa, Artificial Sequence
  DBD with ZFP #1
LEPGEKPYKCPECGKSFSQSGNLTEHQRTHTGEKPYKCPECGKSFSTSGHLVRHQRTHTGEKPYKCPECGKSFSQRANLRAHQRTHTGKKTS

SEQ ID NO: 120 - protein, 64 aa, Artificial Sequence
  DBD with ZFP #2
LEPGEKPYKCPECGKSFSHKNALQNHQRTHTGEKPYKCPECGKSFSTSGNLVRHQRTHTGKKTS

SEQ ID NO: 121 - protein, 64 aa, Artificial Sequence
  DBD with ZFP #3
LEPGEKPYKCPECGKSFSDCRDLARHQRTHTGEKPYKCPECGKSFSTTGNLTVHQRTHTGKKTS

SEQ ID NO: 122 - protein, 246 aa, Artificial Sequence
  NR4A LBD #1
PLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 123 - protein, 247 aa, Artificial Sequence
  NR4A LBD #2
SPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

(19) United States
(12) Patent Application Publication
(10) Pub. No.: US 2024/0374725 A1
(43) Pub. Date: Nov. 14, 2024

(54) NR4A3-DEFICIENT IMMUNE CELLS AND USES THEREOF

(71) Applicant: Lyell Immunopharma, Inc., South San Francisco, CA (US)

(72) Inventors: Viola LAM, South San Francisco, CA (US); Rachel Christina LYNN, South San Francisco, CA (US)

(73) Assignee: Lyell Immunopharma, Inc., South San Francisco, CA (US)

(21) Appl. No.: 18/564,163

(22) PCT Filed: May 27, 2022
(86) PCT No.: PCT/US2022/031356

§ 371 Date: Nov. 27, 2023

Related U.S. Application Data
(60) Provisional application No. 63/365,024, filed on May 19, 2022.
(60) Provisional application No. 63/194,745, filed on May 28, 2021.

(51) Int. Cl. A61K 39/00; A61P 35/00; C07K 14/705; C07K 14/725; C12N 9/22; C12N 15/11; C12N 15/90

(52) U.S. Cl. CPC A61K 39/4635; A61K 39/4611; A61K 39/4632; A61K 39/464488; A61P 35/00; C07K 14/7051; C07K 14/70567; C12N 9/22; C12N 15/11; C12N 15/907; C12N 2310/20; C12N 2510/00

(57) ABSTRACT

The preset disclosure provides methods of promoting a persistent effector function of immune cells, comprising modifying the cells to express reduced levels of NR4A3 gene and/or NR4A3 protein. Also provided are modified cells, e.g., immune cell, which have been modified to express reduced levels of NR4A3 gene and/or NR4A3 protein. Reducing levels of NR4A3 gene and/or NR4A3 protein leads to exhaustion/dysfunction resistant cells, which are apoptosis resistant and also immune checkpoint resistant, and also to the maintenance of anti-tumor function in tumor microenvironments.

### CROSS-REFERENCE TO RELATED APPLICATIONS

This PCT application claims the priority benefit of U.S. Provisional Application Nos. 63/194,745, filed May 28, 2021; and 63/365,024, filed May 19, 2022, each of which is herein incorporated by reference in its entirety.

### REFERENCE TO SEQUENCE LISTING SUBMITTED ELECTRONICALLY VIA EFS-WEB

The content of the electronically submitted sequence listing (Name: 4385_075PC02_Seqlisting_ST25.txt, Size: 97,201 bytes; and Date of Creation: May 27, 2022) submitted in this application is incorporated herein by reference in its entirety.

### FIELD OF THE DISCLOSURE

The present disclosure is related to cell-based (e.g., T-cell) cancer immunotherapy involving the administration of immune cells modified to have reduced levels of NR4A3 gene and/or reduced levels of NR4A3 protein expression.

### BACKGROUND OF THE DISCLOSURE

Cancer immunotherapy relies on getting T cells—the immune system's primary killers of infected and diseased cells—to attack and kill tumor cells. However, there is an important stumbling block for immunotherapy: T cells' ability to kill can fade, a phenomenon often referred to as exhaustion. Immune checkpoint blockade, chimeric antigen receptor (CAR) T cell therapy, and T cell receptor-engineered (TCR) T cell therapy are treatments that make use of functionally active T cells isolated from patients and require highly functional T cells in order to be effective. These T cells are engineered and expanded ex vivo to recognize specific antigens on target cancer cells.

When the immune system is forced to be active for extended periods, such as with persistent viral infections or the progressive development of cancer, effector T cells can become exhausted. One hallmark of exhausted T cells is the increased expression of immune checkpoint proteins like PD-1 and CTLA-4, which can cause those T cells to stand down (i.e., become non-functional). Immune checkpoint inhibitors block these checkpoint proteins and, in so doing, can increase the immune response against tumors. Some studies have suggested that blocking the activity of checkpoint proteins in exhausted T cells fails to achieve that end. This is important, because so-called hot tumors, those that include high levels of immune cells and thus should be ideal candidates to respond to immunotherapy, often harbor populations composed mostly of exhausted T cells. Moreover, tumor microenvironments can induce senescence and exhausted cellular phenotype. Therefore, devising strategies to reverse and/or prevent these exhausted states are crucial to improving immunotherapeutic efficacy.

### BRIEF SUMMARY OF THE DISCLOSURE

In some aspects, the present disclosure provides a method of treating a tumor in a subject in need thereof, comprising administering to the subject a cell composition comprising a population of modified immune cells that express reduced levels of Nuclear Receptor Subfamily 4 Group A Member 3 (“NR4A3”) gene and/or NR4A3 protein and a binding molecule (e.g., that specifically binds to ROR1) and that have endogenous expression of NR4A1 and NR4A2 genes and NR4A1 and NR4A2 proteins. In some aspects, the expression levels of NR4A3 gene and/or NR4A3 protein in the population of modified immune cells is reduced by at least about 5%, at least about 10%, at least about 20%, at least about 30%, at least about 40%, at least about 50%, at least about 60%, at least about 70%, at least about 80%, at least about 90%, or at least about 100%, compared to a reference cell composition (e.g., corresponding cell composition wherein the cells have not been modified to express lower levels of NR4A3 gene and/or NR4A3 protein). In one aspect, the expression level of the NR4A3 gene and/or protein is reduced by about 90% to about 99%. In one aspect the expression level of the NR4A3 gene and/or protein is reduced to below the level of detection.

In some aspects, the modified immune cells comprise lymphocytes, neutrophils, monocytes, macrophages, dendritic cells, and any combination thereof. In some aspects, the lymphocytes comprise T cells, tumor-infiltrating lymphocytes (TIL), lymphokine-activated killer cells, natural killer (NK) cells, and any combination thereof. In some aspects, the lymphocytes are T cells.

In some aspects, the binding molecule comprises a chimeric antigen receptor (CAR) and/or a T cell receptor (TCR), e.g., engineered TCR. In some aspects, the binding molecule comprises a CAR. In some aspects, the modified immune cells are ex vivo cells or in vitro cells. In some aspects, the modified immune cells are in vivo cells.

In some aspects, the modified immune cells are modified by a gene editing tool to reduce the expression of the NR4A3 gene and/or NR4A3 protein. In one aspect, the cells are modified to knock out the NR4A3 gene. In another aspect, the cells are modified to mutate the NR4A3 gene such that functional NR4A3 protein expression is abrogated. In some aspects, the gene editing tool comprises a shRNA, siRNA, miRNA, antisense oligonucleotides, CRISPR, zinc finger nuclease, TALEN, meganuclease, restriction endonuclease, or any combination thereof. In some aspects, the gene editing tool is CRISPR.

In some aspects, the population of modified immune cells exhibits one or more enhanced properties of the immune cells in the subject compared to the reference immune cells (i.e., immune cells that have not been modified to have reduced NR4A3 active gene levels and/or a reduced NR4A3 protein expression levels). In some aspects, the enhanced properties of the modified immune cell comprise (i) enhanced expansion of the immune cell, (ii) enhanced cytotoxicity of the immune cell, (iii) enhanced persistence, (iv) enhanced cytokine expression of the immune cell, or any combination thereof.

In some aspects, the modified immune cells exhibit enhanced cytokine expression. In some aspects, the cytokines are Interleukin-2 (IL-2), Interferon-γ (IFN-γ), Tumor necrosis factor-α (TNF-α), or any combination thereof. In some aspects, the expression level of IL-2 is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of IL-2 in a population of reference immune cells. In some aspects, the expression level of IFN-7 is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of IL-γ in a population of reference immune cells. In some aspects, the expression level of TNF-α is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of TNF-α in a population of reference immune cells.

In some aspects, the modified immune cells exhibit reduced exhaustion or dysfunction compared to the reference immune cells do (i.e., immune cells that have not been modified to have reduced NR4A3 active gene levels and/or a reduced NR4A3 protein expression levels). In some aspects, wherein the modified immune cells exhibit increased cytotoxicity upon sequential stimulation. In some aspects, the modified immune cells exhibit increased cytotoxicity in chronic stimulation. In some aspects, the modified immune cells maintain an anti-tumor function in tumor microenvironment (TME).

In some aspects, the binding molecule comprises an scFv derived from R12, R11, 2A2, or any combination thereof. In some aspects, the binding molecule comprises a heavy chain variable domain comprising SEQ ID NO: 17 and a light chain variable domain comprising SEQ ID NO: 21. In some aspects, the administering reduces a tumor volume in the subject, compared to a reference tumor volume (e.g., tumor volume in the subject prior to the administration and/or tumor volume in a subject that did not receive the administration). In some aspects, the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor volume (e.g., the tumor volume in the subject prior to the administration and/or tumor volume in a subject that did not receive the administration). In some aspects, the administering reduces a tumor weight in the subject, compared to a reference tumor weight (e.g., tumor weight in the subject prior to the administration and/or tumor weight in a subject that did not receive the administration). In some aspects, the tumor weight is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor weight (e.g., tumor weight in the subject prior to the administration and/or tumor weight in a subject that did not receive the administration).

In some aspects, the administering increases the duration of survival of the subject as compared to a reference duration of survival (e.g., duration of survival in the subject prior to the administration and/or duration of survival in a subject that did not receive the administration). In some aspects, compared to the reference duration of survival, the duration of survival is increased by at least about one week, at least about two weeks, at least about three weeks, at least about one month, at least about two months, at least about three months, at least about four months, at least about five months, at least about six months, at least about seven months, at least about eight months, at least about nine months, at least about 10 months, at least about 11 months, or at least about one year.

In some aspects, the administering reduces or prevents exhaustion or dysfunction of the immune cells. In some aspects, the immune cells maintain an anti-tumor function in tumor microenvironment (TME). In some aspects, the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, cervical cancer, carcinoma, sarcoma, leukemia, lymphoma, myeloma, or a combination thereof.

In some aspects, the method further comprises administering an additional therapeutic agent to the subject. In some aspects, the additional therapeutic agent comprises a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the additional therapeutic agent is an immune checkpoint inhibitor. In some aspects, the immune checkpoint inhibitor comprises an anti-PD-1 antibody, an anti-PD-L1 antibody, an anti-LAG-3 antibody, an anti-CTLA-4 antibody, an anti-GITR antibody, an anti-TIM3 antibody, and any combination thereof. In some aspects, wherein the additional therapeutic agent and the cell composition are administered concurrently. In some aspects, the additional therapeutic agent and the cell composition are administered sequentially.

In some aspects, the cell composition are administered parenterally, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

For any of the above methods, in some aspects, the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

In some aspects, the present disclosure provides a method of generating a cell having a reduced level of Nuclear Receptor Subfamily 4 Group A Member 3 “(NR4A3”) gene and/or NR4A3 protein, comprising modifying the cells with a gene editing tool, wherein the gene editing tool reduces the expression of the NR4A3 gene and/or NR4A3 protein, wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

In some aspects, the present disclosure also provides a method of increasing the production of a cytokine by immune cells in response to an antigen stimulation, comprising modifying the immune cells with a gene editing tool, wherein the gene editing tool reduces the expression of the NR4A3 gene and/or NR4A3 protein, and wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96. In some aspects, the cytokine comprises IFN-γ, IL-2, TNF-α, or a combination thereof. In some aspects, after the modification, the production of the cytokine in response to the antigen stimulation is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, or at least about 50-fold, compared to corresponding immune cells that were not modified with the gene editing tool.

In some aspects, the present application provides a method of increasing an effector function of immune cells in response to persistent antigen stimulation, comprising modifying the immune cells with a gene editing tool, wherein the gene editing tool reduces the expression of the NR4A3 gene and/or NR4A3 protein, and wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96. In some aspects, the immune cells retain effector function for at least one, at least two, or at least three additional rounds of an antigen stimulation assay, as compared to reference immune cells. In some aspects, the effector function comprises the ability: (i) to kill target cells (e.g., tumor cells) (ii) to produce a cytokine upon further antigen stimulation, or (iii) both (i) and (ii).

In some aspects, the guide RNA comprises, consists of, or consists essentially of the sequence set forth in SEQ ID NO: 30. In some aspects, the guide RNA comprises, consists of, or consists essentially of the sequence set forth in SEQ ID NO: 52. In some aspects, the guide RNA comprises, consists of, or consists essentially of the sequence set forth in SEQ ID NO: 53. In some aspects, the guide RNA comprises, consists of, or consists essentially of the sequence set forth in SEQ ID NO: 54. In some aspects, the guide RNA comprises, consists of, or consists essentially of the sequence set forth in SEQ ID NO: 55. In some aspects, the guide RNA comprises, consists of, or consists essentially of the sequence set forth in SEQ ID NO: 56. In some aspects, the guide RNA comprises, consists of, or consists essentially of the sequence set forth in SEQ ID NO: 57. In some aspects, the guide RNA comprises, consists of, or consists essentially of the sequence set forth in SEQ ID NO: 58. In some aspects, the guide RNA comprises, consists of, or consists essentially of the sequence set forth in SEQ ID NO: 61. In some aspects, the guide RNA comprises, consists of, or consists essentially of the sequence set forth in SEQ ID NO: 65. In some aspects, the guide RNA comprises, consists of, or consists essentially of the sequence set forth in SEQ ID NO: 67. In some aspects, the guide RNA comprises, consists of, or consists essentially of the sequence set forth in SEQ ID NO: 68. In some aspects, the guide RNA comprises, consists of, or consists essentially of the sequence set forth in SEQ ID NO: 70. In some aspects, the guide RNA comprises, consists of, or consists essentially of the sequence set forth in SEQ ID NO: 71. In some aspects, the guide RNA comprises, consists of, or consists essentially of the sequence set forth in SEQ ID NO: 75. In some aspects, the guide RNA comprises, consists of, or consists essentially of the sequence set forth in SEQ ID NO: 76. In some aspects, the guide RNA comprises, consists of, or consists essentially of the sequence set forth in SEQ ID NO: 82. In some aspects, the guide RNA comprises, consists of, or consists essentially of the sequence set forth in SEQ ID NO: 83. In some aspects, the guide RNA comprises, consists of, or consists essentially of the sequence set forth in SEQ ID NO: 86. In some aspects, the guide RNA comprises, consists of, or consists essentially of the sequence set forth in SEQ ID NO: 94. In some aspects, the guide RNA comprises, consists of, or consists essentially of the sequence set forth in SEQ ID NO: 96.

In some aspects, the present disclosure provides a composition comprising a cell, wherein the cell has been prepared by the method of claim 43. In some aspects, provided herein is a composition comprising a cell which expresses a reduced level of a NR4A3 gene and/or NR4A3 protein, wherein the cell has been modified with a gRNA that can target the NR4A3 gene, wherein the gRNA comprises, consists of, or consists essentially of the sequence set forth in any one of SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

In some aspects, the guide RNA comprises, consists of, or consists essentially of the sequence set forth in SEQ ID NO: 30. In some aspects, the guide RNA comprises, consists of, or consists essentially of the sequence set forth in SEQ ID NO: 52. In some aspects, the guide RNA comprises, consists of, or consists essentially of the sequence set forth in SEQ ID NO: 53. In some aspects, the guide RNA comprises, consists of, or consists essentially of the sequence set forth in SEQ ID NO: 54. In some aspects, the guide RNA comprises, consists of, or consists essentially of the sequence set forth in SEQ ID NO: 55. In some aspects, the guide RNA comprises, consists of, or consists essentially of the sequence set forth in SEQ ID NO: 56. In some aspects, the guide RNA comprises, consists of, or consists essentially of the sequence set forth in SEQ ID NO: 57. In some aspects, the guide RNA comprises, consists of, or consists essentially of the sequence set forth in SEQ ID NO: 58. In some aspects, the guide RNA comprises, consists of, or consists essentially of the sequence set forth in SEQ ID NO: 61. In some aspects, the guide RNA comprises, consists of, or consists essentially of the sequence set forth in SEQ ID NO: 65. In some aspects, the guide RNA comprises, consists of, or consists essentially of the sequence set forth in SEQ ID NO: 67. In some aspects, the guide RNA comprises, consists of, or consists essentially of the sequence set forth in SEQ ID NO: 68. In some aspects, the guide RNA comprises, consists of, or consists essentially of the sequence set forth in SEQ ID NO: 70. In some aspects, the guide RNA comprises, consists of, or consists essentially of the sequence set forth in SEQ ID NO: 71. In some aspects, the guide RNA comprises, consists of, or consists essentially of the sequence set forth in SEQ ID NO: 75. In some aspects, the guide RNA comprises, consists of, or consists essentially of the sequence set forth in SEQ ID NO: 76. In some aspects, the guide RNA comprises, consists of, or consists essentially of the sequence set forth in SEQ ID NO: 82. In some aspects, the guide RNA comprises, consists of, or consists essentially of the sequence set forth in SEQ ID NO: 83. In some aspects, the guide RNA comprises, consists of, or consists essentially of the sequence set forth in SEQ ID NO: 86. In some aspects, the guide RNA comprises, consists of, or consists essentially of the sequence set forth in SEQ ID NO: 94. In some aspects, the guide RNA comprises, consists of, or consists essentially of the sequence set forth in SEQ ID NO: 96.

In some aspects, the present disclosure provides a cell prepared by the methods described herein. In some aspects, the cell is an in vivo cell. In some aspects, the cell is an ex vivo or an in vitro cell.

In some aspects, the present disclosure provides pharmaceutical compositions comprising the cells described herein.

In some aspects, the present disclosure provides a kit comprising (i) a gene editing tool to reduce the expression of NR4A3 gene and/or NR4A3 protein, (ii) a vector comprising a chimeric antigen receptor (CAR) or a T cell receptor (TCR), and instructions for treating tumor according to the methods described herein.

In some aspects, the present disclosure provides a kit comprising (i) a gene editing tool to reduce the expression of NR4A3 gene and/or NR4A3 protein, (ii) a vector comprising a chimeric antigen receptor (CAR) or a T cell receptor (TCR), and instructions for preparing a cell composition according to the methods described herein.

### BRIEF DESCRIPTION OF THE DRAWINGS/FIGURES

FIGS. 1A and 1B show the percentage of NR4A3 expression in NR4A3-edited (“NR4A3 KO”) and control non-edited CD4+(FIG. 1A) and CD8+(FIG. 1B) anti-ROR1 CAR T cells on day 7 of CAR T cell production following a 2-hour CD3/CD28 Dynabead stimulation in five independent donors (Stim, black circles). Unstim cells (white circles, without Dynabeads) were used as a negative control. Unpaired t-test of stimulated conditions were used for statistical analysis. *p<0.05, ** p<0.005, *** p<0.001, **** p<0.0001.

FIG. 2 shows the percentage of EGFR⁺R12⁺ ROR1 CAR expression in NR4A1-(“NR4A1 KO”), NR4A2-(“NR4A2 KO”), NR4A3-edited (“NR4A3 KO”), and control non-edited CD4⁺ (white circles) and CD8+(black circles) anti-ROR1 CAR T cells from five donors on day 7 of CAR T cell production.

FIG. 3 shows successive anti-ROR1 lysis of H1975-NLR NSCLC cells by NR4A-edited or control non-edited anti-ROR1 CAR T cells (“x” symbol), and mock untransduced T cells (square) in five independent donors in the sequential stimulation assays. The NR4A-edited cells shown include: NR4A1 knockout (triangle), NR4A2 knockout (star), and NR4A3 knockout (circle). Lysis of H1975-NLR target cells were quantified by measuring total NLR intensity. NLR intensity was normalized relative to the starting intensity after replating for each round of stimulation. NLR—NucLight Red.

FIGS. 4A-4C show secreted interferon-gamma (IFN-γ) (FIG. 4A), interleukin-2 (IL-2) (FIG. 4B), and tumor-necrosis factor-alpha (TNF-α) (FIG. 4C) produced from NR4A-edited, control non-edited anti-ROR1 CAR (“x” symbol), and mock untransduced T cells (square) during the H1975 sequential stimulation assay corresponding to FIG. 3. The NR4A-edited cells shown include: NR4A1 knockout (triangle), NR4A2 knockout (star), and NR4A3 knockout (circle). Supernatants were collected 24 hours after each replating and cytokines were quantified by MSD. Columns show data from 5 independent donors. Error bars represent mean+/−SD of triplicate wells. Unpaired t-test was used for statistical analysis. *p<0.05, **p<0.005, *** p<0.001, **** p<0.0001.

FIG. 5A shows anti-ROR1 CAR expression on CD4⁺ (upper) and CD8⁺ (lower) T cells from NR4A-edited, control non-edited ROR1 CAR (“x” symbol), and mock untransduced (square) T cells after each replating during the H1975 sequential stimulation assay corresponding to FIG. 3. The NR4A-edited cells shown include: NR4A1 knockout (triangle), NR4A2 knockout (star), and NR4A3 knockout (circle). D0 shows the frequency of CAR expression at the beginning of the assay. FIG. 5B shows fold change in projected CD3⁺ anti-ROR1 CAR⁺ T cell numbers from NR4A-edited and control non-edited anti-ROR1 CAR T cells during the H1975 sequential stimulation assay corresponding to FIG. 3. Projected cell numbers were calculated to include the 25% transfer of the cells to the next stimulation. Fold change was calculated as (projected cell numbers from stimulation/projected cell numbers from previous stimulation). In FIG. 5B, the bars in each of Stim1, Stim2, and Stim3 correspond to the following: (i) NR4A1 knockout (triangle; first bar); (ii) NR4A2 knockout (star; second bar); (iii) NR4A3 knockout (circle; third bar); and (iv) control non-edited ROR1 CAR (x symbol; fourth bar). Each of the graphs represents an independent donor.

FIG. 6 shows the expression of inhibitory receptors (LAG3, TIM3, CD39, and PD-1) on ROR1 CAR⁺ CD4⁺ (upper) and CD8⁺ (lower) T cells from NR4A3-edited (“NR4A3 KO”) and control non-edited anti-ROR1 CAR cells from the H1975 sequential stimulation assay corresponding to the second stimulation in FIG. 3. Paired t-test was used for statistical analysis. ** p<0.005. n=5 independent donors.

FIGS. 7A-7C show secreted interferon-gamma (IFN-γ) (FIG. 7A), interleukin-2 (IL-2) (FIG. 7B), and tumor-necrosis factor-alpha (TNF-α) (FIG. 7C) produced from NR4A-edited and control non-edited anti-ROR1 CAR T cells co-cultured with A549 (top row) or H1975 (bottom row) tumor cells in five independent donors following seven days of ROR1 antigen stimulation in the H1975 chronic stimulation assay. Supernatants were collected 24 hours after fresh co-culture set up on day seven and cytokines were quantified by MSD. Each of the graphs represents an independent donor. In each of the graphs, the bars correspond to the following: (i) NR4A1 knockout (triangle; first bar); (ii) NR4A2 knockout (star; second bar); (iii) NR4A3 knockout (circle; third bar); and (iv) control non-edited anti-ROR1 CAR (x symbol; fourth bar). Columns show data from 5 independent donors. Error bars represent mean+/−SD of triplicate wells. Unpaired t-test was used for statistical analysis. *p<0.05, ** p<0.005, *** p<0.001, **** p<0.0001.

FIG. 8A shows ROR1 CAR expression on CD4⁺ (upper) and CD8⁺ (lower) T cells from NR4A-edited and control non-edited ROR1 CAR T cells during the H1975 chronic stimulation assay corresponding to FIG. 7. The NR4A-edited cells shown include: NR4A1 knockout (triangle), NR4A2 knockout (star), and NR4A3 knockout (circle). FIG. 8B shows fold change in CD3⁺ anti-ROR1 CAR⁺ T cell numbers from NR4A-edited and control non-edited ROR1 CAR T cells during the seven-day H1975 chronic stimulation assay corresponding to FIG. 7. Fold change was calculated as (cell numbers from current E:T reset/previous E:T reset cell numbers). Each of the graphs represents an independent donor. In FIG. 8B, the bars shown for each of Day 2, Day 4, and Day 7, correspond to the following: (i) NR4A1 knockout (triangle; first bar); (ii) NR4A2 knockout (star; second bar); (iii) NR4A3 knockout (circle; third bar); and (iv) control non-edited ROR1 CAR (x symbol; fourth bar). Columns show data from 5 independent donors.

FIG. 9 shows the expression of inhibitory receptors (LAG3, TIM3, CD39, and PD-1) on anti-ROR1 CAR⁺ CD4⁺ (upper) and CD8⁺ (lower) T cells from NR4A3-edited (“NR4A3 KO”) and control non-edited anti-ROR1 CAR cells on day seven of the H1975 chronic stimulation assay corresponding to FIG. 7. n=5 independent donors.

FIGS. 10A and 10B show improved in vivo efficacy of NR4A3-edited anti-ROR1 CAR T cells. FIG. 10A shows tumor volume and FIG. 10B shows survival of NSG mice implanted with subcutaneous flank H1975 xenograft tumors. Mice were treated i.v. with NR4A-edited or control non-edited anti-ROR1 CAR T cells at either 0.6×10⁶ (upper panels, low dose) or 2×10⁶ (lower panels, high dose) CAR⁺ T cells per mouse when mean tumor volumes reached 80-120 mm³. n=5 mice per group. Error bars represent mean+/−SEM. Survival curve statistics calculated by log-rank (Manel-Cox) test. * ** p<0.001 and *** p<0.0001. The different groups shown include: (i) NR4A1 knockout (triangle), (ii) NR4A2 knockout (star), (iii) NR4A3 knockout (circle), (iv) control non-edited anti-ROR1 CAR (x symbol), and mock non-transduced T cells (square).

FIGS. 11A and 11B shows successive lysis of A549-NLR and H1975-NLR cells, respectively, by anti-ROR1 CAR T cells that were modified to have reduced levels of the following multiple members of the NR4A family: (1) both NR4A1 and NR4A2 (“NR4A 1+2 DKO”) (open circle), (2) both NR4A1 and NR4A3 (“NR4A 1+3 DKO”) (triangle), (3) both NR4A2 and NR4A3 (“NR4A 2+3 DKO”) (star), and (4) NR4A1, NR4A2, and NR4A3 (“NR4A TKO”) (asterisk). Anti-ROR1 CAR T cells with reduced level of only NR4A3 (closed circle) are also shown for comparison purposes. Mock (untransduced T cells without ROR1 CAR or NR4A editing) (square) is shown as a control. Lysis of H1975-NLR target cells were quantified by measuring total NLR intensity. NLR intensity was normalized relative to the starting intensity after replating for each round of stimulation. NLR—NucLight Red.

FIGS. 12A-12C show IFN-γ (FIG. 12A), IL-2 (FIG. 12B), and TNF-α (FIG. 12C) levels produced by anti-ROR1 CAR T cells with reduced level of multiple members of the NR4A family during a sequential stimulation assay (see FIG. 11A) using A549 target cells. The NR4A-edited anti-ROR1 CAR T cells and control groups are the same as that described in FIGS. 11A and 11B. Supernatants were collected 24 hours after each replating (i.e., stim 1, stim 2, stim 3, stim 4, and stim 5) and cytokines were quantified by MSD.

FIGS. 13A-13C show IFN-γ (FIG. 13A), IL-2 (FIG. 13B), and TNF-α (FIG. 13C) levels produced by anti-ROR1 CAR T cells with reduced level of multiple members of the NR4A family during a sequential stimulation assay (see FIG. 11B) using H1975 target cells. The NR4A-edited anti-ROR1 CAR T cells and control groups are the same as that described in FIGS. 11A and 11B. Supernatants were collected 24 hours after each replating (i.e., stim 1, stim 2, stim 3, stim 4, and stim 5) and cytokines were quantified by MSD.

FIG. 14 shows successive lysis of NY-ESO-1+ A375-NLR melanoma cells by NR4A-edited (KO), control non-edited NY-ESO-1 TCR T cells and mock untransduced T cells in three independent donors in the sequential stimulation assay. Lysis of A375-NLR target cells were quantified by measuring total NLR count. NLR count was normalized relative to the starting count after replating for each round of stimulation. NLR—NucLight Red. Each graph shows data from three independent donors.

FIGS. 15A-15C show secreted interferon-gamma (IFN-γ) (FIG. 15A), interleukin-2 (IL-2) (FIG. 15B), and tumor-necrosis factor-alpha (TNF-α) (FIG. 15C) produced from NR4A-edited, control non-edited NY-ESO-1 TCR T cells, and mock untransduced T cells during the A375 sequential stimulation assay corresponding to FIG. 14. Supernatants were collected 24 hours after each replating and cytokines were quantified by MSD. Graphs show data from 3 independent donors. In each of FIGS. 15A-15C, for each of the stimulations (i.e., Stim 1, Stim 2, Stim 3, and Stim 4), the first bar (from the left) is the NR4A1-edited NY-ESO-1 TCR T cells (NR4A1 KO; triangle), the second bar is the NR4A2-edited NY-ESO-1 TCR T cells (NR4A2 KO; asterisk); the third bar is the NR4A3-edited NY-ESO-1 TCR T cells (NR4A3 KO; closed circle); the fourth bar is the non-edited control NY-ESO-1 TCR T cells (Control; x symbol); and the fifth bar is the untransduced T cells (mock; square).

### DETAILED DESCRIPTION OF THE DISCLOSURE

The present disclosure is directed to methods of treating a disease in a subject in need thereof comprising administering to the subject a composition comprising a population of modified immune cells that express reduced levels of Nuclear Receptor Subfamily 4 Group A Member 3 (NR4A3) gene and/or NR4A3 protein and that have endogenous expression of NR4A1 and NR4A2 genes and NR4A1 and NR4A2 proteins. The reduction in levels of NR4A3 gene can be accomplished by using gene editing techniques, e.g., gene editing techniques such as CRISPR. As will be apparent from the present disclosure and demonstrated herein, in some aspects, the modified immune cells with reduced level of NR4A3 gene and/or NR4A3 protein can also express reduced level of other members of the NR4A family. Accordingly, in some aspects, modified immune cells useful for the present disclosure has: (i) reduced level of NR4A3 gene and/or NR4A3 protein and (ii) reduced level of NR4A1 gene and/or NR4A1 protein. In some aspects, modified immune cells useful for the present disclosure has: (i) reduced level of NR4A3 gene and/or NR4A3 protein and (ii) reduced level of NR4A2 gene and/or NR4A2 protein. In some aspects, modified immune cells useful for the present disclosure has: (i) reduced level of NR4A3 gene and/or NR4A3 protein, (ii) reduced level of NR4A1 gene and/or NR4A1 protein, and (iii) reduced level of NR4A2 gene and/or NR4A2 protein.

Reduction in levels of NR4A3 gene and/or NR4A3 protein expression (alone or in combination with reduced level of the NR4A1 gene and/or NR4A1 protein and/or the NR4A2 gene and/or NR4A2 protein) can lead to one or more persistent effector functions. One aspect of the persistent effector functions is enhanced T cell activation (e.g., enhanced expansion, enhanced cytotoxicity, enhanced cytokine expression). Reducing levels of NR4A3 gene and/or NR4A3 protein (alone or in combination with reduced level of the NR4A1 gene and/or NR4A1 protein and/or the NR4A2 gene and/or NR4A2 protein) can lead to exhaustion/dysfunction resistant cells. Furthermore, reducing levels of NR4A3 gene and/or NR4A3 protein (alone or in combination with reduced level of the NR4A1 gene and/or NR4A1 protein and/or the NR4A2 gene and/or NR4A2 protein) can result in the maintenance of anti-tumor function in TME environments.

The disclosure also provides, e.g., methods to generate modified immune cells that express reduced levels of Nuclear Receptor Subfamily 4 Group A Member 3 (NR4A3) gene and/or NR4A3 protein and endogenous levels of NR4A1 and NR4A2 gene and NR4A1 and NR4A2 protein, methods to use the modified immune cells, pharmaceutical compositions, or kits. As described herein, in some aspects, the modified immune cells useful for such methods, pharmaceutical compositions, or kits have been further modified to have: (i) reduced level of the NR4A1 gene and/or NR4A1 protein, (ii) reduced level of NR4A2 gene and/or NR4A2 protein, or (iii) both (i) and (ii).

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an immune cell,” is understood to represent one or more immune cells. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In some aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell).

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

It is to be understood that in the disclosed sequences T and U are interchangeable depending on whether the sequence is a DNA or an RNA. For example, gRNA spacer sequences are presented as DNAs (A/T/C/G) in the present disclosure, whereas the gRNA chimeric frames are presented as RNAs (A/U/C/G).

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

The term “polynucleotide” as used herein refer to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including mRNAs and gRNAs, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. Unless indicated otherwise, the term “polynucleotides,” “nucleic acids,” “gene,” “cDNA,” and “mRNA” can be used interchangeably.

The term “gene” means the segment of DNA involved in producing a polypeptide chain. It can include regions preceding and following the coding region (leader and trailer) as well as intervening sequences (introns) between individual coding segments (exons).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a T_{reg} cell. As used herein, the term “T cell” and “T lymphocytes” are interchangeable and refer to any lymphocytes produced or processed by the thymus gland. In some aspects, a T cell is a CD4⁺ T cell. In some aspects, a T cell is a CD8⁺ T cell. In some aspects, a T cell is a NKT cell.

As used herein, the term “anti-tumor immune response” refers to an immune response against a tumor antigen. An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. In some aspects, the ability to stimulate an immune response or the immune system activity can be enhanced, e.g., by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some aspects, the ability to stimulate an immune response or the immune system activity can be enhanced, e.g., at least about 1.2 fold, at least about 1.4 fold, at least about 1.6 fold, at least about 1.8 fold, at least about 2 fold, at least about 3 fold, at least about 4 fold, at least about 5 fold, at least about 6 fold, at least about 7 fold, at least about 8 fold, at least about 9 fold, at least about 10 fold, or more.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., a modified immune cells disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the modified cell herein clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In some aspects, a therapeutically effective amount of the drug promotes cancer regression to the point of eliminating the cancer.

“Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

As used herein, the term “oxidative stress” refers to the condition characterized by an excess of oxidants and/or a decrease in antioxidant levels. Cellular oxidants can include, but are not limited to, radicals of oxygen (superoxide anion, hydroxyl radical, and/or peroxy radicals); reactive non-radical oxygen species such as, for example, hydrogen peroxide and singlet oxygen; carbon radicals; nitrogen radicals; sulfur radicals; and combinations thereof. In some aspects, the condition of oxidative stress can result in, for example, cellular damage, impaired performance of cells, and/or cell death.

As used herein, the term “modified cell” refers to a cell, e.g., a T cell, that has undergone non naturally-occurring engineering so that a phenotype of the cell (i.e., expression level of a NR4A3 gene and/or NR4A3 protein) is different from the unmodified cell (i.e., reference cell). As will be apparent from the disclosure, modified cells disclosed herein express reduced levels of NR4A3 gene and/or NR4A3 protein compared to reference cells (e.g., corresponding cells that have not been modified). As described herein, in some aspects, the modified cells can express normal levels of the NR4A1 gene and/or NR4A1 protein and the NR4A2 gene and/or NR4A2 protein. In some aspects, the modified cells of the present disclosure can express: (i) reduced level of NR4A3 gene and/or NR4A3 protein and (ii) reduced level of NR4A1 gene and/or NR4A1 protein. In some aspects, the modified cells described herein can express: (i) reduced level of NR4A3 gene and/or NR4A3 protein and (ii) reduced level of NR4A2 gene and/or NR4A2 protein. In some aspects, the modified cells can express: (i) reduced level of NR4A3 gene and/or NR4A3 protein, (ii) reduced level of NR4A1 gene and/or NR4A1 protein, and (iii) reduced level of NR4A2 gene and/or NR4A2 protein. As used herein, the term “corresponding cell” refers to a cell that belongs to the same immune cell classification as the modified cell. For example, if the modified cell is a T cell, the corresponding cell would also be a T cell. Unless indicated otherwise, “modified cells having (expressing) reduced level of NR4A3 gene and/or NR4A3 protein” (including variants thereof) comprise cells (e.g., T cells) that have been modified to have reduced level of NR4A3 gene and/or NR4A3 protein and: (i) endogenous level of NR4A1 and NR4A2 genes and NR4A1 and NR4A2 proteins; (ii) reduced level of NR4A1 gene and/or NR4A1 protein; (iii) reduced level of NR4A2 gene and/or NR4A2 protein; or (iv) reduced level of both the NR4A1 gene and/or NR4A1 protein and the NR4A2 gene and/or NR4A2 protein.

As used herein, the term “endogenous expression” or “endogenous expression levels” or “endogenous levels” (or variants thereof) refers to gene and/or protein expression (e.g., amount, kinetics, etc.) that is naturally occurring (e.g., the gene and/or protein is not directly manipulated by non-naturally-occurring engineering). As will be apparent from the disclosure, in some aspects, a modified cell disclosed herein (e.g., ROR1 CAR T cell with NR4A3 knocked down) does not express endogenous levels of NR4A3 gene or NR4A3 protein, but because the NR4A1 and NR4A2 genes have not been knocked down (e.g., by CRISPR, e.g., a non-naturally occurring engineering) the modified cells endogenously express NR4A1 and NR4A2 gene and/or NR4A1 and NR4A2 protein. As described herein, in some aspects, the modified cells expressing reduced level of the NR4A3 gene or NR4A3 protein can be further modified to also express reduced level of: (i) the NR4A1 gene or NR4A1 protein, (ii) the NR4A2 gene or NR4A2 protein, or (iii) both (i) and (ii).

In some aspects, a modified cell is produced by introducing a foreign or exogenous nucleic acid into a cell. In some aspects, the foreign or exogenous nucleic acid can encode a gene editing tool disclosed herein. A nucleic acid can be introduced into a cell by methods known in the art, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), or microinjection of purified DNA directly into the nucleus of the cell (see, e.g., Capecchi M. R. Cell. 1980 November; 22(2 Pt 2): 479-88).

It is to be understood that disclosures referring to a “modified cell” or to a “cell” are equally applicable to a population of those cells, i.e., to a plurality of those cells.

As used herein, the terms “elevated concentrations” or “elevated levels” and grammatical variants thereof refer to above-normal levels of a substance (e.g., a reactive oxygen species; ROS) compared to appropriate controls (e.g., healthy tissue or cells).

As used herein, the terms “reactive oxygen species” and “ROS” refer to highly reactive chemicals, containing oxygen, that react easily with other molecules, resulting in potentially damaging modifications. Reactive oxygen species include, for example, oxygen ions, free radicals and peroxides both inorganic and organic such as hydrogen peroxide, superoxide, hydroxyl radical, lipid hydroperoxidase and singlet oxygen. They are generally very small molecules and are highly reactive due to the presence of unpaired valence shell electrons. Nearly all cancers are associated with elevated concentrations of reactive oxygen species. Liou, G., et al., Free Radic Res 44(5): 1-31 (2010).

The terms “chimeric antigen receptor” and “CAR,” as used herein, refer to a recombinant fusion protein that has an antigen-specific extracellular domain coupled to an intracellular domain that directs the cell to perform a specialized function upon binding of an antigen to the extracellular domain. The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MHC-independent antigen and transduce activation signals via their intracellular domain.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 pM to about 10 μM, for example, about 0.1 pM to about 1 pM or about 0.1 μM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some aspects, T cell receptor (TCR) based recognition domains, such as single chain TCR (scTv, single chain two-domain TCR containing V.alpha.V.beta.) are also suitable for use.

A chimeric antigen receptor disclosed herein can also include an intracellular domain that provides an intracellular signal to the cell (expressing the CAR) upon antigen binding to the antigen-specific extracellular domain. In some aspects, the intracellular signaling domain of a CAR is responsible for activation of at least one of the effector functions of the T cell in which the chimeric receptor is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, Fc RIII, Fc RI, and combinations of signaling molecules, such as CD3.zeta. and CD28, CD27, 4-1BB, DAP-10, OX40, and combinations thereof, as well as other similar molecules and fragments. Intracellular signaling portions of other members of the families of activating proteins can be used, such as FcγRIII and FcεRI. While usually the entire intracellular domain will be employed, in many cases it will not be necessary to use the entire intracellular polypeptide. To the extent that a truncated portion of the intracellular signaling domain can find use, such truncated portion can be used in place of the intact chain as long as it still transduces the effector function signal. The term intracellular domain is thus meant to include any truncated portion of the intracellular domain sufficient to transduce the effector function signal. Typically, the antigen-specific extracellular domain is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. Chimeric antigen receptors can also further comprise one or more costimulatory domain and/or one or more spacer. A costimulatory domain is derived from the intracellular signaling domains of costimulatory proteins that enhance cytokine production, proliferation, cytotoxicity, and/or persistence in vivo.

A “peptide hinge” or “spacer” connects the antigen-specific extracellular domain to the transmembrane domain. The transmembrane domain is fused to the costimulatory domain, optionally a costimulatory domain is fused to a second costimulatory domain, and the costimulatory domain is fused to a signaling domain, not limited to CD3ζ For example, inclusion of a spacer domain between the antigen-specific extracellular domain and the transmembrane domain, and between multiple scFvs in the case of tandem CAR, can affect flexibility of the antigen-binding domain(s) and thereby CAR function. Suitable transmembrane domains, costimulatory domains, and spacers are known in the art.

As used herein, the terms “ug” and “uM” are used interchangeably with”μg” and “μM,” respectively.

As used herein, the term “gene-editing” refers to the process of changing the genetic information present in the genome of a cell. This gene-editing can be performed by manipulating genomic DNA, resulting in a modification of the genetic information. In some aspects, such gene-editing can influence expression of the DNA that has been edited. In some aspects, such gene-editing does not affect the expression of the DNA that has been edited. In some aspects, gene-editing of a modified cell disclosed herein can be done using a gene editing tool described herein. Non-limiting examples of gene editing tools include RNA interference molecules (e.g., shRNA, siRNA, miRNA), antisense oligonucleotides, CRISPR, zinc finger nuclease (ZFN), transcription activator-like effector nuclease (TALEN), meganucleases, restriction endonuclease, or any combination thereof.

As used herein, the term “nuclease” refers to an enzyme which possesses catalytic activity for DNA cleavage. Any nuclease agent that induces a nick or double-strand break into a desired recognition site can be used in the methods and compositions disclosed herein. A naturally-occurring or native nuclease agent can be employed so long as the nuclease agent induces a nick or double-strand break in a desired recognition site. Alternatively, a modified or engineered nuclease agent can be employed. An “engineered nuclease agent” comprises a nuclease that is engineered (modified or derived) from its native form to specifically recognize and induce a nick or double-strand break in the desired recognition site. Thus, an engineered nuclease agent can be derived from a native, naturally-occurring nuclease agent or it can be artificially created or synthesized. The modification of the nuclease agent can be as little as one amino acid in a protein cleavage agent or one nucleotide in a nucleic acid cleavage agent. In some aspects, the engineered nuclease induces a nick or double-strand break in a recognition site, wherein the recognition site was not a sequence that would have been recognized by a native (non-engineered or non-modified) nuclease agent. Producing a nick or double-strand break in a recognition site or other DNA can be referred to herein as “cutting” or “cleaving” the recognition site or other DNA.

“Coding sequence” or “encoding nucleic acid” as used herein means the nucleic acids (RNA or DNA molecule) that comprise a nucleotide sequence which encodes a protein, e.g., a Cas9 protein, a CAR, or a TCR, or a polynucleotide, e.g., a gRNA. The coding sequence can further include initiation and termination signals operably linked to regulatory elements including a promoter and polyadenylation signal capable of directing expression in the cells of an individual or mammal to which the nucleic acid is administered. The coding sequence can be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

Various aspects described herein are described in further detail in the following subsections.

### II. Modified Immune Cells

Success of cellular immunotherapy for solid tumors has been limited due to exhaustion of tumor-infiltrating lymphocyte (TIL) in the tumor microenvironment. Persistent exposure to tumor antigens leads to T cell exhaustion, which is characterized by progressive loss of cytotoxicity and cytokine production, and increased expressions of inhibitory markers such as PD-1 (Wherry et al., Nat. Rev. Immunol. 15, 486-499 (2015)). Additionally, exhausted TILs upregulate and alternatively use transcription factors, particularly the NR4A family (Chen et al., Nature 567, 530-534 (2019)).

The present disclosure provides modified immune cells, i.e., cells modified, e.g., by gene editing, which express reduced levels of NR4A3 gene and/or NR4A3 protein, and as a result display an enhanced function, e.g., persistent effector function and/or reduced exhaustion (e.g., CAR T cells which express reduced levels of NR4A3 but endogenous levels of NR4A1 and NR4A2, e.g., anti-ROR1-CAR T cells which express reduced levels of NR4A3 but endogenous levels of NR4A1 and NR4A2, e.g., TCR expressing immune cells which express reduced level of NR4A3 but endogenous levels of NR4A1 and NR4A2). In some aspects, such modified immune cells have been further modified to also express a reduced level of other members of the NR4A family. For example, as described herein, in some aspects, modified immune cells expressing reduced level of NR4A3 gene and/or NR4A3 protein have also been modified to express reduced level of NR4A2 gene and/or NR4A2 protein. In some aspects, modified immune cells expressing reduced level of NR4A3 gene and/or NR4A3 protein have also been modified to express reduced level of NR4A1 gene and/or NR4A1 protein. In some aspects, modified immune cells expressing reduced level of NR4A3 gene and/or NR4A3 protein have also been modified to express reduced level of both the NR4A1 gene and/or NR4A1 protein and the NR4A2 gene and/or NR4A2 protein.

### II.A. NR4A3

Nuclear Receptor Subfamily 4 Group A Member 3, generally abbreviated NR4A3, and also known as MINOR, CSMF, NOR1, CHN, Mitogen-Induced Nuclear Orphan Receptor, Neuron-Derived Orphan Receptor, Nuclear Hormone Receptor NOR-1, “Chondrosarcoma, Extraskeletal Myxoid, Fused to EWS,” and TEC, is a protein which in humans is encoded by the NR4A3 gene. The NR4A family of orphan nuclear receptors includes NR4A1 (Nur77), NR4A2 (Nurr1), and NR4A3 (Nor-1). They work as transcription factors in a ligand-independent manner. Their functions are mostly controlled by the rapid and transient induction of their expression by a variety of extracellular signals, and thus are considered as immediate-early genes. The NR4As are involved in various cellular functions including apoptosis, survival, proliferation, angiogenesis, inflammation, DNA repair, and fatty acid metabolism.

The NR4A3 gene is located on chromosome 9 (bases 99,821,885 to 99,866,893; 45,039 bases; plus strand orientation; NCBI Reference Sequence: NC_000009.12). NR4A3 is a transcriptional activator that binds to regulatory elements in promoter regions in a cell- and response element (target)-specific manner. NR4A3 induces gene expression by binding as monomers to the NR4A1 response element (NBRE) 5′-AAAAGGTCA-3′ site and as homodimers to the Nur response element (NurRE) site in the promoter of their regulated target genes (by similarity) and plays a role in the regulation of proliferation, survival, and differentiation of many different cell types.

The NR4A3 proteins have three isoforms produced by alternative splicing. The sequences are shown in the Table 1 below.

TABLE 1
NR4A3 protein isoforms.

| | |
|---|---|
| NR4A3 | MPCVQAQYSPSPPGSSYAAQTYSSEYTTEIMNPDYTKLTMDLGSTEITATATTSLPSIST |
| Isoform | FVEGYSSNYELKPSCVYQMQRPLIKVEEGRAPSYHHHHHHHHHHHHHHQQQHQQPSIPPA |
| Alpha | SSPEDEVLPSTSMYFKQSPPSTPTTPAFPPQAGALWDEALPSAPGCIAPGPLLDPPMKAV |
| (identifier: | PTVAGARFPLFHFKPSPPHPPAPSPAGGHHLGYDPTAAAALSLPLGAAAAAGSQAAALES |
| Q92570-1) | HPYGLPLAKRAAPLAFPPLGLTPSPTASSLLGESPSLPSPPSRSSSSGEGTCAVCGDNAA |
| (SEQ ID NO: 1) | CQHYGVRTCEGCKGFFKRTVQKNAKYVCLANKNCPVDKRRRNRCQYCRFQKCLSVGMVKE |
| | VVRTDSLKGRRGRLPSKPKSPLQQEPSQPSPPSPPICMMNALVRALTDSTPRDLDYSRYC |
| | PTDQAAAGTDAEHVQQFYNLLTASIDVSRSWAEKIPGFTDLPKEDQTLLIESAFLELFVL |
| | RLSIRSNTAEDKFVFCNGLVLHRLQCLRGFGEWLDSIKDFSLNLQSLNLDIQALACLSAL |
| | SMITERHGLKEPKRVEELCNKITSSLKDHQSKGQALEPTESKVLGALVELRKICTLGLQR |
| | IFYLKLEDLVSPPSIIDKLFLDTLPF |
| NR4A3 | MPCVQAQYSPSPPGSSYAAQTYSSEYTTEIMNPDYTKLTMDLGSTEITATATTSLPSIST |
| Isoform Beta | FVEGYSSNYELKPSCVYQMQRPLIKVEEGRAPSYHHHHHHHHHHHHHHQQQHQQPSIPPA |
| (identifier: | SSPEDEVLPSTSMYFKQSPPSTPTTPAFPPQAGALWDEALPSAPGCIAPGPLLDPPMKAV |
| Q92570-2) | PTVAGARFPLFHFKPSPPHPPAPSPAGGHHLGYDPTAAAALSLPLGAAAAAGSQAAALES |
| (SEQ ID NO: 2) | HPYGLPLAKRAAPLAFPPLGLTPSPTASSLLGESPSLPSPPSRSSSSGEGTCAVCGDNAA |
| | CQHYGVRTCEGCKGFFKRTVQKNAKYVCLANKNCPVDKRRRNRCQYCRFQKCLSVGMVKE |
| | VVRTDSLKGRRGRLPSKPKSPLQQEPSQPSPPSPPICMMNALVRALTDSTPRDLDYSRVS |
| | FMISCFQMNDQGLYLWLLVIRVD |
| NR4A3 | MHDSIRFGNVDMPCVQAQYSPSPPGSSYAAQTYSSEYTTEIMNPDYTKLTMDLGSTEITA |
| Isoform 3 | TATTSLPSISTFVEGYSSNYELKPSCVYQMQRPLIKVEEGRAPSYHHHHHHHHHHHHHHQ |
| (identifier: | QQHQQPSIPPASSPEDEVLPSTSMYFKQSPPSTPTTPAFPPQAGALWDEALPSAPGCIAP |
| Q92570-3) | GPLLDPPMKAVPTVAGARFPLFHFKPSPPHPPAPSPAGGHHLGYDPTAAAALSLPLGAAA |
| (SEQ ID NO: 3) | AAGSQAAALESHPYGLPLAKRAAPLAFPPLGLTPSPTASSLLGESPSLPSPPSRSSSSGE |
| | GTCAVCGDNAACQHYGVRTCEGCKGFFKRTVQKNAKYVCLANKNCPVDKRRRNRCQYCRF |
| | QKCLSVGMVKEVVRTDSLKGRRGRLPSKPKSPLQQEPSQPSPPSPPICMMNALVRALTDS |
| | TPRDLDYSRYCPTDQAAAGTDAEHVQQFYNLLTASIDVSRSWAEKIPGFTDLPKEDQTLL |
| | IESAFLELFVLRLSIRSNTAEDKFVFCNGLVLHRLQCLRGFGEWLDSIKDFSLNLQSLNL |
| | DIQALACLSALSMITERHGLKEPKRVEELCNKITSSLKDHQSKGQALEPTESKVLGALVE |
| | LRKICTLGLQRIFYLKLEDLVSPPSIIDKLFLDTLPF |

In addition to reduced level of a NR4A3 gene and/or NR4A3 protein, in some aspects, a modified cell described herein can be further modified to have a reduced level of a NR4A2 gene and/or NR4A2 protein. Any suitable methods known in the art can be used to reduce the level of a NR4A2 gene and/or NR4A2 protein in the modified cells described herein. For example, in some aspects, the level of a NR4A2 gene and/or NR4A2 protein can be reduced using any of the gene editing tools described herein (e.g., CRISPR/Cas system).

Nuclear receptor subfamily 4 group A member 2, generally abbreviated NR4A2, also known as NOT, RNTR1, HZF-3, NURR1, TINUR, is a protein which in humans is encoded by the NR4A2 gene. The NR4A2 gene is located on chromosome 2 (bases 156,324,432 to 156,332,724, NCBI Reference Sequence: NC_000002.12). Unless indicated otherwise, the term “NR4A2 gene” used herein refers to any nucleic acid sequences encoding a NR4A2 protein (or variants thereof).

The NR4A2 protein has two isoforms produced by alternative splicing. The sequences are shown in Table 2 below. Unless indicated otherwise and as further described herein, in some aspects, an immune cell described herein has been further modified to have reduced level of any known NR4A2 protein (including any isoforms and variants thereof). Suitable methods of reducing the level of a NR4A2 gene and/or NR4A2 protein are described elsewhere in the present disclosure and also known in the art.

TABLE 2
NR4A2 protein isoforms.

| | |
|---|---|
| NR4A2 | MPCVQAQYGSSPQGASPASQSYSYHSSGEYSSDFLTPEFVKFSMDLTNTEITATTSLPSFSTFMD |
| Isoform 1 | NYSTGYDVKPPCLYQMPLSGQQSSIKVEDIQMHNYQQHSHLPPQSEEMMPHSGSVYYKPSSPPTP |
| (identifier: | TTPGFQVQHSPMWDDPGSLHNFHQNYVATTHMIEQRKTPVSRLSLFSFKQSPPGTPVSSCQMRFD |
| P43354-1) | GPLHVPMNPEPAGSHHVVDGQTFAVPNPIRKPASMGFPGLQIGHASQLLDTQVPSPPSRGSPSNE |
| (SEQ ID NO: 43) | GLCAVCGDNAACQHYGVRTCEGCKGFFKRTVQKNAKYVCLANKNCPVDKRRRNRCQYCRFQKCLA |
| | VGMVKEVVRTDSLKGRRGRLPSKPKSPQEPSPPSPPVSLISALVRAHVDSNPAMTSLDYSRFQAN |
| | PDYQMSGDDTQHIQQFYDLLTGSMEIIRGWAEKIPGFADLPKADQDLLFESAFLELFVLRLAYRS |
| | NPVEGKLIFCNGVVLHRLQCVRGFGEWIDSIVEFSSNLQNMNIDISAFSCIAALAMVTERHGLKE |
| | PKRVEELQNKIVNCLKDHVTFNNGGLNRPNYLSKLLGKLPELRTLCTQGLQRIFYLKLEDLVPPP |
| | AIIDKLFLDTLPF |
| NR4A2 | MDNYSTGYDVKPPCLYQMPLSGQQSSIKVEDIQMHNYQQHSHLPPQSEEMMPHSGSVYYKPSSPP |
| Isoform 2 | TPTTPGFQVQHSPMWDDPGSLHNFHQNYVATTHMIEQRKTPVSRLSLFSFKQSPPGTPVSSCQMR |
| (identifier: | FDGPLHVPMNPEPAGSHHVVDGQTFAVPNPIRKPASMGFPGLQIGHASQLLDTQVPSPPSRGSPS |
| P43354-2) | NEGLCAVCGDNAACQHYGVRTCEGCKGFFKRTVQKNAKYVCLANKNCPVDKRRRNRCQYCRFQKC |
| (SEQ ID NO: 44) | LAVGMVKEVVRTDSLKGRRGRLPSKPKSPQEPSPPSPPVSLISALVRAHVDSNPAMTSLDYSRFQ |
| | ANPDYQMSGDDTQHIQQFYDLLTGSMEIIRGWAEKIPGFADLPKADQDLLFESAFLELFVLRLAY |
| | RSNPVEGKLIFCNGVVLHRLQCVRGFGEWIDSIVEFSSNLQNMNIDISAFSCIAALAMVTERHGL |
| | KEPKRVEELQNKIVNCLKDHVTFNNGGLNRPNYLSKLLGKLPELRTLCTQGLQRIFYLKLEDLVP |
| | PPAIIDKLFLDTLPF |

### II.C. NR4A1

In addition to reduced level of a NR4A3 gene and/or NR4A3 protein, in some aspects, a modified cell described herein can be further modified to have a reduced level of a NR4A1 gene and/or NR4A1 protein. Any suitable methods known in the art can be used to reduce the level of a NR4A1 gene and/or NR4A1 protein in the modified cells described herein. For example, in some aspects, the level of a NR4A1 gene and/or NR4A1 protein can be reduced using any of the gene editing tools described herein (e.g., CRISPR/Cas system).

Nuclear Receptor Subfamily 4 Group A Member 1, generally abbreviated NR4A1, and also known as HMR, N10, TR3, NP10, GFRP1, NAK-1, NGFIB, and NUR77, is a protein which in humans is encoded by the NR4A1 gene. The NR4A1 gene is located on chromosome 12 (bases 52022832 to 52059507; NCBI Reference Sequence NC_000012.12). Unless indicated otherwise, the term “NR4A1 gene” used herein refers to any nucleic acid sequences encoding a NR4A1 protein (or variants thereof).

The NR4A1 protein has three isoforms produced by alternative splicing. The sequences are shown in Table 3 below.

TABLE 3
NR4A1 protein isoforms.

| | |
|---|---|
| NR4A1 | MPCIQAQYGTPAPSPGPRDHLASDPLTPEFIKPTMDLASPEAAPAAPTALPSFSTFMDGYTGEFD |
| Isoform 1 | TFLYQLPGTVQPCSSASSSASSTSSSSATSPASASFKFEDFQVYGCYPGPLSGPVDEALSSSGSD |
| (identifier: | YYGSPCSAPSPSTPSFQPPQLSPWDGSFGHFSPSQTYEGLRAWTEQLPKASGPPQPPAFFSFSPP |
| P22736-1) | TGPSPSLAQSPLKLFPSQATHQLGEGESYSMPTAFPGLAPTSPHLEGSGILDTPVTSTKARSGAP |
| (SEQ ID NO: 45) | GGSEGRCAVCGDNASCQHYGVRTCEGCKGFFKRTVQKNAKYICLANKDCPVDKRRRNRCQFCRFQ |
| | KCLAVGMVKEVVRTDSLKGRRGRLPSKPKQPPDASPANLLTSLVRAHLDSGPSTAKLDYSKFQEL |
| | VLPHFGKEDAGDVQQFYDLLSGSLEVIRKWAEKIPGFAELSPADQDLLLESAFLELFILRLAYRS |
| | KPGEGKLIFCSGLVLHRLQCARGFGDWIDSILAFSRSLHSLLVDVPAFACLSALVLITDRHGLQE |
| | PRRVEELQNRIASCLKEHVAAVAGEPQPASCLSRLLGKLPELRTLCTQGLQRIFYLKLEDLVPPP |
| | PIIDKIFMDTLPF |
| NR4A1 | MWLAKACWSIQSEMPCIQAQYGTPAPSPGPRDHLASDPLTPEFIKPTMDLASPEAAPAAPTALPS |
| Isoform 2 | FSTFMDGYTGEFDTFLYQLPGTVQPCSSASSSASSTSSSSATSPASASFKFEDFQVYGCYPGPLS |
| (identifier: | GPVDEALSSSGSDYYGSPCSAPSPSTPSFQPPQLSPWDGSFGHFSPSQTYEGLRAWTEQLPKASG |
| P22736-2) | PPQPPAFFSFSPPTGPSPSLAQSPLKLFPSQATHQLGEGESYSMPTAFPGLAPTSPHLEGSGILD |
| (SEQ ID NO: 46) | TPVTSTKARSGAPGGSEGRCAVCGDNASCQHYGVRTCEGCKGFFKRTVQKNAKYICLANKDCPVD |
| | KRRRNRCQFCRFQKCLAVGMVKEVVRTDSLKGRRGRLPSKPKQPPDASPANLLTSLVRAHLDSGP |
| | STAKLDYSKFQELVLPHFGKEDAGDVQQFYDLLSGSLEVIRKWAEKIPGFAELSPADQDLLLESA |
| | FLELFILRLAYRSKPGEGKLIFCSGLVLHRLQCARGFGDWIDSILAFSRSLHSLLVDVPAFACLS |
| | ALVLITDRHGLQEPRRVEELQNRIASCLKEHVAAVAGEPQPASCLSRLLGKLPELRTLCTQGLQR |
| | IFYLKLEDLVPPPPIIDKIFMDTLPF |
| NR4A1 | MPCIQAQYGTPAPSPGPRDHLASDPLTPEFIKPTMDLASPEAAPAAPTALPSFSTFMDGYTGEFD |
| Isoform 3 | TFLYQLPGTVQPCSSASSSASSTSSSSATSPASASFKFEDFQVYGCYPGPLSGPVDEALSSSGSD |
| (identifier: | YYGSPCSAPSPSTPSFQPPQLSPWDGSFGHFSPSQTYEGLRAWTEQLPKASGPPQPPAFFSFSPP |
| P22736-3) | TGPSPSLAQSPLKLFPSQATHQLGEGESYSMPTAFPGLAPTSPHLEGSGILDTPVTSTKARSGAP |
| (SEQ ID NO: 47) | GGSEGRCAVCGDNASCQHYGVRTCEGCKGFFKVPRSPRWGLLLEMERGWPHPIGTCGLPLGSPPS |

In some aspects, the cell composition useful for the present disclosure comprises a population of modified immune cells that express reduced levels of NR4A3 gene and/or reduced levels of NR4A3 protein and a binding molecule that specifically binds to ROR1 and that have: (i) endogenous expression of NR4A1 and NR4A2 genes and NR4A1 and NR4A2 proteins. In some aspects, the modified immune cells: (i) express a binding molecule that specific binds to ROR1; (ii) have a reduced level of NR4A3 gene and/or NR4A3 protein; and (iii) have a reduced level of NR4A1 gene and/or NR4A1 protein. In some aspects, the modified immune cells: (i) express a binding molecule that specific binds to ROR1; (ii) have a reduced level of NR4A3 gene and/or NR4A3 protein; and (iii) have a reduced level of NR4A2 gene and/or NR4A2 protein. In some aspects, the modified immune cells: (i) express a binding molecule that specific binds to ROR1; (ii) have a reduced level of NR4A3 gene and/or NR4A3 protein; (iii) have a reduced level of NR4A1 gene and/or NR4A1 protein; and (iv) have a reduced level of NR4A2 gene and/or NR4A2 protein. As used herein, the term “NR4A3 gene” refers to any transcript, genomic DNA, pre-mRNA, or mRNA. As used herein, the term “NR4A3 protein” refers to isoform alpha, isoform beta, or isoform 3 disclosed above, as well as variants and mutants thereof. As used herein, the term NR4A3 protein also encompasses any fragment or variant of any of the isoforms disclosed herein that has at least one function of the wild type NR4A3 protein.

As used herein the term “reduced levels” (or variant thereof) refers both to reduction in physical levels (e.g., less gene sequence due to edition from the genome, or less protein due a decrease in protein expression) and to reduction in function. For example, a reduction in level of NR4A3 gene can refer to a decrease in gene function, e.g., due to the introduction of a mutation introducing a stop codon or a frame shift, to an epigenetic modification that would alter transcription, or to a mutation or other change on a promoter gene or another gene that regulates NR4A3 expression. In some aspects, a reduction in level of NR4A3 gene in a modified cell refers to a decrease in the amount (e.g., concentration) of genomic DNA, pre-mRNA, and/or mRNA that is capable of encoding a functional NR4A3 protein, e.g., wild type NR4A3 protein, compared to a reference cell. Similarly, a reduction in NR4A3 protein can refer to changes resulting in the expression of a functional NR4A3 protein, e.g., wild type NR4A3 protein, including but not limited to changes (e.g., mutations or post-translational modifications) that cause a loss of function (partial or complete), or to the activity of molecules that bind to functional sites of NR4A3 altering, e.g., its interaction with other cell signaling partners.

NR4A3 gene levels (e.g., presence/absence of the entire gene or a portion thereof, or gene function) can be measured by various methods known in the art. NR4A3 protein levels (e.g., presence/absence of the NR4A3 protein or fragments thereof, or quantification or protein function) can be measured by various methods known in the art.

In some aspects, the expression levels of NR4A3 gene and/or expression levels of NR4A3 protein in the population of immune cells (e.g., CAR or TCR-expressing cells) is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells, e.g., corresponding cells that have not been modified to express lower levels of NR4A3 gene and/or NR4A3 protein. In some aspects, the expression of NR4A3 gene and/or NR4A3 protein in protein in the population of immune cells (e.g., a population of CAR-expressing cells or a TCR-expressing cells) is completely inhibited after the modification.

In some aspects, the expression level of NR4A3 gene in the population of immune cells is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells, e.g., corresponding cells that have not been modified to express lower levels of NR4A3 gene.

In some aspects, the expression level of NR4A3 protein in the population of immune cells is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells, e.g., corresponding cells that have not been modified to express lower levels of NR4A3 protein.

In some aspects, the expression levels of NR4A3 gene and NR4A3 protein in the population of immune cells are reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells, e.g., corresponding cells that have not been modified to express lower levels of NR4A3 gene and NR4A3 protein.

Similarly, in some aspects, modified immune cells disclosed herein (i.e., cells that expresses reduced levels of NR4A3 gene and/or NR4A3 protein) also has a level of NR4A1 gene and/or NR4A1 protein, which is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells (e.g., corresponding cells that have not been modified to express lower level of the NR4A1 gene and/or NR4A1 protein). In some aspects, modified immune cells disclosed herein (i.e., cells that expresses reduced levels of NR4A3 gene and/or NR4A3 protein) also has a level of NR4A2 gene and/or NR4A2 protein, which is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells (e.g., corresponding cells that have not been modified to express lower level of the NR4A2 gene and/or NR4A2 protein). In some aspects, modified immune cells disclosed herein (i.e., cells that expresses reduced levels of NR4A3 gene and/or NR4A3 protein) also has: (i) a level of NR4A1 gene and/or NR4A1 protein, which is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells (e.g., corresponding cells that have not been modified to express lower level of the NR4A1 gene and/or NR4A1 protein); and (ii) a level of NR4A2 gene and/or NR4A2 protein, which is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a population of reference immune cells (e.g., corresponding cells that have not been modified to express lower level of the NR4A2 gene and/or NR4A2 protein).

In some aspects, modified immune cells disclosed herein (i.e., cells that expresses reduced levels of NR4A3 gene and/or NR4A3 protein) comprise lymphocytes, neutrophils, monocytes, macrophages, dendritic cells, or combinations thereof. In some aspects, modified immune cells disclosed herein (i.e., a population of cells that expresses reduced levels of NR4A3 gene and/or NR4A3 protein) comprise lymphocytes. As used herein, “modified immune cells” include progeny cells of the originally modified immune cells, wherein the progeny cells also express reduced levels of NR4A3 gene and/or NR4A3 protein (alone or in combination with reduced level of the NR4A1 gene and/or NR4A1 protein and/or the NR4A2 gene and/or NR4A2 protein).

In some aspects, the population of immune cells is a pure population. In some aspects, the pure population comprises at least about 70%, at least about 75%, at least about 80%, at least about 85%, at least about 90%, at least about 95%, or at least 99% of cells belonging to the same immune cell type (e.g., 99% of the immune cells are lymphocytes). In some aspects, the population of immune cells comprises, one, two, three, four, or five different cell types, e.g., a population of immune cells comprising two cell types could comprise lymphocytes and dendritic cells.

In some aspects, a population of modified immune cells disclosed herein comprises, consists, or consists essentially of lymphocytes. In some aspects, a population of modified immune cells disclosed herein comprises lymphocytes, wherein the lymphocytes are selected from the group consisting of T cells, tumor-infiltrating lymphocytes (TIL), lymphokine-activated killer cells, natural killer (NK) T cells, and any combination thereof. In some specific aspects, the lymphocytes are T cells. In some specific aspects, the lymphocytes are NK cells.

In some aspects, a modified immune cell disclosed herein is a T cell. In some aspects, the T cell comprises a CAR. In some aspects, the modified T cell that can be prepared to express a CAR (a CAR T cell) is, e.g., a CD8⁺ T cell or CD4⁺ T cell. In some aspects, a CAR-expressing cell disclosed herein is a CAR T cell, e.g., a mono CAR T cell, a genome-edited CAR T cell, a dual CAR T cell, or a tandem CAR T cell. In some aspects, a modified cell disclosed herein is an NK cell. In some aspects, the NK cell comprises a CAR. In some aspects, the CAR NK cell is a mono CAR NK cell, a dual CAR NK cell, or a tandem CAR NKT cell. In some aspects, a modified cell of the presence disclosure comprises both T cells and NK cells. In some aspects, the T cells and NK cells both comprise CARs. Examples of such CAR T cells and CAR NK cells are provided in International Application No. PCT/US2019/044195 (published as WO2020028400A1), which is incorporated herein by reference in its entirety.

In some aspects, the modified immune cell can be any immune cell type. In some aspects, the cells are modified immune cells for any adoptive cell transfer (ACT) therapy (also known as adoptive cell therapy). ACT therapy can be an autologous therapy or allogenic therapy. In some aspects, the ACT therapy includes, but are not limited to a CAR T therapy, a tumor-infiltrating lymphocyte (TIL) therapy, an NK cell therapy, or any combination thereof.

In some aspects, the modified immune cells are TILs for a TIL therapy. The use of TILs as an adoptive cell transfer therapy to treat cancer have been studied for more than two decades using TIL adoptive cell therapy for melanoma. Rosenberg S A et al., (July 2011). Clinical Cancer Research 17 (13): 4550-7 (July 2011). In adoptive T cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. TILs are expanded over the course of a few weeks. Selected TIL lines that presented best tumor reactivity are then further expanded in a “rapid expansion protocol” (REP), which uses anti-CD3 activation for a typical period of two weeks. The TILs grown in the culture can be modified any time during the ex vivo process so that the expression of NR4A3 gene and/or NR4A3 protein (alone or in combination with other members of the NR4A family, e.g., NR4A1 and/or NR4A2) is reduced. The final post-REP TIL is infused back into the patient. The process can also involve a preliminary chemotherapy regimen to deplete endogenous lymphocytes in order to provide the adoptively transferred TILs with enough access to surround the tumor sites.

In some aspects, a modified immune cell disclosed herein, e.g., a T cell, comprises a T cell receptor (TCR), e.g., an engineered T cell receptor. In some aspects, a modified immune cell disclosed herein, e.g., a T cell, can comprise a chimeric antigen receptor (CAR) that specifically binds to a tumor antigen. In some aspects, the modified immune cell, e.g., a lymphocyte, is T cell with T cell receptors, e.g., engineered TCRs. As used herein, the term “engineered TCR” or “engineered T cell receptor” refers to a T cell receptor (TCR) engineered to specifically bind with a desired affinity to a major histocompatibility complex (MHC)/peptide target antigen that is selected, cloned, and/or subsequently introduced into a population of T cells.

In some aspects, CARs or TCRs that can be expressed on a modified cell disclosed herein specifically bind (i.e., target) one or more antigens expressed on a tumor cell, such as a malignant B cell, a malignant T cell, or a malignant plasma cell.

In some aspects, the CAR specifically binds to (i.e., targets) an antigen selected from the group consisting of CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-l lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, ber-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTl, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTl, Ras mutant (e.g., including KRAS, HRAS, NRAS mutant proteins), hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In some aspects, a modified cell of the present disclosure can express a T cell receptor (TCR) targeting an antigen. T cell receptor is a heterodimer composed of 2 different transmembrane polypeptide chains: an α chain and a β chain, each consisting of a constant region, which anchors the chain inside the T cell surface membrane, and a variable region, which recognizes and binds to the antigen presented by MHCs. The TCR complex is associated with 6 polypeptides forming 2 heterodimers, CD37γε and CD36δε, and 1 homodimer CD3 ζ, which together forms the CD3 complex. T cell receptor-engineered T cell therapy utilizes the modification of T cells that retain these complexes to specifically target the antigens expressed by particular tumor cells.

In some aspects, the modified TCR engineered cells can target main types: shared tumor-associated antigens (shared TAAs) and unique tumor-associated antigens (unique TAAs), or tumor-specific antigens. The former can include, without any limitation, cancer-testis (CT) antigens, overexpressed antigens, and differentiation antigens, while the latter can include, without any limitation, neoantigens and oncoviral antigens. Human papillomavirus (HPV) E6 protein and HPV E7 protein belong to the category of oncoviral antigens.

In some aspects, the modified TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the modified TCR engineered cells can target glycoprotein (gp100), melanoma antigen recognized by T cells (MART-1), and/or tyrosinase, which are mainly found in melanomas and normal melanocytes. In some aspects, the modified TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the modified TCR engineered cells can target mesothelin, another kind of overexpressed antigen that is highly expressed in mesothelioma but is also present on mesothelial cells of several tissues, including trachea.

In some aspects, the modified TCR engineered cells can target any neoantigen, which can be formed by random somatic mutations specific to individual tumors. In some aspects, the TCR specifically binds to (i.e., targets) a cancer antigen selected from the group consisting of AFP, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-l lRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTl, NY-ESO-1, LAGE-la, MAGE-Al, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTl, Ras mutant (e.g., including KRAS, HRAS, NRAS mutant proteins), hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, and any combinations thereof.

In some aspects, a modified immune cell of the present disclosure, e.g., a CAR T or NK cell or a TCR-engineered T cell, can target any one of the tumor antigens disclosed above or a combination thereof. Receptor tyrosine kinase-like orphan receptor 1 (ROR1) is overexpressed in approximately 57% of patients with triple negative breast cancer (TNBC) and 42% of patients with non-small cell lung carcinoma (NSCLC) adenocarcinomas (Balakrishnan 2017) and represents a highly attractive target for chimeric antigen receptor (CAR) T cells. Receptor tyrosine kinase-like orphan receptor 1-positive (ROR1⁺) solid tumors can be safely targeted with anti-ROR1 CAR T cells (Specht 2020); however, efficacy has been limited, in part, because the CAR T cells exhibit exhaustion or dysfunction following infusion in patients with solid-tumor malignancies. In addition, solid tumors have immune-suppressive barriers that limit antitumor activity of immunotherapies, such as CAR T cells (Newick 2016, Srivastava 2018, Martinez 2019). Without wishing to be bound by any one theory, cells expressing the anti-ROR1 chimeric binding proteins described herein have been modified to express reduced levels of NR4A3 gene and/or NR4A3 protein and that have endogenous expression of NR4A1 and NR4A2 genes and NR4A1 and NR4A2 proteins. And, as described and demonstrated herein, in some aspects, such cells expressing the anti-ROR1 chimeric binding protein and expressing reduced level of NR4A3 gene and/or NR4A3 protein have been further modified to express reduced level of NR4A1 gene and/or NR4A1 protein. In some aspects, such cells (i.e., expressing the anti-ROR1 chimeric binding protein and expressing reduced level of NR4A3 gene and/or NR4A3 protein) have been further modified to express reduced level of NR4A2 gene and/or NR4A2 protein. In some aspects, such cells (i.e., expressing the anti-ROR1 chimeric binding protein and expressing reduced level of NR4A3 gene and/or NR4A3 protein) have been further modified to express both reduced level of NR4A1 gene and/or NR4A1 protein and reduced level of NR4A2 gene and/or NR4A2 protein. These modified cells are more resistant to exhaustion and exhibit improved effector functions compared to other anti-ROR1 cells available in the art.

In some aspects, the modified immune cells comprise a ROR1 binding chimeric antigen receptor. An exemplary anti-ROR1 CAR is described in Hudecek, et al. Clin. Cancer Res. 19.12(2013):3153-64, incorporated herein by reference in its entirety. In some aspects, a CAR T cell of the present disclosure comprising an anti-ROR1 CAR is generated as described in Hudecek et al. (for example, as described in Hudecek et al. at page 3155, first full paragraph, incorporated herein by reference in its entirety). In some aspect, an anti-ROR1 CAR of the present disclosure includes an antibody or fragment thereof comprising the VH and/or VL sequences of the 2A2, R11, and R12 anti-ROR1 monoclonal antibodies described in Hudecek et al. at paragraph bridging pages 3154-55; Baskar et al. MAbs 4(2012):349-61; and Yang et al. PLoS ONE 6(2011):e21018, incorporated herein by reference in their entirety.

In some aspects, an antigen-binding domain of the present disclosure is capable of cross-competing with an anti-ROR1 antibody, e.g., R12, antibody. The R12 antibody sequences are shown in TABLE 2. In some aspects, the antigen-binding domain useful for the present disclosure binds to the same epitope of the R12 antibody. As will be apparent to those skilled in the arts, any anti-ROR1 antibody known in the art can be used with the present disclosure. Non-limiting examples of such antibodies include the 2A2 and R11 antibodies described in Hudecek, et al. Clin. Cancer Res. 19.12(2013):3153-64; Baskar et al. MAbs 4(2012):349-61; and Yang et al. PLoS ONE 6(2011):e21018; U.S. Pat. No. 9,316,646 B2; and U.S. Pat. No. 9,758,586 B2; each of which is incorporated herein by reference in its entirety.

TABLE 2
R12 antibody CDRs and heavy chain variable region/light chain variable region

| | |
|---|---|
| R12 VH | QEQLVESGGRLVTPGGSLTLSCKASGFDFSAYYMSWVRQAPGKGLEWIATIYPSSGKTYYAT |
| (SEQ ID NO: 17) | WVNGRFTISSDNAQNTVDLQMNSLTAADRATYFCARDSYADDGALFNIWGPGTLVTISS |
| R12 VH CDR1 | AYYMS |
| (SEQ ID NO: 18) | |
| R12 VH CDR2 | TIYPSSGKTYYATWVNG |
| (SEQ ID NO: 19) | |
| R12 VH CDR3 | DSYADDGALFNI |
| (SEQ ID NO: 20) | |
| R12 VL | ELVLTQSPSVSAALGSPAKITCTLSSAHKTDTIDWYQQLQGEAPRYLMQVQSDGSYTKRPGV |
| (SEQ ID NO: 21) | PDRFSGSSSGADRYLIIPSVQADDEADYYCGADYIGGYVFGGGTQLTVTG |
| R12 VL CDR1 | TLSSAHKTDTID |
| (SEQ ID NO: 22) | |
| R12 VL CDR2 | GSYTKRP |
| (SEQ ID NO: 23) | |
| R12 VL CDR3 | GADYIGGYV |
| (SEQ ID NO: 24) | |

In some aspects, the antigen-binding domain of the CARs of the modified immune cells disclosed herein comprises VH CDR3 of the R12 antibody. In some aspects, the antigen-binding domain of the present disclosure comprises VH CDR1, VH CDR2 and VH CDR3 of the R12 antibody. In some aspects, the antigen-binding domain of the present disclosure comprises VH CDR1, VH CDR2, VH CDR3, VL CDR1, VL CDR2, and VL CDR3 of the R12 antibody. In some aspects, the antigen-binding domain of the present disclosure, e.g., R12 scFv, comprises the VH and the VL of the R12 antibody.

In some aspects, the intracellular domain of a chimeric binding protein (e.g., ROR1 CAR) of the modified immune cells disclosed herein comprises a signaling domain, such as that derived from CD3zeta, FcR gamma, FcR beta, CD3 gamma, CD3 delta, CD3 epsilon, CD5, CD22, CD79a, CD79b, or CD66d. In some aspect, the CAR further comprises a co-stimulatory domain, such as that derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, or B7-H3. In some aspects, the CAR comprises a 4-1BB costimulatory domain.

In some aspects, a transmembrane domain of a chimeric binding protein (e.g., CAR) of the modified immune cells disclosed herein can include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some aspects, the chimeric binding protein (e.g., CAR) of the modified immune cells disclosed herein further comprises a sequence encoding a costimulatory domain, e.g., a costimulatory domain described herein. In some aspects, the costimulatory domain comprises a costimulatory domain of interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, B7-H3, CD28 deleted for Lck binding (ICA), BTLA, GITR, HVEM, LFA-1, LIGHT, NKG2C, PD-1, TILR2, TILR4, TILR7, TILR9, Fc receptor gamma chain, Fc receptor F chain, a ligand that specifically binds with CD83, or any combination thereof.

In some aspects, the immune cells are modified by a gene editing tool to reduce the expression of the NR4A3 gene and/or NR4A3 protein (alone or in combination with reduced level of the NR4A1 gene and/or NR4A1 protein and/or the NR4A2 gene and/or NR4A2 protein). The reduced expression of the NR4A3 gene can be done, e.g., by editing the entire NR4A3 gene, by editing a portion of the NR4A3 gene, by editing a regulatory regions controlling the expression of the NR4A3 gene. Accordingly, to reduce the expression of NR4A3 gene and/or NR4A3 protein in a CAR-expressing cell or a TCR-expressing cell, any methods known in the art for reducing the expression of a gene and/or protein in a cell can be used. For instance, in some aspects, the expression of NR4A3 gene, and the NR4A3 protein encoded thereof, of a CAR-expressing cell or a TCR-expressing cell can be reduced by contacting the cell with a gene editing tool that is capable of reducing the expression levels of the NR4A3 gene, and the NR4A3 protein encoded thereof. Non-limiting examples of the gene editing tool are shown below. In some specific aspects, the gene editing tool comprises, e.g., a shRNA, siRNA, miRNA, antisense oligonucleotides, CRISPR, zinc finger nuclease, TALEN, meganuclease, restriction endonuclease, or any combination thereof. In a particular aspect, the gene editing tool is CRISPR. Wherein the expression of the NR4A2 gene and/or NR4A2 protein and/or the expression of the NR4A1 gene and/or NR4A1 protein are also reduced, in some aspects, such gene editing tools can also be used (e.g., specifically targeting the NR4A2 gene and/or the NR4A1 gene).

In some aspects, the population of immune cells (e.g., CAR or TCR-expressing cells produced by the methods disclosed herein, i.e., expressing reduced levels of NR4A3 gene and/or NR4A3 protein, alone or in combination with reduced level of the NR4A1 gene and/or NR4A1 protein and/or the NR4A2 gene and/or NR4A2 protein) exhibits one or more enhanced or improved properties of the immune cells in the subject compared to the reference immune cells (i.e., immune cells that have not been modified to have reduced NR4A3 active gene levels and/or a reduced NR4A3 protein expression levels). In some aspects, improving one or more properties of immune cells disclosed herein can help treat a tumor (e.g., reduce tumor volume and/or tumor weight). The one or more properties that can be improved with the present disclosure include any properties of an immune cell disclosed herein that can be useful in treating cancers. For example, in some aspects, the population of immune cells (e.g., CAR or TCR-expressing cells produced by the methods disclosed herein, i.e., expressing reduced levels of NR4A3 gene and/or NR4A3 protein, alone or in combination with reduced level of the NR4A1 gene and/or NR4A1 protein and/or the NR4A2 gene and/or NR4A2 protein) can exhibit greater effector activity compared to a reference cell (e.g., CAR or TCR-expressing cell that has not been modified to express lower levels of NR4A3 gene and/or NR4A3 protein).

In some aspects, the enhanced properties of the modified immune cells comprise (i) increased expansion and/or proliferation of the immune cells,
(ii) increased cytotoxicity of the immune cells,
(iii) increased cytokine expression of the immune cells, or
(iv) any combination thereof,
with respect to reference cells.

In some aspects, the modified immune cells disclosed herein (e.g., CAR or TCR-expressing cells described herein) are exhaustion-resistant and/or dysfunction-resistant compared to reference immune cells (i.e., immune cells that have not been modified to have reduced NR4A3 gene levels and/or a reduced NR4A3 protein expression levels).

In some aspects, the modified immune cells disclosed herein (e.g., CAR or TCR-expressing cells described herein) are apoptosis-resistant, i.e., they exhibit reduced or no apoptosis compared to reference immune cells (i.e., immune cells that have not been modified to have reduced NR4A3 gene levels and/or a reduced NR4A3 protein expression levels).

In some aspects, the modified immune cells disclosed herein (e.g., CAR or TCR-expressing cells described herein) are immune checkpoint-resistant, i.e., they exhibit reduced or no immune checkpoint activity compared to reference immune cells (i.e., immune cells that have not been modified to have reduced NR4A3 gene levels and/or a reduced NR4A3 protein expression levels).

In some aspects, the modified immune cells disclosed herein (e.g., CAR or TCR-expressing cells described herein) exhibit enhanced T cell activation compared to reference immune cells (i.e., immune cells that have not been modified to have reduced NR4A3 gene levels and/or a reduced NR4A3 protein expression levels). In some aspects, such enhanced T cell activation can be evidenced, e.g., by the modified immune cells exhibiting enhanced expansion, enhanced cytotoxicity, enhanced cytokine expression, or any combination thereof compared to reference immune cells (i.e., immune cells that have not been modified to have reduced NR4A3 gene levels and/or a reduced NR4A3 protein expression levels).

In some aspects, the modified immune cells disclosed herein (e.g., CAR or TCR-expressing cells described herein) maintain an anti-tumor function in a tumor microenvironment (TME) compared to reference immune cells (i.e., immune cells that have not been modified to have reduced NR4A3 gene levels and/or a reduced NR4A3 protein expression levels).

The present disclosure also provides pharmaceutical compositions comprising the modified immune cell populations disclosed herein and a pharmaceutical acceptable carrier.

### III. Methods of Treatment

Provided herein are methods for treating a tumor (or a cancer) in a subject in need thereof, comprising administering to the subject a cell composition of the disclosure, e.g., a cell expressing reduced levels of NR4A3 gene and/or the protein encoded thereof, i.e., NR4A3 protein and a binding molecule that specifically binds to ROR1 and that have: (i) endogenous expression of NR4A1 and NR4A2 genes and NR4A1 and NR4A2 proteins, (ii) reduced level of NR4A1 gene and/or NR4A1 protein, (iii) reduced level of NR4A2 gene and/or NR4A2 protein, or (iv) reduced level of both the NR4A1 gene and/or NR4A1 protein and NR4A2 gene and/or NR4A2 protein, or a pharmaceutical composition of the present disclosure.

In some aspects, the expression level of the NR4A3 gene is reduced by at least about 5%, at least about 10%, at least about 15%, at least about 30%, at least about 40%, at least about 50%, at least about 60%, at least about 70%, at least about 80%, at least about 90%, or at least about 100%, compared to a reference cell (e.g., corresponding cell that has not been modified to express lower levels of the NR4A3 gene). In some aspects, the expression level of the NR4A3 protein is reduced by at least about 5%, at least about 10%, at least about 15%, at least about 30%, at least about 40%, at least about 50%, at least about 60%, at least about 70%, at least about 80%, at least about 90%, or at least about 100%, compared to a reference cell (e.g., corresponding cell that has not been modified to express lower levels of NR4A3 protein). In some aspects, the expression levels of both the NR4A3 gene and the NR4A3 protein are reduced by at least about 5%, at least about 10%, at least about 15%, at least about 30%, at least about 40%, at least about 50%, at least about 60%, at least about 70%, at least about 80%, at least about 90%, or at least about 100%, compared to a reference cell (e.g., corresponding cell that has not been modified to express lower levels of the NR4A3 gene and/or NR4A3 protein). Methods of reducing the expression level of the NR4A3 gene and/or NR4A3 protein are provided elsewhere in the present disclosure.

In some aspects, administering the cell composition of the disclosure reduces a tumor volume in the subject compared to a reference tumor volume. In some aspects, the reference tumor volume is the tumor volume in the subject prior to the administration of the modified cell. In some aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In some aspects, a modified cell disclosed herein can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the modified cell. In some aspects, the reference tumor weight is the tumor weight in a corresponding subject that did not receive the administration.

In some aspects, administering the cell composition of the disclosure to a subject, e.g., suffering from a tumor, can increase the number and/or percentage of TILs (e.g., CD4⁺ or CD8⁺) in a tumor and/or TME of the subject. In some aspects, the number and/or percentage of TILs in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the modified cell or the same subject prior to the administration of the modified cell).

In some aspects, administering the cell composition of the disclosure can reduce the number and/or percentage of regulatory T cells in a tumor and/or TME of a subject. In some aspects, the number and/or percentage of regulatory T cells in a tumor and/or TME is decreased by at least about 5%, at least about 10%, at least about 15%, at least about 30%, at least about 40%, at least about 50%, at least about 60%, at least about 70%, at least about 80%, at least about 90%, or about 100% compared to a reference (e.g., the corresponding number and/or percentage in a subject that did not receive an administration of the modified cell).

In some aspects, administering the cell composition of the disclosure can decrease the number and/or percentage of myeloid-derived suppressor cells (MDSCs) in the tumor and/or TME of a subject. In some aspects, the MDSCs are monocytic MDSCs (M-MDSCs). In some aspects, the MDSCs are polymorphonuclear MDSCs (PMN-MDSCs). In some aspects, the MDSCs comprise both M-MDSCs and PMN-MDSCs. In some aspects, the number and/or percentage of MDSCs in the tumor and/or TME is decreased by at least about 5%, at least about 10%, at least about 15%, at least about 30%, at least about 40%, at least about 50%, at least about 60%, at least about 70%, at least about 80%, at least about 90%, or about 100% compared to a reference (e.g., value in a corresponding subject that did not receive an administration of the modified cell).

In addition to the above, administering the cell composition of the disclosure can have other effects which are conducive for the treatment of a tumor. Such effects are described further below.

As described herein, the cell composition of the disclosure (i.e., expresses reduced levels of NR4A3 gene and/or NR4A3 protein and a binding molecule that specifically binds to ROR1 and that have: (i) endogenous expression of NR4A1 and NR4A2 genes and NR4A1 and NR4A2 proteins, (ii) reduced level of NR4A1 gene and/or NR4A1 protein, (iii) reduced level of NR4A2 gene and/or NR4A2 protein, or (iv) reduced level of both the NR4A1 gene and/or NR4A1 protein and NR4A2 gene and/or NR4A2 protein) can be used to treat a variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, cervical cancer, carcinoma, sarcoma, leukemia, lymphoma, myeloma, or a combination thereof. A comprehensive and non limiting list of cancer indications is provided in the Indication section of this application.

In some aspects, the cell composition of the disclosure can be used in combination with other therapeutic agents (e.g., anti-cancer agents and/or immunomodulating agents). Accordingly, in some aspects, a method of treating a tumor disclosed herein comprises administering the cell composition of the disclosure in combination with one or more additional therapeutic agents. In some aspects, the cell composition of the disclosure can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail in the Combination Treatments section of this application.

In some aspects, the cell composition of the disclosure is administered to the subject prior to or after the administration of the additional therapeutic agent. In some aspects, the cell composition of the disclosure is administered to the subject concurrently with the additional therapeutic agent. In some aspects, the cell composition of the disclosure and the additional therapeutic agent can be administered concurrently as a single composition in a pharmaceutically acceptable carrier. In some aspects, the cell composition of the disclosure and the additional therapeutic agent are administered concurrently as separate compositions.

In some aspects, a subject that can be treated with the present disclosure is a nonhuman animal such as a rat or a mouse. In some aspects, the subject that can be treated is a human.

In some aspects, treating a tumor, e.g., in a method disclosed herein, comprises enhancing the activation of a T cell (e.g., tumor-specific T cell). As used herein, the term “enhancing the activation of a T cell” refers to altering the cell signaling during activation to promote the retention of T cell memory.

Accordingly, in some aspects, the present disclosure relates to methods of enhancing the activation of a T cell by reducing the expression levels of NR4A3 gene and/or NR4A3 protein (alone or in combination with reduced level of the NR4A1 gene and/or NR4A1 protein and/or the NR4A2 gene and/or NR4A2 protein) in the cell. The activation status of a cell can be determined by any method known in the art, e.g., by analyzing one or more functional properties (e.g., proliferation, cytotoxicity, cytokine production) of the cell or by analyzing the phenotypic expression of the cell. In some aspects, enhancing the activation of a T cell (e.g., tumor-specific T cell) can result in one or more of the following improved properties in the cell: (i) enhanced expansion, (ii) enhanced cytotoxicity, (iii) enhanced cytokine expression, or (iv) any combination thereof.

In some aspects, enhancing the activation of a T cell (e.g., tumor-specific T cell) results in enhanced expansion of the cell. In some aspects, the expansion of the T cell is enhanced (i.e., increased) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., expansion of a corresponding T cell that has not been modified to express lower levels of NR4A3 gene and/or NR4A3 protein). In some aspects, the expansion of the T cells in increased by about 5, 10, 15, 20, 25, 30, 35, 40, 45, 50, 55, 60, 65, 70, 75, 80, 85, 90, 95, or by about 100 fold. In some aspects, the expansion of the T cells is increased by about 2 fold to about 100, 150, 200, 250, 300, 350, 400, 450, 500 fold or more. In some aspects, the T expansion of the immune cells, is increased by about 10 fold to about 500 fold, from about 20 fold to about 400 fold, from about 25 fold to about 250 fold, from about 10 fold to about 50 fold, from about 20 fold to about 300 fold. In some aspects, the enhanced expansion can result in an increase in the number of the modified T cell (i.e., expressing reduced levels of NR4A3 gene and/or NR4A3 protein), e.g., in a subject. In some aspects, the number of the modified T cell is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about²⁰⁰%, at least about²10%, at least 220%, at least about 230%, at least about 240%, at least about²50%, at least about 260%, at least about 270%, at least about 280%, at least about 290%, or at least about 300% or more compared to a reference (e.g., number of a corresponding T cell that has not been modified to express lower levels of NR4A3 gene and/or NR4A3 protein).

In some aspects, enhancing the activation of a T cell (e.g., tumor-specific T cell) results in enhanced cytotoxicity of the cell. As used herein, the term “cytotoxicity” refers to the ability of a cell composition of the disclosure (e.g., tumor-specific T cell) to attack and induce damage in a tumor cell. The cell composition of the disclosure (e.g., tumor-specific T cell) can attack and induce damage in a tumor cell by any method known in the art, such as by inducing apoptosis in a tumor cell through the release of cytotoxic molecules (e.g., perforin, granzymes, and granulysin) or through Fas-Fas ligand interaction. In some aspects, the cytotoxicity of the T cell is enhanced (i.e., increased) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about²⁰⁰%, at least about²10%, at least 220%, at least about 230%, at least about 240%, at least about 250%, at least about 260%, at least about 270%, at least about 280%, at least about 290%, or at least about 300% or more compared to a reference (e.g., cytotoxicity of a corresponding T cell that has not been modified to express lower levels of NR4A3 gene and/or NR4A3 protein). In some aspects, the cytotoxicity (or killing activity) of the T cells is increased by about 5, 10, 15, 20, 25, 30, 35, 40, 45, 50, 55, 60, 65, 70, 75, 80, 85, 90, 95, or by about 100 fold. In some aspects, the cytotoxicity of the T cells is increased by about 2 fold to about 100, 150, 200, 250, 300, 350, 400, 450, 500 fold or more. In some aspects, the cytotoxicity of the T cells, is increased by about 10 fold to about 500 fold, from about 20 fold to about 400 fold, from about 25 fold to about 250 fold, from about 10 fold to about 50 fold, from about 20 fold to about 300 fold, or more as compared to a reference (e.g., cytotoxicity of a corresponding T cell that has not been modified to express lower levels of NR4A3 gene and/or NR4A3 protein).

In some aspects, enhancing the activation of a T cell (e.g., tumor-specific T cell) results in enhanced cytokine expression in the cell. In some aspects, the cytokine expression is enhanced (i.e., increased) by at least about at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference (e.g., cytokine expression in a corresponding T cell that has not been modified to express lower levels of NR4A3 gene and/or NR4A3 protein). In some aspects, cytokine expression in the T cells is increased by about 5, 10, 15, 20, 25, 30, 35, 40, 45, 50, 55, 60, 65, 70, 75, 80, 85, 90, 95, or by about 100 fold. In some aspects, cytokine expression in the T cells is increased by about 2 fold to about 100, 150, 200, 250, 300, 350, 400, 450, 500 fold or more. In some aspects, the cytokine expression in the T cells, is increased by about 10 fold to about 500 fold, from about 20 fold to about 400 fold, from about 25 fold to about 250 fold, from about 10 fold to about 50 fold, from about 20 fold to about 300 fold, or more as compared to a reference (e.g., cytokine expression of a corresponding T cell that has not been modified to express lower levels of NR4A3 gene and/or NR4A3 protein). As used herein, the term “cytokine” refers to any cytokine that can be useful in the treatment of a cancer. Non-limiting examples of such cytokines include IFN-γ, TNF-α, IL-2, and any combination thereof.

In some aspects, the expansion and/or proliferation and/or cytotoxicity (or killing activity) and/or cytokine expression of the T cells is increased by about 5, 10, 15, 20, 25, 30, 35, 40, 45, 50, 55, 60, 65, 70, 75, 80, 85, 90, 95, or by about 100 fold. In some aspects, the expansion and/or proliferation and/or cytotoxicity (or killing activity) and/or cytokine expression of the T cells is increased by about 2 fold to about 100, 150, 200, 250, 300, 350, 400, 450, 500 fold or more. In some aspects, the expansion and/or proliferation and/or cytotoxicity (or killing activity) and/or cytokine expression of the T cells, is increased by about 10 fold to about 500 fold, from about 20 fold to about 400 fold, from about 25 fold to about 250 fold, from about 10 fold to about 50 fold, from about 20 fold to about 300 fold, or more as compared to a reference (e.g., the expansion and/or proliferation and/or cytotoxicity (or killing activity) and/or cytokine expression of a corresponding T cell that has not been modified to express lower levels of NR4A3 gene and/or NR4A3 protein). In some aspects, the expansion and/or proliferation of the immune cells, cytotoxicity of the immune cells, or cytokine expression of the immune cells, is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to reference cells (e.g., corresponding immune cells that have not been modified to express lower levels of NR4A3 gene and/or NR4A3 protein).

In some aspects, the modified immune cells according to the present disclosure exhibits increased cytokine expression with respect to reference cells. In some aspects, the cytokines are interleukin-2 (IL-2), interferon-7 (IFN-γ), tumor necrosis factor-α (TNF-α), or any combination thereof.

In some aspects, the expression level of IL-2 in the modified immune cells is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold, or more compared to the expression level of IL-2 in reference immune cells.

In some aspects, the expression level of IFN-γ in the modified immune cells is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold, or more compared to the expression level of IFN-γ in reference immune cells.

In some aspects, the expression level of TNF-α in the modified immune cells is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold, or more compared to the expression level of TNF-α in reference immune cells.

In some aspects, a CAR or TCR-expressing cell disclosed herein (i.e., expressing reduced levels of NR4A3 gene and/or NR4A3 protein, alone or in combination with reduced level of the NR4A1 gene and/or NR4A1 protein and/or the NR4A2 gene and/or NR4A2 protein) produces increased amounts of IL-2 when stimulated with an antigen, such as a cognate antigen (e.g., tumor antigen), e.g., sequential stimulation and/or chronic stimulation. In some aspects, the amount of IL-2 produced in the CAR or TCR-expressing cell is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., CAR or TCR-expressing cell that has not been modified to express lower levels of NR4A3 gene and/or NR4A3 protein).

In some aspects, a CAR or TCR-expressing cell disclosed herein (i.e., expressing reduced levels of NR4A3 gene and/or NR4A3 protein, alone or in combination with reduced level of the NR4A1 gene and/or NR4A1 protein and/or the NR4A2 gene and/or NR4A2 protein) produces increased amounts of IFN-γ when stimulated with an antigen, such as a cognate antigen (e.g., tumor antigen), e.g., sequential stimulation and/or chronic stimulation. In some aspects, the amount of IFN-γ produced in the CAR or TCR-expressing cell is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., CAR or TCR-expressing cell that has not been modified to express lower levels of NR4A3 gene and/or NR4A3 protein).

In some aspects, a CAR or TCR-expressing cell disclosed herein (i.e., expressing reduced levels of NR4A3 gene and/or NR4A3 protein, alone or in combination with reduced level of the NR4A1 gene and/or NR4A1 protein and/or the NR4A2 gene and/or NR4A2 protein) produces increased amounts of TNF-α when stimulated with an antigen, such as a cognate antigen (e.g., tumor antigen), e.g., sequential stimulation and/or chronic stimulation. In some aspects, the amount of TNF-α produced in the CAR or TCR-expressing cell is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., CAR or TCR-expressing cell that has not been modified to express lower levels of NR4A3 gene and/or NR4A3 protein).

In some aspects, the modified immune cells disclosed herein (e.g., CAR or TCR-expressing cells described herein) exhibit increased cell expansion and/or cell proliferation compared to reference immune cells (i.e., immune cells that have not been modified to have reduced NR4A3 active gene levels and/or a reduced NR4A3 protein expression levels). In some aspects, the cell expansion and/or cell proliferation in the modified immune cells is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to reference cells (e.g., CAR or TCR-expressing cells that have not been modified to express lower levels of NR4A3 gene and/or NR4A3 protein).

In some aspects, the modified immune cells disclosed herein (e.g., CAR or TCR-expressing cells described herein) exhibit increased persistence and/or survival compared to reference immune cells (i.e., immune cells that have not been modified to have reduced NR4A3 active gene levels and/or a reduced NR4A3 protein expression levels). In some aspects, the persistence of the T cells is increased by about 5, 10, 15, 20, 25, 30, 35, 40, 45, 50, 55, 60, 65, 70, 75, 80, 85, 90, 95, or by about 100 fold. In some aspects, the persistence of the T cells is increased by about 2 fold to about 100, 150, 200, 250, 300, 350, 400, 450, 500 fold or more. In some aspects, the persistence of the T cells, is increased by about 10 fold to about 500 fold, from about 20 fold to about 400 fold, from about 25 fold to about 250 fold, from about 10 fold to about 50 fold, from about 20 fold to about 300 fold, or more as compared to a reference (e.g., the presistence of a corresponding T cell that has not been modified to express lower levels of NR4A3 gene and/or NR4A3 protein). In some aspects, the persistence and/or survival in the modified immune cells is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to reference cells (e.g., CAR or TCR-expressing cells that have not been modified to express lower levels of NR4A3 gene and/or NR4A3 protein).

In some aspects, the modified immune cells disclosed herein (e.g., CAR or TCR-expressing cells described herein) exhibit increased anti-tumor activity compared to reference immune cells (i.e., immune cells that have not been modified to have reduced NR4A3 gene levels and/or a reduced NR4A3 protein expression levels). In some aspects, the anti-tumor activity in the modified immune cells is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to reference cells (e.g., CAR or TCR-expressing cells that have not been modified to express lower levels of NR4A3 gene and/or NR4A3 protein).

In some aspects, the modified immune cells disclosed herein (e.g., CAR or TCR-expressing cells described herein) exhibit reduced exhaustion or dysfunction compared to reference immune cells (i.e., immune cells that have not been modified to have reduced NR4A3 gene levels and/or a reduced NR4A3 protein expression levels). In some aspects, the exhaustion or dysfunction in the modified immune cells is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to reference cells (e.g., CAR or TCR-expressing cells that have not been modified to express lower levels of NR4A3 gene and/or NR4A3 protein).

In some aspects, the modified cells disclosed herein can be used in combination with other therapeutic agents (e.g., anti-cancer agents and/or immunomodulating agents). Accordingly, in some aspects, a method of treating a tumor disclosed herein comprises administering the modified cells of the present disclosure in combination with one or more additional therapeutic agents to a subject. Such agents can include, for example, chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof. In some aspects, the modified cells disclosed herein (i.e., expressing reduced levels of NR4A3 gene and/or NR4A3 protein, alone or in combination with reduced level of the NR4A1 gene and/or NR4A1 protein and/or the NR4A2 gene and/or NR4A2 protein) can be used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, the modified cells of the present disclosure can be used in combination with one or more anti-cancer agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking T_{regs} or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits T_{regs}, such as T_{regs} in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUDA®; MK-3475), pidilizumab (CT-011), PDR001, MEDIO680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; R05541267), durvalumab (MEDI4736, IIFINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, an anti-cancer agent comprises an immune checkpoint activator (i.e., promotes signaling through the particular immune checkpoint pathway). In some aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof.

In some aspects, a modified cell disclosed herein is administered to the subject prior to or after the administration of the additional therapeutic agent. In some aspects, the modified cell is administered to the subject concurrently with the additional therapeutic agent. In some aspects, the modified cell and the additional therapeutic agent can be administered concurrently as a single composition in a pharmaceutically acceptable carrier. In some aspects, the modified cell and the additional therapeutic agent are administered concurrently as separate compositions. In some aspects, the additional therapeutic agent and the modified immune cells are administered sequentially.

### IV. Methods of Making Modified Immune Cells

The present disclosure provides methods of generating or preparing cells having a reduced level of NR4A3 gene and/or NR4A3 protein, comprising, e.g., modifying the cells with a gene editing tool, wherein the gene editing tool reduces the expression of the NR4A3 gene and/or NR4A3 protein. In some aspects, the reduced expression of the NR4A3 gene and/or NR4A3 protein reduces or inhibits exhaustion of the cells. In some aspects, the modified cells can be further modified to also have: (i) reduced level of NR4A1 gene and/or NR4A1 protein, (ii) reduced level of NR4A2 gene and/or NR4A2 protein, or (iii) both (i) and (ii). Accordingly, the present disclosure also provides methods of reducing or inhibiting exhaustion of cells expressing a chimeric antigen receptor (CAR) or a T cell receptor (TCR), comprising modifying the cells to reduce the expression level of NR4A3 gene and/or NR4A3 protein (alone or in combination with reduced level of the NR4A1 gene and/or NR4A1 protein and/or the NR4A2 gene and/or NR4A2 protein). In some aspects, the cells are immune cells. Also, the present disclosure provides methods of promoting a persistent effector function in immune cells, comprising modifying the cells to express reduced levels of NR4A3 gene and/or NR4A3 protein (alone or in combination with reduced level of the NR4A1 gene and/or NR4A1 protein and/or the NR4A2 gene and/or NR4A2 protein).

Gene editing, e.g., base editing, can be conducted using any editing tool known in the art. For example, in some aspects a modified cell (e.g., an immune cell) can be modified using techniques such as CRISPR/Cas, TALEN, Zinc finger nucleases (ZFN), meganucleases, restriction endonucleases, interference RNAs (RNAi), or antisense oligonucleotides. In some aspects, NR4A3 gene and/or expression can also be modified using shRNA, siRNA, or miRNA. All these techniques are discussed more in detail below. In some aspects, the method used for reducing the expression of NR4A3 gene and/or NR4A3 protein comprises using one or more gene editing tools (e.g., two, three, or more tools). In some aspects, the method used for reducing the expression of NR4A3 gene and/or NR4A3 protein comprises at least one method acting on NR4A3 DNA (e.g., CRISPR) or RNA (e.g., antisense oligonucleotides) and at least one method acting on NR4A3 protein (e.g., inhibition of binding to cell signaling partner or post-translational modifications).

In some aspects, cells (e.g., immune cells) modified as disclosed herein, e.g., by using a gene editing tool to reduce or abolish NR4A3 gene levels, can be further modified to express a CAR or a TCR. In some aspects, immune cells modified according to the gene editing methods disclosed herein and expressing a CAR or a TCR can have improved anticancer properties.

While the methods for reducing the expression of NR4A3 gene and/or NR4A3 protein, e.g., gene editing, are provided in the context of CAR- or TCR-expressing cells, those skilled in the art will recognize that the methods disclosed herein can be used for any cells, where reducing the expression of NR4A3 gene and/or NR4A3 protein is desired. For example, some aspects, the methods for reducing the expression of NR4A3 gene and/or NR4A3 protein disclosed herein can be applied to immune cells. In some aspects, the immune cell comprises a lymphocyte, neutrophil, monocyte, macrophage, dendritic cell, or combinations thereof. In some aspects, a lymphocyte comprises a T cell, tumor-infiltrating lymphocyte (TIL), lymphokine-activated killer cell, natural (NK) cell, or combinations thereof. In some aspects, a lymphocyte is a T cell, e.g., CD4⁺ T cell or a CD8⁺ T cell. In some aspects, a lymphocyte is a tumor infiltrating lymphocyte (TIL). In some aspects, a TIL is a CD8⁺ TIL. In some aspects, a TIL is a CD4⁺ TIL. Thus, the present disclosure provides cell compositions comprising modified cells (e.g., modified immune cells, wherein the parent cell is, for example, any cell disclosed above) prepared according to the methods for reducing the expression of NR4A3 gene and/or NR4A3 protein disclosed herein, wherein the modified cells exhibit reduced expression of NR4A3 gene and/or NR4A3 protein with respect to a reference cell (e.g., corresponding cell that has not been modified to express lower levels of NR4A3 gene and/or NR4A3 protein). In some aspects, these modified cells can be used to prepare a pharmaceutical composition.

In some aspects, modifying the cells comprises contacting the cells with a gene editing tool that is capable of reducing the expression levels of the NR4A3 gene and/or NR4A3 protein in the cell. In some aspects, the contacting of the gene editing tool (or any other tool capable of reducing expression of the NR4A3 gene and/or NR4A3 protein) with a cell to be modified can occur in vivo, in vitro, ex vivo, or combinations thereof. In some aspects, the contacting occurs in vivo (e.g., gene therapy). In some aspects, the contacting occurs in vitro. In some aspects, the contacting occurs ex vivo. In some aspects, the cell is an autologous cell. In some aspects, the cell is a heterologous cell. In some aspects, the contacting of the gene editing tool (or any other tool capable of reducing expression of the NR4A3 gene and/or NR4A3 protein) reduces the expression level of the NR4A3 gene and/or NR4A3 protein in the cells by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the level of NR4A3 gene and/or NR4A3 protein in a reference cell (e.g., corresponding cell that has not been modified to express lower levels of NR4A3 gene and/or NR4A3 protein).

In some aspects, contacting a cell with a gene editing tool comprises different routes of delivery. Generally, for the gene editing tools disclosed herein to reduce the expression of NR4A3 gene and/or NR4A3 protein in a cell, the gene editing tool must be able to enter the cell and bind to the gene of interest. In some aspects, any delivery vehicle known in the art for delivering molecules of interest to a cell can be used. See, e.g., U.S. Pat. No. 10,047,355 B2, which is herein incorporated by reference in its entirety. Additional disclosure relating to vectors that can be used are provided elsewhere in the present disclosure.

In some aspects, a gene editing tool can remove the entire gene encoding a NR4A3 protein. In some aspects, a gene editing tool removes a portion (e.g., one or more exons) of the genome encoding a NR4A3 protein. In some aspects, a gene editing tool, e.g., a base editor, modifies a specific nucleotide base without generating an indel. As used herein, the term “indel” refers to the insertion or deletion of a nucleotide base within a nucleic acid that can lead to frame shift mutations within a coding region of a gene. Non-limiting examples of base editors are disclosed in U.S. Publication No. 2017/0121693, published May 4, 2017, which is incorporated herein by reference in its entirety.

In some aspects, a method of preparing a modified immune cell further comprises modifying the cell to express a CAR or a TCR. In some aspects, modifying the cell to express a CAR or TCR comprises contacting the cell with a nucleic acid sequence encoding the CAR. In some aspects, the nucleic acid sequence encoding the CAR is expressed from a vector (e.g., expression vector).

In some aspects, a gene editing tool disclosed herein is expressed from a vector comprising a nucleic acid sequence encoding the gene editing tool. In some aspects, the nucleic acid sequence encoding the gene editing tool and the nucleic acid sequence encoding the CAR or TCR are on separate vectors. In some aspects, the nucleic acid sequence encoding the gene editing tool and the nucleic acid sequence encoding the CAR or TCR are on the same vector.

### V.A. Gene Editing Tools

One or more gene editing tools can be used to modify the cells of the present disclosure. Non-limiting examples of the gene editing tools are disclosed below:

### V.A.1. CRISPR/Cas System

In some aspects, the gene editing tool that can be used in the present disclosure comprises a CRISPR/Cas system. Such systems can employ, for example, a nucleic acid molecule encoding a Cas9 nuclease, which in some instances, is codon-optimized for the desired cell type in which it is to be expressed (e.g., T cells, e.g., CAR-expressing T cells). As further described herein, in some aspects, such a system can comprise a Cas9 nuclease protein.

CRISPR/Cas systems use Cas nucleases, e.g., Cas9 nucleases, that are targeted to a genomic site by complexing with a guide RNA (e.g., synthetic guide RNA) (gRNA) that hybridizes to a target DNA sequence immediately preceding an NGG motif recognized by the Cas nuclease, e.g., Cas9. This results in a double-strand break three nucleotides upstream of the NGG motif. A unique capability of the CRISPR/Cas9 system is the ability to simultaneously target multiple distinct genomic loci by co-expressing a single Cas9 protein with two or more gRNAs (e.g., at least one, two, three, four, five, six, seven, eight, nine or ten gRNAs). Such systems can also employ a guide RNA that comprises two separate molecules. In some aspects, the two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA is complementary to and hybridizes with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence (e.g., NR4A3 mRNA), and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used (e.g., humans).

Naturally-occurring genes encoding the three elements (Cas9, tracrRNA and crRNA) are typically organized in operon(s). Naturally-occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, a CRISPR system used herein can further employ a fused crRNA-tracrRNA construct (i.e., a single transcript) that functions with the codon-optimized Cas9. This single RNA is often referred to as a guide RNA or gRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid.

The gRNA expression cassette and the Cas9 expression cassette are then introduced into the cell. See, for example, Mali P et al., (2013) Science 2013 Feb. 15; 339(6121):823-6; Jinek M et al., Science 2012 Aug. 17; 337(6096):816-21; Hwang W Y et al., Nat Biotechnol 2013 March; 31(3):227-9; Jiang W et al., Nat Biotechnol 2013 March; 31(3):233-9; and Cong L et al., Science 2013 Feb. 15; 339(6121):819-23, each of which is herein incorporated by reference in its entirety. See also, for example, WO/2013/176772 A1, WO/2014/065596 A1, WO/2014/089290 A1, WO/2014/093622 A2, WO/2014/099750 A2, and WO/2013142578 A1, each of which is herein incorporated by reference in its entirety.

In some aspects, the Cas9 nuclease can be provided in the form of a protein. For instance, in some aspects, a cell useful for the present disclosure (e.g., CAR or TCR expressing immune cell) can be modified (e.g., to have reduced level a NR4A gene and/or NR4A protein) by introducing a Cas9 nuclease protein and a nucleic acid molecule comprising a gRNA. In some aspects, the Cas9 nuclease protein and the nucleic acid molecule comprising a gRNA can be introduced into the cell sequentially. In some aspects, the Cas9 nuclease protein and the nucleic acid molecule comprising a gRNA can be introduced into the cell concurrently. For instance, in some aspects, the concurrent administration comprises introducing the Cas9 nuclease protein and the nucleic acid molecule comprising a gRNA at the same time but as separate compositions. In some aspects, the Cas9 protein can be provided in the form of a complex with the nucleic acid molecule comprising a gRNA (i.e., as a single composition).

In some aspects, the Cas9 nuclease can be provided in the form of a nucleic acid encoding the protein. Accordingly, in some aspects, a cell useful for the present disclosure (e.g., CAR or TCR expressing immune cell) can be modified (e.g., to have reduced level of a NR4A gene and/or NR4A protein) by introducing a first nucleic acid molecule encoding a Cas9 nuclease protein and a second nucleic acid molecule comprising a gRNA. In some aspects, the first and second nucleic acid molecules can be introduced the cell sequentially. In some aspects, the first and second nucleic acid molecules can be introduced into the cell concurrently. For instance, in some aspects, the first and second nucleic acid molecules can be introduced into the cell at the same time but as separate compositions. In some aspects, the first and second nucleic acid molecules can be part of a single polynucleotide, and the cell is modified to comprise the single polynucleotide.

The nucleic acid encoding the Cas9 nuclease can be RNA (e.g., messenger RNA (mRNA)) or DNA. In some aspects, the gRNA can be provided in the form of RNA. In some aspects, the gRNA can be provided in the form of DNA encoding the RNA. In some aspects, the gRNA can be provided in the form of separate crRNA and tracrRNA molecules, or separate DNA molecules encoding the crRNA and tracrRNA, respectively.

In some aspects, the gRNA comprises a third nucleic acid sequence encoding a Clustered Regularly Interspaced Short Palindromic Repeats (CRISPR) RNA (crRNA) and a trans-activating CRISPR RNA (tracrRNA). In some aspects, the Cas protein is a type I Cas protein. In some aspects, the Cas protein is a type II Cas protein. In some aspects, the type II Cas protein is Cas9. In some aspects, the type II Cas, e.g., Cas9, is a human codon-optimized Cas.

In some aspects, the Cas protein is a “nickase” that can create single strand breaks (i.e., “nicks”) within the target nucleic acid sequence without cutting both strands of double stranded DNA (dsDNA). Cas9, for example, comprises two nuclease domains—a RuvC-like nuclease domain and an HNH-like nuclease domain—which are responsible for cleavage of opposite DNA strands. Mutation in either of these domains can create a nickase. Examples of mutations creating nickases can be found, for example, WO/2013/176772 A1 and WO/2013/142578 A1, each of which is herein incorporated by reference.

In some aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the expression of NR4A3 gene, and the NR4A3 protein encoded thereof, is reduced by contacting the cell with a CRISPR (e.g., CRISPR-Cas9 system) that is, e.g., specific to the NR4A3 gene. In some aspects, the CRISPR is specific to the NR4A1 gene. Accordingly, in some aspects, after the contacting with the CRISPR, the cell (e.g., CAR or TCR expressing immune cell) has: (i) a reduced level of the NR4A1 gene and/or protein, (ii) endogenous level of the NR4A2 gene and/or protein, and (iii) endogenous level of the NR4A3 gene and/or protein. In some aspects, the CRISPR is specific for the NR4A2 gene. Accordingly, in some aspects, after the contacting with the CRISPR, the cell (e.g., CAR or TCR expressing immune cell) has: (i) endogenous level of the NR4A1 gene and/or protein, (ii) reduced level of the NR4A2 gene and/or protein, and (iii) endogenous level of the NR4A3 gene and/or protein. In some aspects, the CRISPR is specific for the NR4A3 gene. Accordingly, in some aspects, after the contacting with the CRISPR, the cell (e.g., CAR or TCR expressing immune cell) has: (i) endogenous level of the NR4A1 gene and/or protein, (ii) endogenous level of the NR4A2 gene and/or protein, and (iii) reduced level of the NR4A3 gene and/or protein.

As described herein, in some aspects, the CRISPR targets multiple NR4A genes. For instance, in some aspects, the CRISPR is capable of targeting both the NR4A1 gene and the NR4A2 gene. Accordingly, in some aspects, after the contacting with the CRISPR, the cell (e.g., CAR or TCR expressing immune cell) has: (i) reduced level of the NR4A1 gene and/or protein, (ii) reduced level of the NR4A2 gene and/or protein, and (iii) endogenous level of the NR4A3 gene and/or protein. In some aspects, the CRISPR is capable of targeting both the NR4A1 gene and the NR4A3 gene. Accordingly, in some aspects, after the contacting with the CRISPR, the cell (e.g., CAR or TCR expressing immune cell) has: (i) reduced level of the NR4A1 gene and/or protein, (ii) endogenous level of the NR4A2 gene and/or protein, and (iii) reduced level of the NR4A3 gene and/or protein. In some aspects, the CRISPR is capable of targeting both the NR4A2 gene and/or the NR4A3 gene. In some aspects, after the contacting with the CRISPR, the cell (e.g., CAR or TCR expressing immune cell) has: (i) endogenous level of the NR4A1 gene and/or protein, (ii) reduced level of the NR4A2 gene and/or protein, and (iii) reduced level of the NR4A3 gene and/or protein. In some aspects, the CRISPR is capable of targeting the NR4A1 gene, the NR4A2 gene, and the NR4A3 gene. Accordingly, in some aspects, after the contacting with the CRISPR, the cell (e.g., CAR or TCR expressing immune cell) has: (i) reduced level of the NR4A1 gene and/or protein, (ii) reduced level of the NR4A2 gene and/or protein, and (iii) reduced level of the NR4A3 gene and/or protein.

In some aspects, gene editing using CRISPR reduces NR4A3 gene levels at least about 10%, at least about 15%, at least about 20%, at least about 25%, at least about 30%, at least about 35%, at least about 40%, at least about 45%, at least about 50%, at least about 55%, at least about 60%, at least about 65%, at least 70%, at least about 75%, at least about 80%, at least about 85%, at least about 90%, at least about 95%, or about 100% with respect to NR4A3 gene levels observed in a reference cell (e.g., a corresponding cell that has not been subjected to gene editing using CRISPR). In some aspects, NR4A3 gene levels can be measured using any technique known in the art, e.g., by digital droplet PCR.

In some aspects, a nucleic acid encoding a gRNA and/or a Cas9 disclosed herein is an RNA or a DNA. In other aspect, the RNA or DNA encoding a gRNA and/or a Cas9 disclosed herein is a synthetic RNA or a synthetic DNA, respectively. In some aspects, the synthetic RNA or DNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine or pseudouridine). In some aspects, the polynucleotide (e.g., a synthetic RNA or a synthetic DNA) comprises only natural nucleobases, i.e., A, C, T and U in the case of a synthetic DNA, or A, C, T, and U in the case of a synthetic RNA or synthetic DNA.

In general, the CRISPR gene editing methods disclosed herein comprise contacting a cell, e.g., an immune cell, in vivo, in vitro, or ex vivo with
(i) a Cas9 or a nucleic acid encoding the Cas9; and,
(ii) at least one NR4A3 gene guide RNA (gRNA) or a nucleic acid encoding the gRNA, wherein the gRNA targets a sequence in the NR4A3 gene (e.g., an intron and/or exon sequence), wherein contacting the cell with the Cas9 and the at least one gRNA results in a reduction of the expression of the NR4A3 gene and/or NR4A3 protein.

In some aspects, a gRNA that can be used to reduce the level of a NR4A3 gene of a cell (e.g., immune cell) comprises any of the gRNAs provided Tables B and C. For instance, in some aspects, a gRNA that can be used to target the NR4A3 gene comprises, consists of, or consists essentially of any one or more of the sequences set forth in SEQ ID NOs: 30, 52-57, 58, 61, 65, 67, 68, 70, 71, 75, 76, 82, 83, 86, 94, and 96. In some aspects, a gRNA that can be used to target the NR4A3 gene comprises, consists of, or consists essentially of the sequence set forth in SEQ ID NO: 30. In some aspects, a gRNA that can be used to target the NR4A3 gene comprises the sequence set forth in SEQ ID NO: 30. In some aspects, a gRNA that can be used to target the NR4A3 gene consists of the sequence set forth in SEQ ID NO: 30. In some aspects, a gRNA that can be used to target the NR4A3 gene consists essentially of the sequence set forth in SEQ ID NO: 30. In some aspects, a gRNA that can be used to target the NR4A3 gene comprises, consists of, or consists essentially of the sequence set forth in SEQ ID NO: 52. In some aspects, a gRNA that can be used to target the NR4A3 gene comprises the sequence set forth in SEQ ID NO: 52. In some aspects, a gRNA that can be used to target the NR4A3 gene consists of the sequence set forth in SEQ ID NO: 52. In some aspects, a gRNA that can be used to target the NR4A3 gene consists essentially of the sequence set forth in SEQ ID NO: 52. In some aspects, a gRNA that can be used to target the NR4A3 gene comprises, consists of, or consists essentially of the sequence set forth in SEQ ID NO: 53. In some aspects, a gRNA that can be used to target the NR4A3 gene comprises the sequence set forth in SEQ ID NO: 53. In some aspects, a gRNA that can be used to target the NR4A3 gene consists of the sequence set forth in SEQ ID NO: 53. In some aspects, a gRNA that can be used to target the NR4A3 gene consists essentially of the sequence set forth in SEQ ID NO: 53. In some aspects, a gRNA that can be used to target the NR4A3 gene comprises, consists of, or consists essentially of the sequence set forth in SEQ ID NO: 54. In some aspects, a gRNA that can be used to target the NR4A3 gene comprises the sequence set forth in SEQ ID NO: 54. In some aspects, a gRNA that can be used to target the NR4A3 gene consists of the sequence set forth in SEQ ID NO: 54. In some aspects, a gRNA that can be used to target the NR4A3 gene consists essentially of the sequence set forth in SEQ ID NO: 54. In some aspects, a gRNA that can be used to target the NR4A3 gene comprises, consists of, or consists essentially of the sequence set forth in SEQ ID NO: 55. In some aspects, a gRNA that can be used to target the NR4A3 gene comprises the sequence set forth in SEQ ID NO: 55. In some aspects, a gRNA that can be used to target the NR4A3 gene consists of the sequence set forth in SEQ ID NO: 55. In some aspects, a gRNA that can be used to target the NR4A3 gene consists essentially of the sequence set forth in SEQ ID NO: 55. In some aspects, a gRNA that can be used to target the NR4A3 gene comprises, consists of, or consists essentially of the sequence set forth in SEQ ID NO: 53. In some aspects, a gRNA that can be used to target the NR4A3 gene comprises the sequence set forth in SEQ ID NO: 56. In some aspects, a gRNA that can be used to target the NR4A3 gene consists of the sequence set forth in SEQ ID NO: 56. In some aspects, a gRNA that can be used to target the NR4A3 gene consists essentially of the sequence set forth in SEQ ID NO: 56. In some aspects, a gRNA that can be used to target the NR4A3 gene comprises, consists of, or consists essentially of the sequence set forth in SEQ ID NO: 57. In some aspects, a gRNA that can be used to target the NR4A3 gene comprises the sequence set forth in SEQ ID NO: 57. In some aspects, a gRNA that can be used to target the NR4A3 gene consists of the sequence set forth in SEQ ID NO: 57. In some aspects, a gRNA that can be used to target the NR4A3 gene consists essentially of the sequence set forth in SEQ ID NO: 57. In some aspects, a gRNA that can be used to target the NR4A3 gene comprises, consists of, or consists essentially of the sequence set forth in SEQ ID NO: 58. In some aspects, a gRNA that can be used to target the NR4A3 gene comprises the sequence set forth in SEQ ID NO: 58. In some aspects, a gRNA that can be used to target the NR4A3 gene consists of the sequence set forth in SEQ ID NO: 58. In some aspects, a gRNA that can be used to target the NR4A3 gene consists essentially of the sequence set forth in SEQ ID NO: 58. In some aspects, a gRNA that can be used to target the NR4A3 gene comprises, consists of, or consists essentially of the sequence set forth in SEQ ID NO: 59. In some aspects, a gRNA that can be used to target the NR4A3 gene comprises the sequence set forth in SEQ ID NO: 59. In some aspects, a gRNA that can be used to target the NR4A3 gene consists of the sequence set forth in SEQ ID NO: 59. In some aspects, a gRNA that can be used to target the NR4A3 gene consists essentially of the sequence set forth in SEQ ID NO: 59. In some aspects, a gRNA that can be used to target the NR4A3 gene comprises, consists of, or consists essentially of the sequence set forth in SEQ ID NO: 60. In some aspects, a gRNA that can be used to target the NR4A3 gene comprises the sequence set forth in SEQ ID NO: 60. In some aspects, a gRNA that can be used to target the NR4A3 gene consists of the sequence set forth in SEQ ID NO: 60. In some aspects, a gRNA that can be used to target the NR4A3 gene consists essentially of the sequence set forth in SEQ ID NO: 60. In some aspects, a gRNA that can be used to target the NR4A3 gene comprises, consists of, or consists essentially of the sequence set forth in SEQ ID NO: 61. In some aspects, a gRNA that can be used to target the NR4A3 gene comprises the sequence set forth in SEQ ID NO: 61. In some aspects, a gRNA that can be used to target the NR4A3 gene consists of the sequence set forth in SEQ ID NO: 61. In some aspects, a gRNA that can be used to target the NR4A3 gene consists essentially of the sequence set forth in SEQ ID NO: 61. In some aspects, a gRNA that can be used to target the NR4A3 gene comprises, consists of, or consists essentially of the sequence set forth in SEQ ID NO: 62. In some aspects, a gRNA that can be used to target the NR4A3 gene comprises the sequence set forth in SEQ ID NO: 62. In some aspects, a gRNA that can be used to target the NR4A3 gene consists of the sequence set forth in SEQ ID NO: 62. In some aspects, a gRNA that can be used to target the NR4A3 gene consists essentially of the sequence set forth in SEQ ID NO: 62. In some aspects, a gRNA that can be used to target the NR4A3 gene comprises, consists of, or consists essentially of the sequence set forth in SEQ ID NO: 63. In some aspects, a gRNA that can be used to target the NR4A3 gene comprises the sequence set forth in SEQ ID NO: 63. In some aspects, a gRNA that can be used to target the NR4A3 gene consists of the sequence set forth in SEQ ID NO: 63. In some aspects, a gRNA that can be used to target the NR4A3 gene consists essentially of the sequence set forth in SEQ ID NO: 63. In some aspects, a gRNA that can be used to target the NR4A3 gene comprises, consists of, or consists essentially of the sequence set forth in SEQ ID NO: 64. In some aspects, a gRNA that can be used to target the NR4A3 gene comprises the sequence set forth in SEQ ID NO: 64. In some aspects, a gRNA that can be used to target the NR4A3 gene consists of the sequence set forth in SEQ ID NO: 64. In some aspects, a gRNA that can be used to target the NR4A3 gene consists essentially of the sequence set forth in SEQ ID NO: 64. In some aspects, a gRNA that can be used to target the NR4A3 gene comprises, consists of, or consists essentially of the sequence set forth in SEQ ID NO: 65. In some aspects, a gRNA that can be used to target the NR4A3 gene comprises the sequence set forth in SEQ ID NO: 65. In some aspects, a gRNA that can be used to target the NR4A3 gene consists of the sequence set forth in SEQ ID NO: 65. In some aspects, a gRNA that can be used to target the NR4A3 gene consists essentially of the sequence set forth in SEQ ID NO: 65. In some aspects, a gRNA that can be used to target the NR4A3 gene comprises, consists of, or consists essentially of the sequence set forth in SEQ ID NO: 66. In some aspects, a gRNA that can be used to target the NR4A3 gene comprises the sequence set forth in SEQ ID NO: 66. In some aspects, a gRNA that can be used to target the NR4A3 gene consists of the sequence set forth in SEQ ID NO: 66. In some aspects, a gRNA that can be used to target the NR4A3 gene consists essentially of the sequence set forth in SEQ ID NO: 66. In some aspects, a gRNA that can be used to target the NR4A3 gene comprises, consists of, or consists essentially of the sequence set forth in SEQ ID NO: 67. In some aspects, a gRNA that can be used to target the NR4A3 gene comprises the sequence set forth in SEQ ID NO: 67. In some aspects, a gRNA that can be used to target the NR4A3 gene consists of the sequence set forth in SEQ ID NO: 67. In some aspects, a gRNA that can be used to target the NR4A3 gene consists essentially of the sequence set forth in SEQ ID NO: 67. In some aspects, a gRNA that can be used to target the NR4A3 gene comprises, consists of, or consists essentially of the sequence set forth in SEQ ID NO: 68. In some aspects, a gRNA that can be used to target the NR4A3 gene comprises the sequence set forth in SEQ ID NO: 68. In some aspects, a gRNA that can be used to target the NR4A3 gene consists of the sequence set forth in SEQ ID NO: 68. In some aspects, a gRNA that can be used to target the NR4A3 gene consists essentially of the sequence set forth in SEQ ID NO: 68. In some aspects, a gRNA that can be used to target the NR4A3 gene comprises, consists of, or consists essentially of the sequence set forth in SEQ ID NO: 69. In some aspects, a gRNA that can be used to target the NR4A3 gene comprises the sequence set forth in SEQ ID NO: 69. In some aspects, a gRNA that can be used to target the NR4A3 gene consists of the sequence set forth in SEQ ID NO: 69. In some aspects, a gRNA that can be used to target the NR4A3 gene consists essentially of the sequence set forth in SEQ ID NO: 69. In some aspects, a gRNA that can be used to target the NR4A3 gene comprises, consists of, or consists essentially of the sequence set forth in SEQ ID NO: 70. In some aspects, a gRNA that can be used to target the NR4A3 gene comprises the sequence set forth in SEQ ID NO: 70. In some aspects, a gRNA that can be used to target the NR4A3 gene consists of the sequence set forth in SEQ ID NO: 70. In some aspects, a gRNA that can be used to target the NR4A3 gene consists essentially of the sequence set forth in SEQ ID NO: 70. In some aspects, a gRNA that can be used to target the NR4A3 gene comprises, consists of, or consists essentially of the sequence set forth in SEQ ID NO: 71. In some aspects, a gRNA that can be used to target the NR4A3 gene comprises the sequence set forth in SEQ ID NO: 71. In some aspects, a gRNA that can be used to target the NR4A3 gene consists of the sequence set forth in SEQ ID NO: 71. In some aspects, a gRNA that can be used to target the NR4A3 gene consists essentially of the sequence set forth in SEQ ID NO: 71. In some aspects, a gRNA that can be used to target the NR4A3 gene comprises, consists of, or consists essentially of the sequence set forth in SEQ ID NO: 72. In some aspects, a gRNA that can be used to target the NR4A3 gene comprises the sequence set forth in SEQ ID NO: 72. In some aspects, a gRNA that can be used to target the NR4A3 gene consists of the sequence set forth in SEQ ID NO: 72. In some aspects, a gRNA that can be used to target the NR4A3 gene consists essentially of the sequence set forth in SEQ ID NO: 72. In some aspects, a gRNA that can be used to target the NR4A3 gene comprises, consists of, or consists essentially of the sequence set forth in SEQ ID NO: 73. In some aspects, a gRNA that can be used to target the NR4A3 gene comprises the sequence set forth in SEQ ID NO: 73. In some aspects, a gRNA that can be used to target the NR4A3 gene consists of the sequence set forth in SEQ ID NO: 73. In some aspects, a gRNA that can be used to target the NR4A3 gene consists essentially of the sequence set forth in SEQ ID NO: 73. In some aspects, a gRNA that can be used to target the NR4A3 gene comprises, consists of, or consists essentially of the sequence set forth in SEQ ID NO: 74. In some aspects, a gRNA that can be used to target the NR4A3 gene comprises the sequence set forth in SEQ ID NO: 74. In some aspects, a gRNA that can be used to target the NR4A3 gene consists of the sequence set forth in SEQ ID NO: 74. In some aspects, a gRNA that can be used to target the NR4A3 gene consists essentially of the sequence set forth in SEQ ID NO: 74. In some aspects, a gRNA that can be used to target the NR4A3 gene comprises, consists of, or consists essentially of the sequence set forth in SEQ ID NO: 75. In some aspects, a gRNA that can be used to target the NR4A3 gene comprises the sequence set forth in SEQ ID NO: 75. In some aspects, a gRNA that can be used to target the NR4A3 gene consists of the sequence set forth in SEQ ID NO: 75. In some aspects, a gRNA that can be used to target the NR4A3 gene consists essentially of the sequence set forth in SEQ ID NO: 75. In some aspects, a gRNA that can be used to target the NR4A3 gene comprises, consists of, or consists essentially of the sequence set forth in SEQ ID NO: 76. In some aspects, a gRNA that can be used to target the NR4A3 gene comprises the sequence set forth in SEQ ID NO: 76. In some aspects, a gRNA that can be used to target the NR4A3 gene consists of the sequence set forth in SEQ ID NO: 76. In some aspects, a gRNA that can be used to target the NR4A3 gene consists essentially of the sequence set forth in SEQ ID NO: 76. In some aspects, a gRNA that can be used to target the NR4A3 gene comprises, consists of, or consists essentially of the sequence set forth in SEQ ID NO: 77. In some aspects, a gRNA that can be used to target the NR4A3 gene comprises the sequence set forth in SEQ ID NO: 77. In some aspects, a gRNA that can be used to target the NR4A3 gene consists of the sequence set forth in SEQ ID NO: 77. In some aspects, a gRNA that can be used to target the NR4A3 gene consists essentially of the sequence set forth in SEQ ID NO: 77. In some aspects, a gRNA that can be used to target the NR4A3 gene comprises, consists of, or consists essentially of the sequence set forth in SEQ ID NO: 78. In some aspects, a gRNA that can be used to target the NR4A3 gene comprises the sequence set forth in SEQ ID NO: 78. In some aspects, a gRNA that can be used to target the NR4A3 gene consists of the sequence set forth in SEQ ID NO: 78. In some aspects, a gRNA that can be used to target the NR4A3 gene consists essentially of the sequence set forth in SEQ ID NO: 78. In some aspects, a gRNA that can be used to target the NR4A3 gene comprises, consists of, or consists essentially of the sequence set forth in SEQ ID NO: 79. In some aspects, a gRNA that can be used to target the NR4A3 gene comprises the sequence set forth in SEQ ID NO: 79. In some aspects, a gRNA that can be used to target the NR4A3 gene consists of the sequence set forth in SEQ ID NO: 79. In some aspects, a gRNA that can be used to target the NR4A3 gene consists essentially of the sequence set forth in SEQ ID NO: 79. In some aspects, a gRNA that can be used to target the NR4A3 gene comprises, consists of, or consists essentially of the sequence set forth in SEQ ID NO: 80. In some aspects, a gRNA that can be used to target the NR4A3 gene comprises the sequence set forth in SEQ ID NO: 80. In some aspects, a gRNA that can be used to target the NR4A3 gene consists of the sequence set forth in SEQ ID NO: 80. In some aspects, a gRNA that can be used to target the NR4A3 gene consists essentially of the sequence set forth in SEQ ID NO: 80. In some aspects, a gRNA that can be used to target the NR4A3 gene comprises, consists of, or consists essentially of the sequence set forth in SEQ ID NO: 81. In some aspects, a gRNA that can be used to target the NR4A3 gene comprises the sequence set forth in SEQ ID NO: 81. In some aspects, a gRNA that can be used to target the NR4A3 gene consists of the sequence set forth in SEQ ID NO: 81. In some aspects, a gRNA that can be used to target the NR4A3 gene consists essentially of the sequence set forth in SEQ ID NO: 81. In some aspects, a gRNA that can be used to target the NR4A3 gene comprises, consists of, or consists essentially of the sequence set forth in SEQ ID NO: 82. In some aspects, a gRNA that can be used to target the NR4A3 gene comprises the sequence set forth in SEQ ID NO: 82. In some aspects, a gRNA that can be used to target the NR4A3 gene consists of the sequence set forth in SEQ ID NO: 82. In some aspects, a gRNA that can be used to target the NR4A3 gene consists essentially of the sequence set forth in SEQ ID NO: 82. In some aspects, a gRNA that can be used to target the NR4A3 gene comprises, consists of, or consists essentially of the sequence set forth in SEQ ID NO: 83. In some aspects, a gRNA that can be used to target the NR4A3 gene comprises the sequence set forth in SEQ ID NO: 83. In some aspects, a gRNA that can be used to target the NR4A3 gene consists of the sequence set forth in SEQ ID NO: 83. In some aspects, a gRNA that can be used to target the NR4A3 gene consists essentially of the sequence set forth in SEQ ID NO: 83. In some aspects, a gRNA that can be used to target the NR4A3 gene comprises, consists of, or consists essentially of the sequence set forth in SEQ ID NO: 84. In some aspects, a gRNA that can be used to target the NR4A3 gene comprises the sequence set forth in SEQ ID NO: 84. In some aspects, a gRNA that can be used to target the NR4A3 gene consists of the sequence set forth in SEQ ID NO: 84. In some aspects, a gRNA that can be used to target the NR4A3 gene consists essentially of the sequence set forth in SEQ ID NO: 84. In some aspects, a gRNA that can be used to target the NR4A3 gene comprises, consists of, or consists essentially of the sequence set forth in SEQ ID NO: 85. In some aspects, a gRNA that can be used to target the NR4A3 gene comprises the sequence set forth in SEQ ID NO: 85. In some aspects, a gRNA that can be used to target the NR4A3 gene consists of the sequence set forth in SEQ ID NO: 85. In some aspects, a gRNA that can be used to target the NR4A3 gene consists essentially of the sequence set forth in SEQ ID NO: 85. In some aspects, a gRNA that can be used to target the NR4A3 gene comprises, consists of, or consists essentially of the sequence set forth in SEQ ID NO: 86. In some aspects, a gRNA that can be used to target the NR4A3 gene comprises the sequence set forth in SEQ ID NO: 86. In some aspects, a gRNA that can be used to target the NR4A3 gene consists of the sequence set forth in SEQ ID NO: 86. In some aspects, a gRNA that can be used to target the NR4A3 gene consists essentially of the sequence set forth in SEQ ID NO: 86. In some aspects, a gRNA that can be used to target the NR4A3 gene comprises, consists of, or consists essentially of the sequence set forth in SEQ ID NO: 87. In some aspects, a gRNA that can be used to target the NR4A3 gene comprises the sequence set forth in SEQ ID NO: 87. In some aspects, a gRNA that can be used to target the NR4A3 gene consists of the sequence set forth in SEQ ID NO: 87. In some aspects, a gRNA that can be used to target the NR4A3 gene consists essentially of the sequence set forth in SEQ ID NO: 87. In some aspects, a gRNA that can be used to target the NR4A3 gene comprises, consists of, or consists essentially of the sequence set forth in SEQ ID NO: 88. In some aspects, a gRNA that can be used to target the NR4A3 gene comprises the sequence set forth in SEQ ID NO: 88. In some aspects, a gRNA that can be used to target the NR4A3 gene consists of the sequence set forth in SEQ ID NO: 88. In some aspects, a gRNA that can be used to target the NR4A3 gene consists essentially of the sequence set forth in SEQ ID NO: 88. In some aspects, a gRNA that can be used to target the NR4A3 gene comprises, consists of, or consists essentially of the sequence set forth in SEQ ID NO: 89. In some aspects, a gRNA that can be used to target the NR4A3 gene comprises the sequence set forth in SEQ ID NO: 89. In some aspects, a gRNA that can be used to target the NR4A3 gene consists of the sequence set forth in SEQ ID NO: 89. In some aspects, a gRNA that can be used to target the NR4A3 gene consists essentially of the sequence set forth in SEQ ID NO: 89. In some aspects, a gRNA that can be used to target the NR4A3 gene comprises, consists of, or consists essentially of the sequence set forth in SEQ ID NO: 90. In some aspects, a gRNA that can be used to target the NR4A3 gene comprises the sequence set forth in SEQ ID NO: 90. In some aspects, a gRNA that can be used to target the NR4A3 gene consists of the sequence set forth in SEQ ID NO: 90. In some aspects, a gRNA that can be used to target the NR4A3 gene consists essentially of the sequence set forth in SEQ ID NO: 90. In some aspects, a gRNA that can be used to target the NR4A3 gene comprises, consists of, or consists essentially of the sequence set forth in SEQ ID NO: 91. In some aspects, a gRNA that can be used to target the NR4A3 gene comprises the sequence set forth in SEQ ID NO: 91. In some aspects, a gRNA that can be used to target the NR4A3 gene consists of the sequence set forth in SEQ ID NO: 91. In some aspects, a gRNA that can be used to target the NR4A3 gene consists essentially of the sequence set forth in SEQ ID NO: 91. In some aspects, a gRNA that can be used to target the NR4A3 gene comprises, consists of, or consists essentially of the sequence set forth in SEQ ID NO: 92. In some aspects, a gRNA that can be used to target the NR4A3 gene comprises the sequence set forth in SEQ ID NO: 92. In some aspects, a gRNA that can be used to target the NR4A3 gene consists of the sequence set forth in SEQ ID NO: 92. In some aspects, a gRNA that can be used to target the NR4A3 gene consists essentially of the sequence set forth in SEQ ID NO: 92. In some aspects, a gRNA that can be used to target the NR4A3 gene comprises, consists of, or consists essentially of the sequence set forth in SEQ ID NO: 93. In some aspects, a gRNA that can be used to target the NR4A3 gene comprises the sequence set forth in SEQ ID NO: 93. In some aspects, a gRNA that can be used to target the NR4A3 gene consists of the sequence set forth in SEQ ID NO: 93. In some aspects, a gRNA that can be used to target the NR4A3 gene consists essentially of the sequence set forth in SEQ ID NO: 93. In some aspects, a gRNA that can be used to target the NR4A3 gene comprises, consists of, or consists essentially of the sequence set forth in SEQ ID NO: 94. In some aspects, a gRNA that can be used to target the NR4A3 gene comprises the sequence set forth in SEQ ID NO: 94. In some aspects, a gRNA that can be used to target the NR4A3 gene consists of the sequence set forth in SEQ ID NO: 94. In some aspects, a gRNA that can be used to target the NR4A3 gene consists essentially of the sequence set forth in SEQ ID NO: 94. In some aspects, a gRNA that can be used to target the NR4A3 gene comprises, consists of, or consists essentially of the sequence set forth in SEQ ID NO: 95. In some aspects, a gRNA that can be used to target the NR4A3 gene comprises the sequence set forth in SEQ ID NO: 95. In some aspects, a gRNA that can be used to target the NR4A3 gene consists of the sequence set forth in SEQ ID NO: 95. In some aspects, a gRNA that can be used to target the NR4A3 gene consists essentially of the sequence set forth in SEQ ID NO: 95. In some aspects, a gRNA that can be used to target the NR4A3 gene comprises, consists of, or consists essentially of the sequence set forth in SEQ ID NO: 96. In some aspects, a gRNA that can be used to target the NR4A3 gene comprises the sequence set forth in SEQ ID NO: 96. In some aspects, a gRNA that can be used to target the NR4A3 gene consists of the sequence set forth in SEQ ID NO: 96. In some aspects, a gRNA that can be used to target the NR4A3 gene consists essentially of the sequence set forth in SEQ ID NO: 96. In some aspects, a gRNA that can be used to target the NR4A3 gene comprises, consists of, or consists essentially of the sequence set forth in SEQ ID NO: 97. In some aspects, a gRNA that can be used to target the NR4A3 gene comprises the sequence set forth in SEQ ID NO: 97. In some aspects, a gRNA that can be used to target the NR4A3 gene consists of the sequence set forth in SEQ ID NO: 97. In some aspects, a gRNA that can be used to target the NR4A3 gene consists essentially of the sequence set forth in SEQ ID NO: 97. In some aspects, a gRNA that can be used to target the NR4A3 gene comprises, consists of, or consists essentially of the sequence set forth in SEQ ID NO: 98. In some aspects, a gRNA that can be used to target the NR4A3 gene comprises the sequence set forth in SEQ ID NO: 98. In some aspects, a gRNA that can be used to target the NR4A3 gene consists of the sequence set forth in SEQ ID NO: 98. In some aspects, a gRNA that can be used to target the NR4A3 gene consists essentially of the sequence set forth in SEQ ID NO: 98. In some aspects, a gRNA that can be used to target the NR4A3 gene comprises, consists of, or consists essentially of the sequence set forth in SEQ ID NO: 99. In some aspects, a gRNA that can be used to target the NR4A3 gene comprises the sequence set forth in SEQ ID NO: 99. In some aspects, a gRNA that can be used to target the NR4A3 gene consists of the sequence set forth in SEQ ID NO: 99. In some aspects, a gRNA that can be used to target the NR4A3 gene consists essentially of the sequence set forth in SEQ ID NO: 99.

As described herein, in some aspects, the gene editing methods can further comprise reducing the level of (i) NR4A1 gene and/or NR4A1 protein, (ii) NR4A2 gene and/or NR4A2 protein, or (iii) both (i) and (ii). In some aspects, a gRNA that can be used to target the NR4A1 gene comprises, consists of, or consists essentially of the sequence set forth in SEQ ID NO: 25. In some aspects, a gRNA that can be used to target the NR4A1 gene comprises the sequence set forth in SEQ ID NO: 25. In some aspects, a gRNA that can be used to target the NR4A1 gene consists of the sequence set forth in SEQ ID NO: 25. In some aspects, a gRNA that can be used to target the NR4A1 gene consists essentially of the sequence set forth in SEQ ID NO: 25. In some aspects, a gRNA that can be used to target the NR4A1 gene comprises, consists of, or consists essentially of the sequence set forth in SEQ ID NO: 26. In some aspects, a gRNA that can be used to target the NR4A1 gene comprises the sequence set forth in SEQ ID NO: 26. In some aspects, a gRNA that can be used to target the NR4A1 gene consists of the sequence set forth in SEQ ID NO: 26. In some aspects, a gRNA that can be used to target the NR4A1 gene consists essentially of the sequence set forth in SEQ ID NO: 26. In some aspects, a gRNA that can be used to target the NR4A2 gene comprises, consists of, or consists essentially of the sequence set forth in SEQ ID NO: 27. In some aspects, a gRNA that can be used to target the NR4A2 gene comprises the sequence set forth in SEQ ID NO: 27. In some aspects, a gRNA that can be used to target the NR4A1 gene consists of the sequence set forth in SEQ ID NO: 27. In some aspects, a gRNA that can be used to target the NR4A1 gene consists essentially of the sequence set forth in SEQ ID NO: 27. In some aspects, a gRNA that can be used to target the NR4A2 gene comprises, consists of, or consists essentially of the sequence set forth in SEQ ID NO: 28. In some aspects, a gRNA that can be used to target the NR4A2 gene comprises the sequence set forth in SEQ ID NO: 28. In some aspects, a gRNA that can be used to target the NR4A1 gene consists of the sequence set forth in SEQ ID NO: 28. In some aspects, a gRNA that can be used to target the NR4A1 gene consists essentially of the sequence set forth in SEQ ID NO: 28. In some aspects, a gRNA that can be used to target the NR4A2 gene comprises, consists of, or consists essentially of the sequence set forth in SEQ ID NO: 29. In some aspects, a gRNA that can be used to target the NR4A2 gene comprises the sequence set forth in SEQ ID NO: 29. In some aspects, a gRNA that can be used to target the NR4A1 gene consists of the sequence set forth in SEQ ID NO: 29. In some aspects, a gRNA that can be used to target the NR4A1 gene consists essentially of the sequence set forth in SEQ ID NO: 29.

As used herein, the term “contacting” (for example, contacting a cell, e.g., an immune cell with at least one gRNA and at least one Cas9) is intended to include incubating at least one gRNA and at least one Cas protein, e.g., Cas9, in the cell together in vitro (e.g., adding the gRNA and/or Cas protein, or nucleic acid(s) encoding the gRNA(s) and/or Cas9 protein(s) to cells in culture) or contacting a cell in vivo or ex vivo.

The step of contacting an NR4A3 gene target sequence with at least one gRNA and at least one Cas protein, e.g., Cas9, as disclosed herein (or at least one nucleic acid encoding them) can be conducted in any suitable manner. For example, the cells, e.g., immune cells, can be treated in cell culture conditions. It is understood that the cells contacted with at least one gRNA and at least one Cas protein, e.g., a Cas9 protein, disclosed herein (or at least one nucleic acid encoding them) can also be simultaneously or subsequently contacted with another agent, e.g., a vector comprising at least one nucleic acid sequence encoding a CAR or a TCR. In some aspects, after the cell has been contacted in vitro or ex vivo, the method further comprises introducing the cell into the subject, thereby treating or ameliorating the symptoms of a disease or condition, e.g., cancer.

For ex vivo methods, cells can include autologous cells, i.e., an immune cell or cells taken from a subject who is in need of altering a target polynucleotide sequence (e.g., the NR4A3 gene) in the cell or cells (i.e., the donor and recipient are the same individual). Autologous cells have the advantage of avoiding any immunologically-based rejection of the cells. Alternatively, the cells can be heterologous, e.g., taken from a donor. Typically, when the cells come from a donor, they will be from a donor who is sufficiently immunologically compatible with the recipient, i.e., will not be subject to transplant rejection, to lessen or remove the need for immunosuppression. In some aspects, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994).

In some aspects, the present disclosure provides a method of generating a modified immune cell comprising altering the NR4A3 gene sequence in a cell, e.g., an immune cell (such as a T cell), ex vivo by contacting the NR4A3 gene sequence in the cell with a Cas9 protein (or a nucleic acid encoding such Cas9 protein) and one gRNA which target motifs in the NR4A3 gene (for example motifs wherein the gRNAs direct the Cas9 protein to the target gene and hybridize to the target motifs, wherein the NR4A3 gene is partially or totally cleaved, and wherein the efficiency of cleavage is from about 10% to about 100%. Non-limiting examples of such gRNAs are provided herein (see, e.g., Tables A, C, and D). As described herein, in some aspects, the method of generating a modified immune cell described herein comprises altering the NR4A gene sequence by contacting the cell with a first nucleic acid molecule encoding the Cas9 protein and a second nucleic acid molecule comprising a gRNA that targets one or more members of the NR4A gene family. In some aspects, the first and nucleic acid molecules are contacted with the cell sequentially. In some aspects, the first and nucleic acid molecules are contacted with the cell concurrently. For instance, in some aspects, the cell is contacted with a single polynucleotide comprising the first nucleic acid molecule encoding the Cas9 protein and the second nucleic acid molecule comprising a gRNA.

In some aspects, the cell has been modified (e.g., transfected) with a nucleic acid (e.g., a vector) encoding a CAR or a TCR previously, subsequently, or concurrently to the altering step described above.

In some aspects, the efficiency of cleavage is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

The CRISPR/Cas system of the present disclosure can use gRNA spacer sequences of varying lengths, depending on the Cas used, e.g., a Cas9. Cas9 from different species must be paired with their corresponding gRNAs to form a functional ribonucleoprotein (RNP) complex, in other words, chimeric gRNA frames engineered from different bacterial species can have different length due to differences in spacer sequence and chimeric frame sequence.

In some aspects, the gRNA spacer sequence can be least 18 nucleotides (e.g., 18, 19, 20, 21, or 22 nucleotides) long. For example, the length of S. pyogenes gRNA spacer sequences in gRNAs binding to S. pyogenes Cas9 is 20 nucleotides, while the length of S. aureus gRNA spacer sequences in gRNAs binding to S. aureus Cas9 is 21 nucleotides. In some aspects, the gRNA spacer sequence can comprise 10, 11, 12, 13, 14, 15, 16, 17, 18, 19, 20, 21, 22, 23, 24, 25, 26, 27, 28, 29, 30, 31, 32, 33, 34 or 35 nucleotides. In a specific aspect, the gRNA comprises a spacer sequence consisting of between 18 and 22 consecutive nucleotides (e.g., 20) corresponding to a subsequence of exon 3 of the NR4A3 gene. In some aspects, the gRNA comprises a spacer sequence consisting of between 18 and 22 consecutive nucleotides (e.g., 20) corresponding to a subsequence of exon 4 of the NR4A3 gene. In some aspects, the gRNA comprises a spacer sequence consisting of between 18 and 22 consecutive nucleotides (e.g., 20) corresponding to a subsequence of the exon 3 or exon 4 of the NR4A3 gene.

Although a perfect match between the gRNA spacer sequence and the DNA strand to which it binds on the NR4A3 gene is preferred, a mismatch between a gRNA spacer sequence and a NR4A3 target sequence is also permitted as along as it still results in a reduction of NR4A3 gene levels or a decrease in NR4A3 gene function. A “seed” sequence of between 8-12 consecutive nucleotides on the gRNA perfectly complementary to the target NR4A3 sequence is preferred for proper recognition of the target sequence on the NR4A3 gene. The remainder of the gRNA spacer sequence can comprise one or more mismatches.

In general, gRNA activity is inversely correlated with the number of mismatches. Preferably, the gRNA spacer sequences of the present disclosure comprise less than 7 mismatches. In some aspects, gRNA spacer sequence comprises 7 mismatches, 6 mismatches, 5 mismatches, 4 mismatches, 3 mismatches, more preferably 2 mismatches, or less, and even more preferably no mismatch, with the corresponding NR4A3 gene target sequence. The smaller the number of nucleotides in the gRNA the smaller the number of mismatches tolerated. The binding affinity is thought to depend on the sum of matching gRNA-DNA combinations.

The gRNA spacer sequences of the present disclosure can be selected to minimize off-target effects of the CRISPR/Cas editing system. Accordingly, in some aspects, the gRNA spacer sequence is selected such that it contains at least two mismatches when compared with all other genomic nucleotide sequences in the cell. In some aspects, the gRNA spacer sequence is selected such that it contains at least one mismatch when compared with all other genomic nucleotide sequences in the cell. Those skilled in the art will appreciate that a variety of techniques can be used to select suitable gRNA spacer sequences for minimizing off-target effects (e.g., bioinformatics analyses).

In some aspects, the gRNA spacer sequence comprises, consists, or consists essentially of a spacer sequence of SEQ ID NO: 31-42.

In some aspects, the gRNA spacer sequence comprises, consists, or consists essentially of a spacer sequence comprising at least one, two, three, four or five nucleotide mismatches compared to a DNA sequence of any one of SEQ ID NOS: 31-42.

In some aspects, editing efficacy can be increased by targeting multiple location. Accordingly, in some aspects, the methods disclosed herein comprise using one gRNA targeting a location upstream from exon 1 of the NR4A3 gene. In some aspects, the methods disclosed herein comprise using 2, 3, 4, 5, 6, 7, 8, 9 or 10 gRNAs targeting locations upstream from exon 1 of the NR4A3 gene. Also, in some aspects, the methods disclosed herein comprise using one gRNA targeting a location downstream from exon 4 of the NR4A3 gene. In some aspects, the methods disclosed herein comprise using 2, 3, 4, 5, 6, 7, 8, 9 or 10 gRNAs targeting locations downstream from exon 4 of the NR4A3 gene.

In some aspects, two gRNAs are complementary to and/or hybridize to sequences on the same strand of the NR4A3 gene. In some aspects, two gRNAs are complementary to and/or hybridize to sequences on the opposite strands of the NR4A3 gene. In some aspects, the two gRNAs are not complementary to and/or do not hybridize to sequences on the opposite strands of the NR4A3 gene. In some aspects, two gRNAs are complementary to and/or hybridize to overlapping target motifs of the NR4A3 gene. In some aspect, two gRNAs are complementary to and/or hybridize to offset target motifs of the NR4A3 gene.

In general, the gRNAs of the present disclosure can comprise any variant of its sequence or chemical modifications provided that it allows for the binding of the corresponding Cas protein, e.g., a Cas9 protein, to a target sequence, and subsequent ablation (total or partial) of the NR4A3 gene.

The Cas proteins, e.g., Cas9, used in the methods disclosed herein are endonucleases that cleave nucleic acids and are encoded by the CRISPR loci of numerous bacterial genomes and is involved in the Type II CRISPR system. Cas9 proteins are produced by numerous species of bacteria including Streptococcus pyogenes, Staphylococcus aureus, Streptococcus thermophilus, Neisseria meningitidis, etc. Accordingly, the Cas9 protein useful for the present disclosure can be derived from any suitable bacteria known in the art. Non-limiting examples of such bacteria include Streptococcus pyogenes, Streptococcus mutans, Streptococcus pneumonia, Streptococcus aureus, Streptococcus thermophilus, Campylobacter jejuni, Neisseria meningitidis, Pasteurella multocida, Listeria innocua, and Francisella novicida. The methods disclosed herein can be practiced with any Cas9 known in the art. In some aspects, the Cas9 is a wild type Cas9. In some aspects, the Cas9 is a mutated Cas9 with enhanced enzymatic activity or a fusion protein comprising a Cas9 moiety. In some aspects, the Cas9 nuclease protein is Streptococcus pyogenes Cas9 protein.

Because Cas9 nuclease proteins are normally expressed in bacteria, it can be advantageous to modify their nucleic acid sequences for optimal expression in eukaryotic cells (e.g., mammalian cells) when designing and preparing Cas9 recombinant proteins. Accordingly, in some aspects, the nucleic acid encoding a Cas9 used in the methods disclosed herein has been codon optimized for expression in eukaryotic cells, e.g., for expression in cell of a human subject in need thereof.

In some aspects, a Cas9 protein used in the methods disclosed herein comprises one or more amino acid substitutions or modifications. In some aspects, the one or more amino acid substitutions comprises a conservative amino acid substitution. In some instances, substitutions and/or modifications can prevent or reduce proteolytic degradation and/or extend the half-life of the polypeptide in a cell. In some aspects, the Cas9 protein can comprise a peptide bond replacement (e.g., urea, thiourea, carbamate, sulfonyl urea, etc.). In some aspects, the Cas9 protein can comprise a naturally occurring amino acid. In some aspects, the Cas9 protein can comprise an alternative amino acid (e.g., D-amino acids, beta-amino acids, homocysteine, phosphoserine, etc.). In some aspects, the Cas9 protein can comprise a modification to include a heterologous moiety (e.g., PEGylation, glycosylation, lipidation, acetylation, end-capping, etc.).

Although the methods disclosed herein are generally practiced using Cas9 proteins, it is envisioned that in some aspects, the Cas protein can be a Cas1, Cas2, Cas3, Cas4, Cas5, Cas6, Cas7, or Cas8. In some aspects, the Cas protein is Cas9 protein from any bacterial species or functional portion thereof. In some specific aspects, the Cas9 protein used in the methods disclosed herein is a Streptococcus pyogenes or Staphylococcus aureus Cas9 protein or a functional portion thereof, or a nucleic acid encoding such Cas9 or functional portion thereof. Non-limiting examples of other Cas nucleases that can be used are known in the art and described in, e.g., U.S. Pat. No. 9,970,001 B2; U.S. Pat. No. 10,221,398 B2; and US 2020/0190487 A1, each of which is incorporated herein by reference in its entirety. In some aspects, a Cas nuclease useful for the present disclosure comprises a Type I Cas protein. Non-limiting examples of Type I Cas proteins include Cas3, Cas5, Cas6, Cas7, Cas8a, Cas8b, Cas8c, Cas10d, Cse1, Cse2, Csy1, Csy2, Csy3, and variants thereof. In some aspects, a Cas nuclease useful for the present disclosure comprises a Type II Cas protein. Non-limiting examples of Type II Cas proteins include Cas9, Csn2, Cas4, and variants thereof. In some aspects, a Cas nuclease useful for the present disclosure comprises a Type III Cas protein. Non-limiting examples include Cas10, Csm2, Cmr5, Csx10, Csx11, and variants thereof. In some aspects, a Cas nuclease useful for the present disclosure comprises a Type IV Cas protein. Non-limiting example of such a Cas protein includes Csfl. In some aspects, a Cas nuclease useful for the present disclosure comprises a Type V Cas protein. Non-limiting examples include, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, and variants thereof. In some aspects, a Cas nuclease useful for the present disclosure comprises a Type VI Cas protein. Non-limiting examples of Type VI Cas proteins include Cas13, Cas13a (C2c2), Cas13b, Cas13c, Cas13d, and variants thereof.

In some cases, a Cas protein useful for the present disclosure comprises orthologues or homologues of the above mentioned Cas proteins. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins can but need not be structurally related, or are only partially structurally related. An “orthologue” of a protein as used herein is a protein of a different species which performs the same or a similar function as the protein it is an orthologue of. Orthologous proteins can but need not be structurally related, or are only partially structurally related.

As used herein, “functional portion” refers to a portion of a peptide, e.g., Cas9, which retains its ability to complex with at least one gRNA and cleave a target sequence, resulting in reduced expression of the NR4A3 gene and/or NR4A3 protein. In some aspects, the functional portion comprises a combination of operably linked Cas9 protein functional domains selected from the group consisting of a DNA binding domain, at least one RNA binding domain, a helicase domain, and an endonuclease domain. In some aspects, the functional domains form a non-covalent complex. In some aspects, the functional domains form a fusion complex (e.g., a fusion protein). In some aspects, the functional domains are chemically linked (e.g., through one or more spacers or linkers). In some aspects, the functional domains are conjugated.

It should be appreciated that the present disclosure contemplates various ways of contacting the NR4A3 gene with at least one gRNA and at least one Cas protein, e.g., Cas9. In some aspects, exogenous Cas protein, e.g., Cas9, can be introduced into the cell in polypeptide form. In some aspects, a Cas protein, e.g., Cas9, can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In some aspects, Cas protein, e.g., Cas9, can be conjugated to or fused to a charged protein, e.g., a protein that carries a positive, negative or overall neutral electric charge. Such linkage can be covalent. In some aspects, the Cas protein, e.g., Cas9, can be fused to a superpositively charged peptide to significantly increase the ability of the Cas protein, e.g., Cas9, to penetrate a cell. See Cronican et al. ACS Chem. Biol. 5(8):747-52 (2010). In some aspects, the Cas protein, e.g., Cas9, can be fused to a protein transduction domain (PTD) to facilitate its entry into a cell. Exemplary PTDs include, but are not limited to, Tat, oligoarginine, and penetratin. Thus, in some specific aspects, the methods disclosed herein can be practiced using a Cas protein, e.g., a Cas9 protein, comprising a Cas protein fused to a cell-penetrating peptide, a Cas protein fused to a PTD, a Cas protein fused to a tat domain, a Cas protein fused to an oligoarginine domain, a Cas protein fused to a penetratin domain, or a combination thereof.

In some aspects, the Cas protein, e.g., Cas9, can be introduced into a cell, e.g., an immune cell disclosed herein, e.g., an immune cell expressing a CAR or TCR, containing the target polynucleotide sequence, i.e., the NR4A3 gene, in the form of a nucleic acid encoding the Cas protein, e.g., Cas9. The process of introducing the nucleic acids into cells can be achieved by any suitable technique. Suitable techniques include calcium phosphate or lipid-mediated transfection, electroporation, and transduction or infection using a viral vector. In some aspects, the nucleic acid comprises DNA. In some aspects, the nucleic acid comprises a modified DNA, as described herein. In some aspects, the nucleic acid comprises mRNA. In some aspects, the nucleic acid comprises a modified mRNA, as described herein (e.g., a synthetic, modified mRNA).

The gRNA sequences and/or nucleic sequences encoding Cas9 used in the methods disclosed herein can be chemically modified to enhance, for example, their stability (e.g., to increase their plasma half-life after administration to a subject in need thereof). Possible chemical modifications to the gRNAs disclosed herein and/or nucleic sequences encoding, e.g., Cas9, are discussed in detail below in this specification.

In some aspects, the entire gRNA is chemically modified. In some aspects, only the gRNA spacer is chemically modified. In some aspects, the gRNA spacer and gRNA frame sequence are chemically modified. Non-limiting examples of specific chemical modifications are disclosed in detail below.

Accordingly, in some aspects of the methods disclosed herein, the Cas protein (e.g., Cas9) and one or more gRNAs are provided to a target cell through expression from one or more delivery vectors coding therefor. In some aspects, the above-mentioned vector or vectors for introducing the gRNA or gRNAs and Cas9 in a target cell are viral vectors. In some aspects, the above-mentioned vector or vectors for introducing the gRNA or gRNAs and Cas9 in a target cell are non-viral vectors. In some aspects, the viral vector is an adeno-associated vector (AAV), a lentiviral vector (LV), a retroviral vector, an adenovirus vector, a herpes virus vector, or a combination thereof. The AAV vector or vectors can be based on one or more of several capsid types, including AAV1, AAV2, AAV5, AAV6, AAV8, and AAV9. In some aspects, the AAV vector is AAVDJ-8, AAV2DJ9, or a combination thereof.

In addition to the method disclosed above, the present disclosure further provides compositions to practice the disclosed methods. Accordingly, the present disclosure provides a nucleic acid encoding at least one the above-mentioned gRNAs.

Also provided is a composition and/or at least one Cas9. In some aspects, the nucleic acid encoding Cas9 encodes (i) a Cas9 from S. aureus, (ii) a Cas9 from S. pyogenes, (iii) a mutant Cas9 derived from Cas9 from S. aureus or from Cas9 from S. pyogenes wherein the mutant protein retains Cas9 activity, (iv) a fusion protein comprising a Cas9 moiety, or (v) a combination thereof.

In some aspects, one or more gene editing tools (e.g., those disclosed herein) can be used to modify the cells of the present disclosure.

### V.A.2. TALEN

In some aspects, a gene editing tool that can be used to edit (e.g., reduce or inhibit) the expression of NR4A3 gene and/or NR4A3 protein is a nuclease agent, such as a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al., (2010) PNAS 10.1073/pnas.1013133107; Scholze & Boch (2010) Virulence 1:428-432; Christian et al., Genetics (2010) 186:757-761; Li et al., (2010) Nuc. Acids Res. (2010) doi:10.1093/nar/gkg704; and Miller et al., (2011) Nature Biotechnology 29:143-148; all of which are herein incorporated by reference in their entirety.

Non-limiting examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference).

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

In some aspects, each monomer of the TALEN comprises 12-25 TAL repeats, wherein each TAL repeat binds a 1 bp subsite. In some aspects, the nuclease agent is a chimeric protein comprising a TAL repeat-based DNA binding domain operably linked to an independent nuclease. In some aspects, the independent nuclease is a FokI endonuclease. In some aspects, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In some aspects, the nuclease agent comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

### V.A.3. Zinc Finger Nuclease (ZFN)

In some aspects, a gene editing tool useful for the present disclosure includes a nuclease agent, such as a zinc-finger nuclease (ZFN) system. Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence (e.g., NR4A3), directs the activity of the enzymatic domain to the vicinity of the sequence and, hence, induces modification of the endogenous target gene in the vicinity of the target sequence. A zinc finger domain can be engineered to bind to virtually any desired sequence. As disclosed herein, in some aspects, the zinc finger domain binds a DNA sequence that encodes the NR4A3 protein. Accordingly, after identifying a target genetic locus containing a target DNA sequence at which cleavage or recombination is desired (e.g., a target locus in a target gene referenced in Table 1), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some aspects, a zinc finger binding domain comprises one or more zinc fingers. Miller et al., (1985) EMBO J. 4:1609-1614; Rhodes (1993) Scientific American February:56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However, binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al., (1997) Nucleic Acids Res. 25:3379-3388. Additional enzymes which cleave DNA are known (e.g., 51 Nuclease; mung bean nuclease; pancreatic DNaseI; micrococcal nuclease; yeast HO endonuclease; see also Linn et al., (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al., (1992) Proc. Natl. Acad. Sci. USA 89:4275-4279; Li et al., (1993) Proc. Natl. Acad. Sci. USA 90:2764-2768; Kim et al., (1994a) Proc. Natl. Acad. Sci. USA 91:883-887; Kim et al., (1994b) J. Biol. Chem. 269: 31,978-31,982. Thus, in some aspects, fusion proteins comprise the enzymatic domain from at least one Type IIS restriction enzyme and one or more zinc finger binding domains.

An exemplary Type IIS restriction enzyme, whose cleavage domain is separable from the binding domain, is FokI. This particular enzyme is active as a dimer. Bitinaite et al., (1998) Proc. Natl. Acad. Sci. USA 95: 10,570-10,575. Thus, for targeted double-stranded DNA cleavage using zinc finger-FokI fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

### V.A.3. Meganucleases

In some aspects, a gene editing tool that be used to regulate NR4A3 expression in a cell includes a nuclease agent such as a meganuclease system. Meganucleases have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see, for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) JMol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346; each of which is herein incorporated by reference in its entirety.

Any meganuclease can be used herein, including, but not limited to, I-SceI, I-SceII, I-SceTII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In some aspects, the meganuclease recognizes double-stranded DNA sequences of 12 to 40 base pairs. In some aspects, the meganuclease recognizes one perfectly matched target sequence in the genome. In some aspects, the meganuclease is a homing nuclease. In some aspects, the homing nuclease is a “LAGLIDADG” family of homing nuclease. In some aspects, the “LAGLIDADG” family of homing nuclease is selected from I-SceI, I-CreI, I-Dmol, or combinations thereof.

### V.A.4. Restriction Endonucleases

In some aspects, a gene editing tool useful for the present disclosure includes a nuclease agent such as a restriction endonuclease, which includes Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type IIs enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

As described herein, in some aspects, a gene editing tool (e.g., CRISPR, TALEN, meganuclease, restriction endonuclease, RNAi, antisense oligonucleotides) can be introduced into the cell by any means known in the art. In some aspects, the polypeptide encoding the particular gene editing tool can be directly introduced into the cell. Alternatively, a polynucleotide encoding the gene editing tool can be introduced into the cell. In some aspects, when a polynucleotide encoding the gene editing tool is introduced into the cell, the gene editing tool can be transiently, conditionally or constitutively expressed within the cell. Thus, the polynucleotide encoding the gene editing tool can be contained in an expression cassette and be operably linked to a conditional promoter, an inducible promoter, a constitutive promoter, or a tissue-specific promoter. Alternatively, the gene editing tool is introduced into the cell as an mRNA encoding or comprising the gene editing tool.

Active variants and fragments of nuclease agents (i.e., an engineered nuclease agent) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus in some aspects, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example the polynucleotide encoding the nuclease agent can be modified to substitute codons having a higher frequency of usage in a given cell of interest.

### V.A.5. Interference RNA (RNAi)

In some aspects, a gene editing tool that can be used to reduce the expression of NR4A3 in a cell includes an RNA interference molecule (“RNAi”). As used herein, RNAi are RNA polynucleotide that mediates the decreased the expression of an endogenous target gene product by degradation of a target mRNA through endogenous gene silencing pathways (e.g., Dicer and RNA-induced silencing complex (RISC)). Non-limiting examples of RNAi agents include micro RNAs (also referred to herein as “miRNAs”), short hair-pin RNAs (shRNAs), small interfering RNAs (siRNAs), RNA aptamers, or combinations thereof.

In some aspects, the gene editing tools useful for the present disclosure comprises one or more miRNAs. “miRNAs” refer to naturally occurring, small non-coding RNA molecules of about 21-25 nucleotides in length. In some aspects, the miRNAs useful for the present disclosure are at least partially complementary to a NR4A3 mRNA molecule. miRNAs can downregulate (e.g., decrease) expression of an endogenous target gene product (i.e., NR4A3 protein) through translational repression, cleavage of the mRNA, and/or deadenylation.

In some aspects, a gene editing tool that can be used with the present disclosure comprises one or more shRNAs. “shRNAs” (or “short hairpin RNA” molecules) refer to an RNA sequence comprising a double-stranded region and a loop region at one end forming a hairpin loop, which can be used to reduce and/or silence a gene expression. The double-stranded region is typically about 19 nucleotides to about 29 nucleotides in length on each side of the stem, and the loop region is typically about three to about ten nucleotides in length (and 3′- or 5′-terminal single-stranded overhanging nucleotides are optional). shRNAs can be cloned into plasmids or in non-replicating recombinant viral vectors to be introduced intracellularly and result in the integration of the shRNA-encoding sequence into the genome. As such, an shRNA can provide stable and consistent repression of endogenous target gene (i.e., NR4A3) translation and expression.

In some aspects, a gene editing tool disclosed herein comprises one or more siRNAs. “siRNAs” refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA (i.e., NR4A3 mRNA), resulting in specific gene silencing. In some aspects, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequence (i.e., NR4A3 mRNA). siRNAs and shRNAs are further described in Fire et al., Nature 391:19, 1998 and U.S. Pat. Nos. 7,732,417; 8,202,846; and 8,383,599; each of which is herein incorporated by reference in its entirety.

### V.A.6. Antisense Oligonucleotides (ASO)

In some aspects, a gene editing tool that can be used to reduce the expression of NR4A3 gene and/or NR4A3 protein in a cell includes antisense oligonucleotides. As used herein, “antisense oligonucleotide” or “ASO” refer to an oligonucleotide capable of modulating expression of a target gene (i.e., NR4A3) by hybridizing to a target nucleic acid, in particular to a contiguous sequence on a target nucleic acid. Antisense oligonucleotides are not essentially double stranded and are therefore not siRNAs or shRNAs.

In some aspects, ASOs useful for the present disclosure are single stranded. It is understood that single stranded oligonucleotides of the present disclosure can form hairpins or intermolecular duplex structures (duplex between two molecules of the same oligonucleotide), as long as the degree of intra or inter self-complementarity is less than approximately 50% across of the full length of the oligonucleotide. In some aspects, ASOs useful for the present disclosure can comprise one or more modified nucleosides or nucleotides, such as 2′ sugar modified nucleosides. Additional modifications that can be made to an ASO (e.g., such as those that can be used to inhibit or reduce NR4A3 gene expression) are provided in, e.g., US Publ. No. 2019/0275148 A1.

In some aspects, ASOs can reduce the expression of NR4A3 protein via nuclease mediated degradation of the NR4A3 transcript (e.g., mRNA), where the ASOs are capable of recruiting a nuclease, e.g., RNase H, such as RNaseHl. RNase H is a ubiquitous enzyme that hydrolyzes the RNA strand of an RNA/DNA duplex. Accordingly, in some aspects, once bound to the target sequence (e.g., NR4A3 mRNA), ASOs can induce the degradation of the NR4A3 mRNA and thereby, reduce the expression of NR4A3 protein.

As disclosed herein, the above examples of gene editing tools are not intended to be limiting and any gene editing tool available in the art can be used to reduce or inhibit the expression of NR4A3 gene and/or NR4A3 protein. Additionally, as described herein, where the level of (i) NR4A1 gene and/or NR4A1 protein, (ii) NR4A2 gene and/or NR4A2 protein, or (iii) both (i) and (ii) are also reduced, in some aspects, any of the above-described gene editing tools that specifically target the NR4A1 and/or NR4A2 gene can also be used.

### IV.A.7. Repressors

In some aspects, a gene editing tool that can be used with the present disclosure (e.g., to reduce the expression of NR4A1, NR4A2, and/or NR4A3 gene and/or protein) comprises a repressor. As used herein, the term “repressor” refers to any agent that is capable of binding to the following NR4A response elements without activating transcription: (i) NGFI-B response element (NBRE), (ii) Nur-response element (NurRE), or (iii) both (i) and (ii). Accordingly, by binding to NBRE and/or NurRE, the repressors described herein are capable of repressing (or reducing or inhibiting) the level of one or more NR4A family members in a cell (e.g., immune cell expressing a CAR or TCR). In some aspects, the binding of the repressor to NBRE and/or NurRE reduces the level of a NR4A1 gene and/or NR4A1 protein in a cell when the cell is contacted with the repressor. In some aspects, the binding of the repressor to NBRE and/or NurRE reduces the level of a NR4A2 gene and/or NR4A2 protein in a cell when the cell is contacted with the repressor. In some aspects, the binding of the repressor to NBRE and/or NurRE reduces the level of a NR4A3 gene and/or NR4A3 protein in a cell when the cell is contacted with the repressor. In some aspects, the binding of the repressor to NBRE and/or NurRE reduces the level of both (i) a NR4A1 gene and/or NR4A1 protein and (ii) a NR4A2 gene and/or NR4A2 protein. In some aspects, the binding of the repressor to NBRE and/or NurRE reduces the level of both (i) a NR4A1 gene and/or NR4A1 protein and (ii) a NR4A3 gene and/or NR4A3 protein. In some aspects, the binding of the repressor to NBRE and/or NurRE reduces the level of both (i) a NR4A2 gene and/or NR4A2 protein and (ii) a NR4A3 gene and/or NR4A3 protein. In some aspects, the binding of the repressor to NBRE and/or NurRE reduces the level of each of the following: (i) a NR4A1 gene and/or NR4A1 protein, (ii) a NR4A2 gene and/or NR4A2 protein, and (iii) a NR4A3 gene and/or NR4A3 protein. Repressors that are capable of reducing the level of all members of the NR4A family (i.e., NR4A1, NR4A2, and NR4A3) are also known as “NR4A super-repressors.” See, e.g., WO2020237040A1, which is incorporated herein by reference in its entirety.

As is apparent from at least the above disclosure, repressors that are useful for the present disclosure comprises a DNA-binding domain that is capable of binding to the NBRE and/or NurRE response elements. In some aspects, such repressors comprise additional domains. Non-limiting examples of such additional domains include: NR4A ligand-binding domain, FLAG domain, Kruppel-associated box (KRAB) domain, NCOR domain, T2A domain, self-cleavage domain, nuclear localization signal, dimerization domain (e.g., diZIP dimerization domain), transcriptional repressor domain, chromatin compaction domain, an epitope tag, or any combination thereof. Additional disclosure relating to such additional domains can be found, e.g., in WO2020237040A1, which is incorporated herein by reference in its entirety. In some aspects, the additional domains do not comprise a transcriptional activation domain.

As described herein, in some aspects, in reducing the level of one or more members of the NR4A family, a cell can be contacted with a NR4A repressor protein described herein. In some aspects, a cell is contacted with a nucleic acid sequence encoding a NR4A repressor.

### V.B. Methods of Reducing Exhaustion/Dysfunction

The present disclosure provides methods of reducing, ameliorating, or inhibiting exhaustion or dysfunction of a cell comprising modifying the cell to express reduced levels of NR4A3 gene and/or NR4A3 protein, while having endogenous expression of NR4A1 and NR4A2 genes and NR4A1 and NR4A2 proteins. As described herein, in some aspects, such cells (i.e., modified to have reduced level of NR4A3 gene and/or NR4A3 protein) can be further modified to have reduced level (i) NR4A1 gene and/or NR4A1 protein, (ii) NR4A2 gene and/or NR4A2 protein, or (iii) both (i) and (ii).

One of the various ways that tumor cells can evade a host immune response is by causing tumor-specific immune cells, e.g., T cells, to become exhausted. As used herein, the term “exhaustion,” or more specifically, “T cell exhaustion,” refers to the loss of T cell function, which can occur as a result of an infection or a disease (e.g., cancer). T cell exhaustion can be used interchangeably with “T cell dysfunction” or “T cell anergy” in the present disclosure. In some aspects, T cell exhaustion is associated with increased expression of various immune checkpoint inhibitory molecules (e.g., PD-1, TIM-3, and LAG-3), apoptosis, and reduced effector function (e.g., cytokine production and expression of cytotoxic molecules, such as perforin and granzymes). Accordingly, the terms “reduce T cell exhaustion,” “ameliorate T cell exhaustion,” “inhibit T cell exhaustion,” and the like, refers to a condition of restored functionality of T cells characterized by one or more of the following: (i) decreased expression of one or more immune checkpoint inhibitory molecules (e.g., PD-1, TIM-3, and LAG-3), (ii) increased memory formation and/or maintenance of memory markers (e.g., CD45RO, CD62L, and/or CCR7), (iii) prevention of apoptosis, (iv) increased cytokine production (e.g., IL-2, IFN-γ, and/or TNF-α), (v) enhanced killing capacity, (vi) increased recognition of tumor targets with low surface antigen, (vii) enhanced proliferation in response to antigen, and (viii) any combination thereof.

In some aspects, modifying a cell, e.g., in a method disclosed herein, comprises modifying an immune cell, e.g., T cell, to have resistance or tolerance to exhaustion. Accordingly, in some aspects, the present disclosure relates to methods of reducing exhaustion in an immune cell, e.g., T cell (e.g., tumor-specific T cell) by reducing the expression levels of NR4A3 gene and/or NR4A3 protein (alone or in combination with reduced level of the NR4A1 gene and/or NR4A1 protein and/or the NR4A2 gene and/or NR4A2 protein) in the cell. In some aspects, reducing immune cell, e.g., T cell, exhaustion comprises reversing the dysfunction that has already occurred in the immune cell, e.g., T cell, (i.e., making exhausted T cells become less exhausted). In some aspects, reducing immune cell, e.g., T cell, exhaustion comprises preventing a newly activated immune cell, e.g., T cell, from becoming exhausted. As is apparent from the present disclosure, in some aspects, the immune cell is previously, concurrently, or subsequently modified, e.g., to express a ligand binding protein (e.g., CAR or TCR).

In some aspects, reducing immune cell, e.g., T cell, exhaustion comprises both reversing and preventing exhaustion in an immune cell, e.g., a T cell.

The exhaustion state of an immune cell, e.g., a T cell, can be determined by various methods known in the art. In some aspects, the exhaustion state of an immune cell, e.g., a T cell, can be measured by evaluating the resistance of the immune cell, e.g., a T cell, to apoptosis. Accordingly, in some aspects, the cell composition of the disclosure (i.e., expressing reduced levels of NR4A3 gene and/or NR4A3 protein and (i) endogenous levels of NR4A1 and NR4A2 genes and NR4A1 and NR4A2 proteins, (ii) reduced level of NR4A1 gene and/or NR4A1 protein, (iii) reduced level of NR4A2 gene and/or NR4A2 protein, or (iv) reduced level of both the NR4A1 gene and/or NR4A1 protein and the NR4A2 gene and/or NR4A2 protein) exhibits increased resistance to apoptosis. In some aspects, the resistance to apoptosis in the cell composition of the disclosure is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., apoptosis of a corresponding cell, e.g., an immune cell such as a T cell, that has not been modified to express lower levels of NR4A3 gene and/or NR4A3 protein). In some aspects, increased resistance to apoptosis can promote the long-term persistence or survival of the T cell. Therefore, in some aspects, the cell composition of the disclosure (i.e., expressing reduced levels of NR4A3 gene and/or NR4A3 protein, alone or in combination with reduced level of the NR4A1 gene and/or NR4A1 protein and/or the NR4A2 gene and/or NR4A2 protein) exhibits enhanced persistence or survival compared to a reference cell (e.g., corresponding cell, e.g., an immune cell such as a T cell, that has not been modified to express reduced levels of NR4A3 gene and/or NR4A3 protein).

In some aspects, the persistence or survival in the cell composition of the disclosure is increased by at least about 2 fold to about 100 fold. In certain aspects, the persistence or survival in the cell composition of the disclosure is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to the reference cell (e.g., corresponding cell, e.g., an immune cell such as a T cell, that has not been modified to express reduced levels of NR4A3 gene and/or NR4A3 protein).

In some aspects, the exhaustion state of an immune cell, e.g., a T cell, can be measured by evaluating the resistance of the immune cell, e.g., a T cell, to immune checkpoint molecules. In some aspects, the resistance to immune checkpoint molecules is increased in the cell composition of the disclosure by at least 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., resistance in a corresponding cell, e.g., an immune cell such as a T cell, that has not been modified to express reduced levels of NR4A3 gene and/or NR4A3 protein).

Not to be bound by any one theory, in some aspects, the increased resistance to immune checkpoint molecules is due to decreased expression of one or more immune checkpoint molecules on the immune cell, e.g., a T cell. Accordingly, in some aspects, the cell composition of the disclosure expresses reduced levels of one or more immune checkpoint molecules compared to a reference cell (e.g., corresponding cell, e.g., an immune cell such as a T cell, that has not been modified to express reduced levels of NR4A3 gene and/or NR4A3 protein). In some aspects, the expression level of an immune checkpoint molecule is reduced in the cell composition of the disclosure by at least about 5%, at least about 10%, at least about 15%, at least about 30%, at least about 40%, at least about 50%, at least about 60%, at least about 70%, at least about 80%, at least about 90%, or at least about 100% compared to the reference cell. Examples of immune checkpoint molecules are known in the art and include, but are not limited to, PD-1, TIM-3, LAG-3, BTLA, SIGLEC7, CD200R, TIGIT, VISTA, and any combination thereof.

In some aspects, the exhaustion state of an immune cell, e.g., a T cell, can be measured by evaluating the ability of the immune cell, e.g., a T cell, to produce cytokines upon stimulation, e.g., T-cell receptor (TCR) stimulation. Accordingly, in some aspects, the cell composition of the disclosure (i.e., expressing reduced levels of NR4A3 gene and/or NR4A3 protein alone or in combination with reduced level of the NR4A1 gene and/or NR4A1 protein and/or the NR4A2 gene and/or NR4A2 protein) exhibits increased cytokine production compared to a reference (e.g., cytokine production in a corresponding cell, e.g., an immune cell such as a T cell, that has not been modified to express reduced levels of NR4A3 gene and/or NR4A3 protein).

In some aspects, cytokine production in the cell composition of the disclosure is increased by at least about 2 fold, 8 fold, 10 fold, 15 fold, 20 fold, 25 fold, 30 fold, 35 fold, 40 fold, 45 fold, 50 fold, 100 fold or more. In some aspects, cytokine production in the cell composition of the disclosure is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference (e.g., cytokine production in a corresponding cell, e.g., an immune cell such as a T cell, that has not been modified to express reduced levels of NR4A3 gene and/or NR4A3 protein). Non-limiting examples of cytokines include IFN-γ, IL-2, TNF-α, GM-CSF, IL-6, IL-10, IL-4, IL-5, IL-8, IL-9, IL-13, IL-17, IL-22, CCL2, CCL3, and any combination thereof.

In some aspects, the exhaustion state of an immune cell, e.g., a T cell, can be measured by evaluating the ability of the immune cell, e.g., a T cell, to kill tumor cells after repeated tumor challenge. In some aspects, the cell composition of the disclosure exhibits increased tumor cell killing compared to a reference cell (e.g., corresponding cell, e.g., an immune cell such as a T cell, that has not been modified to express reduced levels of NR4A3 gene and/or NR4A3 protein) after two tumor challenges. In some aspects, the cell composition of the disclosure exhibits increased tumor cell killing compared to a reference cell (e.g., corresponding cell, e.g., an immune cell such as a T cell, that has not been modified to express reduced levels of NR4A3 gene and/or NR4A3 protein) after three tumor challenges. In some aspects, the cell composition of the disclosure exhibits increased tumor cell killing compared to a reference cell (e.g., corresponding cell, e.g., an immune cell such as a T cell, that has not been modified to express reduced levels of NR4A3 gene and/or NR4A3 protein) after four tumor challenges. In some aspects, the cell composition of the disclosure exhibits increased tumor cell killing compared to a reference cell (e.g., corresponding cell, e.g., an immune cell such as a T cell, that has not been modified to express reduced levels of NR4A3 gene and/or NR4A3 protein) after five tumor challenges. In some aspects, killing tumor cells comprises preventing the outgrowth of tumor cells.

In some aspects, after each of the tumor challenges, the ability to kill tumor cells of the cell composition of the disclosure is increased by about 2 fold, 8 fold, 10 fold, 15 fold, 20 fold, 25 fold, 30 fold, 35 fold, 40 fold, 45 fold, 50 fold, 100 fold or more. In some aspects, the ability to kill tumor cells is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference (e.g., tumor cell killing in a corresponding cell, e.g., an immune cell such as a T cell, that has not been modified to express reduced levels of NR4A3 gene and/or NR4A3 protein).

### V.C. Method of Maintaining Anti-Tumor Function in Tumor Microenvironment

Tumorigenesis (i.e., the production or formation of a tumor) is a complex and dynamic process consisting of three stages: initiation, progression, and metastasis. Each of these stages is tightly regulated by tumor microenvironment (TME). See Wang, M., et al., J Cancer 8(5):761-773 (2017). As used herein, the term “tumor microenvironment” or “TME” refers to the environment around a tumor, including the surrounding blood vessels, immune cells, fibroblasts, signaling molecules, and the extracellular matrix. As described further below, tumors can influence the microenvironment by releasing extracellular signals, promoting tumor angiogenesis, and inducing peripheral immune tolerance, while the immune cells in the microenvironment can affect the growth and evolution of the tumor cells.

Tumor cells generally grow at very high speeds leading to insufficient blood supply to the tumor microenvironment. See Gouirand, V., et al., Front Oncol 8:117 (2018). This results in the tumor microenvironment to become hypoxic and increased generation of reactive oxygen species (ROS). Hypoxia and ROS can negatively affect immune cell function, and thereby, inhibit the anti-tumor immune response in a subject.

In some aspects, modifying a cell, e.g., in a method disclosed herein, results in enhancing the anti-tumor function of an immune cell (e.g., tumor-specific T cell) in a low oxygen environment, such as that found in tumor microenvironment. As used herein, the term “anti-tumor function” refers to the ability of an immune cell (e.g., tumor-specific T cell) to mount an immune response that results in the eradication and/or control of a tumor cell. Non-limiting examples of anti-tumor function comprises cytokine production, proliferation, reduced exhaustion, long-term survival, cytotoxicity (e.g., ability to kill tumor cells), or combinations thereof.

In some aspects, the anti-tumor function of the cell composition of the disclosure (i.e., comprising modified cells with reduced level of NR4A3 gene and/or NR4A3 protein alone or in combination with reduced level of the NR4A1 gene and/or NR4A1 protein and/or the NR4A2 gene and/or NR4A2 protein) is enhanced (i.e., increased) in a low oxygen environment compared to a reference cell (e.g., corresponding cell, e.g., an immune cell such as a T cell, that has not been modified to express reduced levels of NR4A3 gene and/or NR4A3 protein).

The rapid proliferation of tumor cells can also result in the depletion of various nutrients (e.g., glucose) within a tumor microenvironment. See Gouirand, supra. As discussed herein, nutrients, such as glucose, are essential for normal immune cell function and development. In some aspects, the anti-tumor function of the cell composition of the disclosure is enhanced (i.e., increased) in a low nutrient environment compared to a reference cell (e.g., corresponding cell, e.g., an immune cell such as a T cell, that has not been modified to express reduced levels of NR4A3 gene and/or NR4A3 protein).

As described herein, one of the mechanisms by which tumor cells suppress host immune response is by releasing various immunosuppressive metabolites and/or cytokines into tumor microenvironment. The accumulation of such metabolites and/or cytokines within the tumor microenvironment can inhibit the normal function of immune cells (e.g., tumor-infiltrating lymphocytes). Non-limiting examples of such immunosuppressive metabolites and/or cytokines include indolamine-2-3-dioxygenase (IDO), arginase, inducible nitric oxide synthetase (iNOS), lactate dehydrogenase (LDH)-A, TGF-β, IL-10, VEGF, reactive oxygen species (ROS), adenosine, arginase, prostaglandin E2, and combinations thereof.

In some aspects, the anti-tumor function of the cell composition of the disclosure (i.e., comprising modified cells with reduced level of NR4A3 gene and/or NR4A3 protein alone or in combination with reduced level of the NR4A1 gene and/or NR4A1 protein and/or the NR4A2 gene and/or NR4A2 protein) is enhanced (i.e., increased) in the presence of immunosuppressive metabolites and/or cytokines by at least about 2 fold, 8 fold, 10 fold, 15 fold, 20 fold, 25 fold, 30 fold, 35 fold, 40 fold, 45 fold, 50 fold, 100 fold or more. In some aspects, anti-tumor function is increased by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold or more compared to a reference cell (e.g., corresponding cell, e.g., an immune cell such as a T cell, that has not been modified to express reduced levels of NR4A3 gene and/or NR4A3 protein).

Another mechanism by which the tumor microenvironment can inhibit an anti-tumor immune response is through immune suppressive cells, such as myeloid-derived suppressor cells (MDSCs) and regulatory T (T_{reg}) cells.

As used herein, the term “myeloid-derived suppressor cells” or “MDSCs” refer to a heterogeneous population of immune cells that are defined by their myeloid origin, immature state, and ability to potently suppress T cell responses. MDSCs are generated in the bone marrow, and in tumor-bearing hosts, migrate to peripheral lymphoid organs and the tumor to contribute to the formation of the tumor microenvironment. In some aspects, the MDSCs are monocytic MDSCs (M-MDSCs), which are morphologically and phenotypically similar to monocytes. In some aspects, the MDSCs are polymorphonuclear MDSCs (PMN-MDSCs), which are morphologically and phenotypically similar to neutrophils. In some aspects, MDSCs comprise both M-MDSCs and PMN-MDSCs. MDSCs present within the tumor microenvironment generally exhibit poor phagocytic activity, continuous production of reactive oxygen species (ROS), nitric oxide (NO), and mostly anti-inflammatory cytokines (e.g., IL-10 and TGF-β).

As used herein, the term “regulatory T cells” or “T_{reg} cells” refer to a specific population of T cells that have the ability to suppress the proliferation and/or function of other T cells (e.g., tumor-infiltrating lymphocytes). In some aspects, the regulatory T cells are CD4⁺ regulatory T cells. In some aspects, the regulatory T cells are Foxp3⁺. Regulatory T cells can exert their immunosuppressive activity through different contact-dependent and independent mechanisms. Non-limiting examples of such mechanisms include: (i) production of suppressive cytokines (e.g., TGF-β, IL-10, and IL-35); (ii) expression of immune checkpoint and inhibitory receptors (e.g., CTLA-4, PD-L1, Arginase, LAG-3, TIM-3, ICOS, TIGIT, IDO); (iii) direct cytotoxicity (perforin/granzyme-mediated or FasL-mediated); (iv) metabolic disruption of effector T cell activity (e.g., IL-2 consumption); (v) induction of tolerogenic dendritic cells, which can promote T cell exhaustion; (vi) adenosine production, and (vii) any combination thereof.

In some aspects, the anti-tumor function of the cell composition of the disclosure (i.e., comprising modified cells with reduced level of NR4A3 gene and/or NR4A3 protein alone or in combination with reduced level of the NR4A1 gene and/or NR4A1 protein and/or the NR4A2 gene and/or NR4A2 protein) is enhanced (i.e., increased) in the presence of suppressive cells compared to a reference cell (e.g., corresponding cell, e.g., an immune cell such as a T cell, that has not been modified to express reduced levels of NR4A3 gene and/or NR4A3 protein). In some aspects, the suppressive cells are MDSCs. In some aspects, the suppressive cells are T_{reg} cells. In some aspects, the suppressive cells comprise both MDSCs and T_{reg} cells.

While the above disclosure is provided largely in the context of T cells (e.g., tumor-infiltrating lymphocytes), those skilled in the art will recognize that the above disclosure can also apply to other types of immune cells. Accordingly, in some aspects, the methods disclosed herein can be used to enhance the activation, reduce the exhaustion/dysfunction, or maintain the anti-tumor function of any immune cells useful for the treatment of a tumor. Non-limiting examples of such immune cells include a lymphocyte, neutrophil, monocyte, macrophage, dendritic cell, or combinations thereof. In some aspects, a lymphocyte comprises a T cell, tumor-infiltrating lymphocyte (TIL), lymphokine-activated killer cell, natural (NK) cell, or combinations thereof. In some aspects, a lymphocyte is a T cell, e.g., CD4⁺ T cell or a CD8⁺ T cell. In some aspects, a lymphocyte is a tumor infiltrating lymphocyte (TIL). In some aspects, a TIL is a CD8⁺ TIL. In some aspects, a TIL is a CD4⁺ TIL. As described herein, in some aspects, an immune cell of the present disclosure comprises a chimeric antigen receptor (CAR), such as a CAR T cell or a CAR NK cell.

### VI. Nucleic Acids and Vectors

The present disclosure also provides one or more nucleic acid molecules (e.g., polynucleotides) that comprise a gene editing tool for reducing the expression of NR4A3 gene and/or NR4A3 protein (alone or in combination with reduced level of the NR4A1 gene and/or NR4A1 protein and/or the NR4A2 gene and/or NR4A2 protein) in a cell and/or that encode a chimeric antigen receptor or a T cell receptor (e.g., anti-ROR1 CAR or anti-ROR1 TCR) that can be expressed in the modified cell of the present disclosure.

As described herein, some aspects of the present disclosure are directed to polynucleotides (e.g., isolated polynucleotides) comprising a nucleotide sequence that is capable of specifically binding to a target sequence within the NR4A3 gene (i.e., NR4A3-targeting polynucleotide). Not to be bound by any one theory, in some aspects, by binding to the target sequence within the NR4A3 gene, the polynucleotides of the present disclosure are capable of reducing the level of the NR4A3 gene and/or the encoded protein in a cell (e.g., immune cell).

As described herein, polynucleotides described herein comprise a nucleotide sequence that can specifically bind to a nucleic acid sequence within the NR4A3 gene. Such a nucleotide sequence is also referred to herein as a “binding sequence” or a “guide sequence” or “guide RNA” (gRNA). Accordingly, the term “guide RNA” (gRNA), as used herein, is not particularly limited as long as it can specifically bind to a nucleic acid sequence with the NR4A3 gene, and thereby, reduce the level of the NR4A3 gene and/or NR4A3 protein. Non-limiting examples of such gRNAs are provided throughout the present disclosure (see, e.g., Tables A-D).

In some aspects, the gRNA can be between about 5 and about 100 nucleotides long. In some aspects, the gRNA of a polynucleotide described herein is about 5, about 6, about 7, about 8, about 9, about 10, about 15, about 20, about 25, about 30, about 35, about 40, about 45, about 50, about 60, about 70, about 80, about 90, or about 100 nucleotides in length. In some aspects, the gRNA is between about 10 and about 30 nucleotides in length (e.g., about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides). In some aspects, the gRNA is about 20 nucleotides in length. Additional disclosure relating to gRNAs that are useful for the present disclosure are provided elsewhere in the present application (see, e.g., Section IV.A.1. CRISPR/Cas System).

In some aspects, the gRNA of a polynucleotide described herein is designed to complement or substantially complement a nucleic sequence within the NR4A3 gene (also referred to herein as the “target sequence”). In some aspects, the gRNA can incorporate wobble or degenerate bases to bind multiple sequences (e.g., multiple target sequences within the NR4A3 gene; or a target sequence within the NR4A3 gene and a target sequence within other members of the NR4A family). In some cases, the gRNA can be altered to increase stability. For example, non-natural nucleotides can be incorporated to increase RNA resistance to degradation. In some aspects, the gRNA can be altered or designed to avoid or reduce secondary structure formation in the gRNA. In some aspects, the gRNA can be designed to optimize G-C content. In some aspects, G-C content is between about 40% and about 60% (e.g., about 40%, about 45%, about 50%, about 55%, about 60%). In some aspects, the gRNA can contain modified nucleotides such as, without limitation, methylated or phosphorylated nucleotides. Additional methods of modifying and thereby, improving one or more properties of the polynucleotides described herein are known in the art. Non-limiting examples of such modifications that can be added to a polynucleotide described herein include: a 5′ cap, a 3′ polyadenylated tail, a riboswitch sequence, a stability control sequence, a hairpin, a subcellular localization sequence, a detection or label sequence, a binding site for one or more proteins, a non-natural nucleotide, or combinations thereof. See, e.g., U.S. Publication No. 20210123046A1, which is incorporated herein by reference in its entirety. Additional disclosures relating to such modifications are provided elsewhere in the present disclosure.

As described herein, in some aspects, the nucleic acid molecule comprising the gene editing tool and the nucleic acid molecule comprising the guide RNA can be introduced into a cell as separate nucleic acid molecules (either concurrently or sequentially). In some aspects, the gene editing tool and the guide RNA can be part of a single nucleic acid molecule. For instance, in some aspects, the nucleic acid molecule that comprises a gene editing tool further comprises a guide RNA (e.g., synthethic guide RNA disclosed herein). In some aspects, the nucleic acid molecule that comprises a gene editing tool further comprises a guide RNA (e.g., synthetic guide RNA disclosed herein) and a nucleic acid encoding a Cas nuclease, e.g., a Cas9 nuclease.

Any of the nucleic acids provided herein can be present in whole cells, in a cell lysate, or in a partially purified or substantially pure form.

A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule. Nucleic acids described herein can be obtained using standard molecular biology techniques known in the art.

In some aspects, the present disclosure provides a vector comprising an isolated nucleic acid molecule comprising a gene editing tool for reducing the expression of NR4A3 gene and/or NR4A3 protein (alone or in combination with reduced level of the NR4A1 gene and/or NR4A1 protein and/or the NR4A2 gene and/or NR4A2 protein) in a cell and/or that encode a chimeric antigen receptor or a T cell receptor that can be expressed in the modified cell of the present disclosure.

As described herein, such vectors can be used to modify a cell (e.g., CAR-expressing cells) to express reduced levels of NR4A3 gene and/or NR4A3 protein (alone or in combination with reduced level of the NR4A1 gene and/or NR4A1 protein and/or the NR4A2 gene and/or NR4A2 protein), wherein such modified cells can be used to treat a disease or disorder, such as cancer.

Suitable vectors for the disclosure include expression vectors, viral vectors, and plasmid vectors. In some aspects, the vector is a viral vector.

As used herein, the terms “vector” and “expression vector” refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

The present disclosure contemplates the use of any nucleic acid modification available to the skilled artisan to modify the nucleic acids disclosed herein, e.g., gRNAs and nucleic acids encoding gRNAs, nucleic acids encoding Cas9, vectors comprising nucleic acids encoding at least one gRNA or at least one gRNA and Cas9, nucleic acids encoding a CAR or a TCR, nucleic acids encoding any genome editing tool disclosed herein, nucleic acids encoding an RNAi, or an antisense oligonucleotide.

As used herein, “unmodified” or “natural” nucleosides or nucleobases include the purine bases adenine (A) and guanine (G), and the pyrimidine bases thymine (T), cytosine (C) and uracil (U). In some aspects, a synthetic, modified gRNA comprises at least one nucleoside (“base”) modification or substitution.

The nucleic acids disclosed in the present application (e.g., gRNAs and nucleic acids encoding such gRNAs, as well as nucleic acids encoding Cas9) can comprise one or more modifications. In some aspects, a nucleotide sequence disclosed herein comprises at least one nucleotide analogue. In some aspects, at least one nucleotide analogue introduced by using in vitro translation (IVT) or chemical synthesis is selected from the group consisting of a 2′-O-methoxyethyl-RNA (2′-MOE-RNA) monomer, a 2′-fluoro-DNA monomer, a 2′-O-alkyl-RNA monomer, a 2′-amino-DNA monomer, a locked nucleic acid (LNA) monomer, a cEt monomer, a cMOE monomer, a 5′-Me-LNA monomer, a 2′-(3-hydroxy)propyl-RNA monomer, an arabino nucleic acid (ANA) monomer, a 2′-fluoro-ANA monomer, an anhydrohexitol nucleic acid (HNA) monomer, an intercalating nucleic acid (INA) monomer, and a combination of two or more of said nucleotide analogues. In some aspects, the optimized nucleic acid molecule, e.g., a gRNA, comprises at least one backbone modification, for example, a phosphorothioate internucleotide linkage.

In some aspects, nucleic acids disclosed in the present application (e.g., gRNAs and nucleic acids encoding such gRNAs, as well as nucleic acids encoding Cas9) can be chemically modified at terminal locations, for example by introducing M (2′-O-methyl), MS (2′-O-methyl 3′ phosphorothioate), or MSP (2′-O-methy 3′thioPACE, phosphonoacetate) modifications, or combinations thereof at positions 1, 2, 3 respect to the 5′ and/or 3′ termini. For example, in one aspects, a gRNA of the present disclosure can comprise three M modifications at the three 5′ nucleotides and three M modifications at the three 3′ nucleotides. In some aspects, a gRNA of the present disclosure can comprise three MS modifications at the three 5′ nucleotides and three MS modifications at the three 3′ nucleotides. In some aspects, a gRNA of the present disclosure can comprise three MSP modifications at the three 5′ nucleotides and three MSP modifications at the three 3′ nucleotides.

In some aspects, at least 5%, 10%, 15%, 20%, 25%, 30%, 35%, 40%, 45%, 50%, 55%, 60%, 65%, 70%, 75%, 80%, 85%, 90%, or 95%, or 100% of the uridine, adenosine, guanosine, cytidine nucleosides in an nucleotide sequence disclosed herein, e.g., a gRNA, have been replaced with a nucleoside

In some aspects, a nucleic acid disclosed in the present application (e.g., a gRNA) comprises a nucleotide sequence produced by IVT or chemical synthesis wherein
(i) at least one uridine in the wild type nucleotide sequence has been replaced; and/or,
(ii) at least one adenosine in the wild type nucleotide sequence has been; and/or,
(iii) at least one guanosine in the wild type nucleotide sequence has been replaced; and/or,
(iv) at least one cytidine in the wild type nucleotide sequence has been replaced.

Modified nucleic acids of the present disclosure (e.g., gRNAs) need not be uniformly modified along the entire length of the molecule. Different nucleotide modifications and/or backbone structures can exist at various positions in the nucleic acid. One of ordinary skill in the art will appreciate that the nucleotide analogs or other modification(s) can be located at any position(s) of a nucleic acid such that the function of the nucleic acid is not substantially decreased. A modification can also be a 5′ or 3′ terminal modification. The nucleic acids can contain at a minimum one and at maximum 100% modified nucleotides, or any intervening percentage, such as at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% modified nucleotides.

In some aspects, the nucleic acids provided herein are synthetic, modified DNA molecules encoding RNA (e.g., a gRNA) and/or polypeptides (e.g., Cas9), where the synthetic, modified DNA molecules comprise one or more modifications.

The synthetic, modified nucleic acids described herein (e.g., gRNAs) include modifications to prevent rapid degradation by endo- and exo-nucleases. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

Specific examples of synthetic, modified nucleic acids (e.g., gRNA) compositions useful with the methods described herein include, but are not limited to, modified nucleic acids (e.g., gRNA) containing modified or non-natural internucleoside linkages. Synthetic, modified nucleic acids (e.g., gRNA) having modified internucleoside linkages include, among others, those that do not have a phosphorus atom in the internucleoside linkage. In some aspects, the synthetic, modified nucleic acid (e.g., gRNA) has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, T-5′ linked analogs of these, and those) having inverted polarity wherein the adjacent pairs of nucleoside units are linked 3′-5′ to 5′-3′ or T-5′ to 5′-T. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

Some aspects of the synthetic, modified nucleic acids (e.g., gRNA) described herein include nucleic acids with phosphorothioate internucleoside linkages and oligonucleosides with heteroatom internucleoside linkage, and in particular ˜CH₂—NH—CH₂—, ˜CH₂—N(CH₃)—O—CH₂—[known as a methylene (methylimino) or MMI], ˜CH₂—O—N(CH₃)˜CH₂—, ˜CH₂—N(CH₃)—N(CH₃)—CH₂— and —N(CH₃)˜CH₂—CH₂— [wherein the native phosphodiester internucleoside linkage is represented as —O—P—O—CH₂—] of U.S. Pat. No. 5,489,677, and the amide backbones of U.S. Pat. No. 5,602,240, both of which are herein incorporated by reference in their entirety. In some aspects, the nucleic acid sequences featured herein have morpholino backbone structures of U.S. Pat. No. 5,034,506, herein incorporated by reference in its entirety.

Synthetic, modified nucleic acids (e.g., gRNA) described herein can also contain one or more substituted sugar moieties. The nucleic acids featured herein can include one of the following at the 2′ position: H (deoxyribose); OH (ribose); F; O—, S—, or N— alkyl; O—, S—, or N-alkenyl; O- , S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary modifications include O[(CH₂)nO]mCH₃, O(CH₂)nOCH₃, O(CH₂)nNH₂, O(CH₂)nCH₃, O(CH₂)nONH₂, and O(CH₂)nON[(CH₂)nCH₃)]₂, where n and m are from 1 to about 10. In some aspects, synthetic, modified RNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a reporter group, an intercalator, a group for improving the pharmacokinetic properties of a certain nucleic acid (e.g., a gRNA), or a group for improving the pharmacodynamic properties of a synthetic, modified nucleic acid (e.g., gRNA), and other substituents having similar properties. In some aspects, the modification includes a 2′ methoxyethoxy (2′-O—CH₂CH₂OCH₃, also known as 2′-O-(2-methoxyethyl) or -MOE) (Martin et al, Helv. Chim. Acta, 1995, 78:486-504), i.e., an alkoxy-alkoxy group. Another exemplary modification is 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the nucleic acid sequence, particularly the 3′ position of the sugar on the 3′ terminal nucleotide or in 2′-5′ linked nucleotides and the 5′ position of 5′ terminal nucleotide. A synthetic, modified gRNA can also have sugar mimetics such as cyclobutyl moieties in place of the pentofuranosyl sugar.

As non-limiting examples, synthetic, modified gRNAs described herein can include at least one modified nucleoside including a 2′-O-methyl modified nucleoside, a nucleoside comprising a 5′ phosphorothioate group, a 2′-amino-modified nucleoside, 2′-alkyl-modified nucleoside, morpholino nucleoside, a phosphoramidate or a non-natural base comprising nucleoside, or any combination thereof.

In some aspects, the at least one modified nucleoside is selected from the group consisting of 5-methylcytidine (5mC), N6-methyladenosine (m6A), 3,2′-O-dimethyluridine (m4U), 2-thiouridine (s2U), 2′ fluorouridine, pseudouridine, 2′-O-methyluridine (Um), 2′ deoxyuridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I).

Alternatively, a synthetic, modified gRNA can comprise at least two modified nucleosides, at least 3, at least 4, at least 5, at least 6, at least 7, at least 8, at least 9, at least 10, at least 15, at least 20 or more, up to the entire length of the nucleotide. At a minimum, a synthetic, modified gRNA molecule comprising at least one modified nucleoside comprises a single nucleoside with a modification as described herein. It is not necessary for all positions in a given synthetic, modified gRNA to be uniformly modified, and in fact more than one of the aforementioned modifications can be incorporated in a single synthetic, modified gRNA or even at a single nucleoside within a synthetic, modified gRNA. However, it is preferred, but not absolutely necessary, that each occurrence of a given nucleoside in a molecule is modified (e.g., each cytosine is a modified cytosine e.g., 5mC). However, it is also contemplated that different occurrences of the same nucleoside can be modified in a different way in a given synthetic, modified gRNA molecule (e.g., some cytosines modified as 5mC, others modified as 2′-O-methylcytidine or other cytosine analog). The modifications need not be the same for each of a plurality of modified nucleosides in a synthetic, modified gRNA. Furthermore, in some aspects of the aspects described herein, a synthetic, modified gRNA comprises at least two different modified nucleosides. In some aspects described herein, the at least two different modified nucleosides are 5-methylcytidine and pseudouridine. A synthetic, modified gRNA can also contain a mixture of both modified and unmodified nucleosides.

The gRNA and other nucleic acids disclosed herein, e.g., nucleic acids used for CRISPR gene editing, or a polynucleotide or set of polynucleotides encoding a CAR or a TCR, can be produced using chemical synthesis using an oligonucleotide synthesizer, host cell expression, in vitro translation (IVT), or any other methods known in the art. Naturally occurring nucleosides, non-naturally occurring nucleosides, or combinations thereof, replacing totally or partially naturally occurring nucleosides. Polynucleotide or nucleic acid synthesis reactions can be carried out by enzymatic methods utilizing polymerases. Polymerases catalyze the creation of phosphodiester bonds between nucleotides in a polynucleotide or nucleic acid chain.

Various tools in genetic engineering are based on the enzymatic amplification of a target nucleic acid which acts as a template. For the study of sequences of individual genes or specific regions of interest and other research needs, it is necessary to generate multiple copies of a target nucleic acid from a small sample of polynucleotides or nucleic acids. Such methods can be applied in the manufacture of the gRNAs and other nucleic acids disclosed herein (e.g., RNAi, an ASO, a polynucleotide encoding a Cas, or a polynucleotide or set of polynucleotides encoding a CAR or a TCR).

Polymerase chain reaction (PCR) has wide applications in rapid amplification of a target gene, as well as genome mapping and sequencing. The key components for synthesizing DNA comprise target DNA molecules as a template, primers complementary to the ends of target DNA strands, deoxynucleoside triphosphates (dNTPs) as building blocks, and a DNA polymerase. As PCR progresses through denaturation, annealing and extension steps, the newly produced DNA molecules can act as a template for the next circle of replication, achieving exponentially amplification of the target DNA. PCR requires a cycle of heating and cooling for denaturation and annealing. Variations of the basic PCR include asymmetric PCR (Innis et al., PNAS 85, 9436-9440 (1988)), inverse PCR (Ochman et al., Genetics 120(3), 621-623, (1988)), and reverse transcription PCR (RT-PCR) (Freeman et al., BioTechniques 26(1), 112-22, 124-5 (1999)), the contents of which are incorporated herein by reference in their entirety. In RT-PCR, a single stranded RNA is the desired target and is converted to a double stranded DNA first by reverse transcriptase. Any of the foregoing methods can be utilized in the manufacture of one or more regions of the polynucleotides of the present disclosure (e.g., a gRNA, a polynucleotide encoding a Cas, or a polynucleotide or set of polynucleotides encoding a CAR or a TCR).

Assembling polynucleotides or nucleic acids by a ligase is also widely used. DNA or RNA ligases promote intermolecular ligation of the 5′ and 3′ ends of polynucleotide chains through the formation of a phosphodiester bond. Accordingly, RNA ligases can be used for example to generate a gRNA by 3′ to 5′ intermolecular ligation of a gRNA spacer sequence and a gRNA frame sequence.

Standard methods can be applied to synthesize an isolated polynucleotide sequence encoding an isolated polypeptide of interest. For example, a single DNA or RNA oligomer containing a codon-optimized nucleotide sequence coding for the particular isolated polypeptide can be synthesized. In some aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a gRNA, a polynucleotide encoding a Cas, or a polynucleotide or set of polynucleotides encoding a CAR or a TCR) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. Nos. 8,999,380, 8,710,200, all of which are herein incorporated by reference in their entireties.

### VII. Pharmaceutical Compositions

The present disclosure provides pharmaceutical compositions comprising (a) a cell which has been modified to express reduced levels of NR4A3 gene and/or NR4A3 protein and that have: (i) endogenous expression of NR4A1 and NR4A2 genes and NR4A1 and NR4A2 proteins, (ii) reduced level of NR4A1 gene and/or NR4A1 protein, (iii) reduced level of NR4A2 gene and/or NR4A2 protein, or (iv) reduced level of both the NR4A1 gene and/or NR4A1 protein and NR4A2 gene and/or NR4A2 protein (e.g., such as those cells described herein). The present disclosure provides pharmaceutical compositions comprising (a) a cell which has been modified to express reduced levels of NR4A3 gene and/or NR4A3 protein and a binding molecule and that have: (i) endogenous expression of NR4A1 and NR4A2 genes and NR4A1 and NR4A2 proteins, (ii) reduced level of NR4A1 gene and/or NR4A1 protein, (iii) reduced level of NR4A2 gene and/or NR4A2 protein, or (iv) reduced level of both the NR4A1 gene and/or NR4A1 protein and NR4A2 gene and/or NR4A2 protein (e.g., such as those cells described herein). More specifically, in some aspects, provided herein is a composition comprising a modified cell (e.g., modified immune cell) which: (i) expresses a binding molecule (e.g., CAR or TCR), (ii) has a reduced level of a NR4A3 gene and/or NR4A3 protein, (iii) has an endogenous level of a NR4A1 gene and/or NR4A1 protein, and (iv) has an endogenous level of a NR4A2 gene and/or NR4A2 protein. In some aspects, a composition provided herein comprises a modified cell (e.g., modified immune cell) which: (i) expresses a binding molecule (e.g., CAR or TCR), (ii) has a reduced level of a NR4A3 gene and/or NR4A3 protein, (iii) has a reduced level of a NR4A1 gene and/or NR4A1 protein, and (iv) has an endogenous level of a NR4A2 gene and/or NR4A2 protein. In some aspects, a composition provided herein comprises a modified cell (e.g., modified immune cell) which: (i) expresses a binding molecule (e.g., CAR or TCR), (ii) has a reduced level of a NR4A3 gene and/or NR4A3 protein, (iii) has a reduced level of a NR4A1 gene and/or NR4A1 protein, and (iv) has a reduced level of a NR4A2 gene and/or NR4A2 protein.

In some aspects, for any of the compositions described above, the level of the NR4A3 gene can be reduced using any of the gene editing tools described herein. For instance, in some aspects, any of the gRNAs described herein that can target the NR4A3 gene can be used to reduce the level of the NR4A3 gene (and thereby, reduce the level of the encoded NR4A3 protein).

Accordingly, in some aspects, a composition useful for the present disclosure comprises a cell (e.g., immune cell) which: (i) has been modified with a gRNA that can target the NR4A3 gene, wherein the gRNA comprises, consists of, or consists essentially of the sequence set forth in any one of SEQ ID NOs: 30 and 52-99. In some aspects, a composition useful for the present disclosure comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in any one of SEQ ID NOs: 30 and 52-99. In some aspects, a composition described herein comprises a cell (e.g., immune cell) which: (i) has been modified with a gRNA that can target the NR4A3 gene, wherein the gRNA comprises, consists of, or consists essentially of the sequence set forth in SEQ ID NO: 30. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 52. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 53. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 54. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 55. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 56. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 57. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 58. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 59. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 60. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 61. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 62. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 63. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 64. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 65. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 66. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 67. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 68. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 69. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 70. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 71. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 72. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 73. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 74. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 75. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 76. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 77. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 78. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 79. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 80. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 81. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 82. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 83. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 84. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 85. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 86. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 87. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 88. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 89. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 90. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 91. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 92. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 93. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 94. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 95. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 96. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 97. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 98. In some aspects, a composition described herein comprises a cell (e.g., immune cell) which has been modified with a gRNA that can target the NR4A3 gene, wherein the gRNA comprises, consists of, or consists essentially of the sequence set forth in SEQ ID NO: 99.

In some aspects, a composition useful for the present disclosure comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in any one of SEQ ID NOs: 30 and 52-99. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 30. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 52. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 53. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 54. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 55. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 56. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 57. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 58. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 59. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 60. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 61. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 62. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 63. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 64. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 65. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 66. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 67. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 68. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 69. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 70. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 71. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 72. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 73. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 74. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 75. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 76. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 77. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 78. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 79. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 80. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 81. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 82. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 83. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 84. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 85. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 86. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 87. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 88. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 89. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 90. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 91. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 92. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 93. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 94. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 95. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 96. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 97. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 98. In some aspects, a composition described herein comprises a cell (e.g., immune cell) which: (i) expresses a binding molecule (e.g., CAR or TCR) and (ii) has been modified with a gRNA that can target the NR4A3 gene, wherein the gRNA comprises, consists of, or consists essentially of the sequence set forth in SEQ ID NO: 99.

In some aspects, any of the above-described compositions further comprise a pharmaceutically acceptable carrier, excipient, or stabilizer. As will be apparent to those in the art, unless indicated otherwise, the term “compositions” and “pharmaceutical compositions” can be used interchangeably. Accordingly, any suitable disclosure related to pharmaceutical compositions can equally apply to compositions. Similarly, any suitable disclosure related to compositions can equally apply to pharmaceutical compositions.

As described herein, any of the pharmaceutical compositions described herein can be used to prevent and/or treat a cancer. As described herein, in some aspects, the modified cell present in a pharmaceutical composition disclosed herein is an immune cell, such as a T cell (e.g., CAR or TCR-expressing T cells) or NK cells (e.g., CAR or TCR-expressing NK cells).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutical compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, each of which is herein incorporated by reference in its entirety.

The compositions to be used for in vivo administration can be sterile. This is readily accomplished by filtration through, e.g., sterile filtration membranes.

The present disclosure also provides a cell composition comprising a means for one or more methods described herein, e.g., promoting persistent memory and/or effector function in a population of immune cells. In some aspects, the present disclosure provides a cell composition comprising a means for reducing, ameliorating, or inhibiting exhaustion and/or dysfunction in a population of immune cells. In some aspects, the means comprises modifying an expression of a NR4A3 gene and/or a NR4A3 protein (alone or in combination the NR4A1 gene and/or NR4A1 protein and/or the NR4A2 gene and/or NR4A2 protein) in the population of immune cells. In some aspects, the means comprises modifying an expression of a NR4A3 gene and/or a NR4A3 protein in the population of immune cells.

### VII. Kits

The present disclosure also provides kits for practicing any of the methods of the present disclosure. In some aspects, the disclosure provides a kit comprising (i) a gene editing tool (e.g., comprising a gRNA, which comprises, consists of, or consists essentially of the sequence set forth in any one of SEQ ID NOs: 30 and 52-99) to reduce the expression of NR4A3 gene and/or NR4A3 protein (alone or in combination the NR4A1 gene and/or NR4A1 protein and/or the NR4A2 gene and/or NR4A2 protein), or (ii) a combination thereof, and optionally instructions for treating a tumor according to any of the methods disclosed herein. Also provided is a kit comprising (i) a gene editing tool (e.g., comprising a gRNA, which comprises, consists of, or consists essentially of the sequence set forth in any one of SEQ ID NOs: 30 and 52-99) to reduce the expression of NR4A3 gene and/or NR4A3 protein (alone or in combination the NR4A1 gene and/or NR4A1 protein and/or the NR4A2 gene and/or NR4A2 protein), or (ii) a combination thereof, and optionally instructions for preparing a cell composition according to the methods disclosed herein.

The present disclosure also provides kits for practicing any of the methods of the present disclosure. In some aspects, the disclosure provides a kit comprising (i) a gene editing tool (e.g., comprising a gRNA, which comprises, consists of, or consists essentially of the sequence set forth in any one of SEQ ID NOs: 30 and 52-99) to reduce the expression of NR4A3 gene and/or NR4A3 protein (alone or in combination the NR4A1 gene and/or NR4A1 protein and/or the NR4A2 gene and/or NR4A2 protein), (ii) a vector comprising a ligand binding protein (e.g., chimeric antigen receptor (CAR) (e.g., ROR1 CAR) or a T cell receptor (TCR)), or (iii) a combination thereof, and optionally instructions for treating a tumor according to any of the methods disclosed herein. Also provided is a kit comprising (i) a gene editing tool (e.g., comprising a gRNA, which comprises, consists of, or consists essentially of the sequence set forth in any one of SEQ ID NOs: 30 and 52-99) to reduce the expression of NR4A3 gene and/or NR4A3 protein (alone or in combination the NR4A1 gene and/or NR4A1 protein and/or the NR4A2 gene and/or NR4A2 protein), (ii) a vector comprising a ligand binding protein (e.g., chimeric antigen receptor (CAR) or a T cell receptor (TCR)), or (iii) a combination thereof, and optionally instructions for preparing a cell composition according to the methods disclosed herein.

In some aspects, the present disclosure provides kits comprising the compositions disclosed herein, for example, (i) a cell, e.g. an immune cell, that exhibits reduced expression of NR4A3 gene and/or NR4A3 protein (alone or in combination the NR4A1 gene and/or NR4A1 protein and/or the NR4A2 gene and/or NR4A2 protein), (ii) at least one gRNA targeting the NR4A3 gene, (iii) a nucleic acid encoding at least one gRNA (e.g., a vector), (iv) at least one gRNA and a Cas protein (e.g., Cas9), (v) at least one nucleic acid encoding a gRNA (e.g. a vector) and a Cas protein (e.g., Cas9), (vi) at least one nucleic acid encoding a gRNA (e.g., a first vector) and a nucleic acid encoding a Cas protein such as Cas9 (e.g., a second vector), (vii) a single vector comprising a nucleic acid encoding at least one gRNA and at least one Cas protein, e.g., Cas9, (vii) a vector or set of vector encoding a CAR or a TCR. In some aspects, the kits comprise Cas9 RNPs targeting NR4A3 (e.g., Cas9 RNP including sgRNA GCUCGAGUAGCCCUCCACGA (SEQ ID NO: 30)). Non-limiting examples of other Cas9 RNPs targeting NR4A3 gene, as well as those targeting the NR4A1 and/or NR4A2 gene, are provided elsewhere in the present disclosure (see, e.g., Tables A, C and D). In some aspects, the kits further comprise instructions for their use.

The present disclosure provides kits for the treatment of cancer comprising a modified cell (e.g., immune cell) disclosed herein, wherein the cell exhibits reduced expression of the NR4A3 gene and/or NR4A3 protein (alone or in combination with reduced level of the NR4A1 gene and/or NR4A1 protein and/or the NR4A2 gene and/or NR4A2 protein). The present disclosure provides kits for the treatment of cancer comprising a modified cell (e.g., immune cell) disclosed herein, wherein the cell exhibits reduced expression of the NR4A3 gene and/or NR4A3 protein (alone or in combination with reduced level of the NR4A1 gene and/or NR4A1 protein and/or the NR4A2 gene and/or NR4A2 protein), and wherein the cell expresses a CAR and/or TCR.

In some aspects, the kit comprises at least one gRNA disclosed herein (e.g., comprising a gRNA, which comprises, consists of, or consists essentially of the sequence set forth in any one of SEQ ID NOs: 30 and 52-99), at least one isolated polynucleotide encoding a gRNA disclosed herein, at least one vector encoding a gRNA disclosed herein, a cell comprising at least one vector encoding a gRNA disclosed herein, or a combination thereof. In some aspects, the kit further comprises a Cas9 protein, an isolated polynucleotide encoding a Cas9 protein, or a vector comprising a polynucleotide encoding the Cas9 protein.

The present disclosure further provides a kit or package comprising at least one container means having disposed therein at least one of the above-mentioned gRNAs, Cas9; vectors, cells, or combinations thereof, together with instructions for reducing expression of NR4A3 gene and/or NR4A3 protein (alone or in combination with reduced level of the NR4A1 gene and/or NR4A1 protein and/or the NR4A2 gene and/or NR4A2 protein).

In some aspects, the kit comprises at least one upstream gRNA and a downstream gRNA. Accordingly, in some the kit comprises (i) at least one gRNA comprising a spacer sequence of any one of SEQ ID NOS: 31-38, and (ii) at least one gRNA comprising a spacer sequence of any one of SEQ ID NOS: 41-42.

In a specific aspect, the kit comprises a gRNA comprising a S. pyogenes spacer sequence of SEQ ID NO: 31-38 operably linked to a S. aureus chimeric frame, e.g., the sequence of SEQ ID NO: 39.

In some aspects, each kit comprising a gRNA for a specific bacterial species Cas9 (e.g., S. pyogenes Cas9) further comprises such Cas9.

In some aspects, the kit comprises one or more cells, cultures, or populations of cells expressing a CAR and/or a TCR targeting a cancer antigen.

### EXAMPLES

### Example 1—Guide RNA Screening and Generation of Modified T Cells

To interrogate the role of each NR4A member in countering human T cell exhaustion, ROR1-R12 chimeric antigen receptor (CAR) T cell and NY-ESO-1 T cell receptor (TCR) T cell models were used. CRISPR-Cas9 guide RNAs (gRNAs) were identified that specifically reduced protein expression of each NR4A family member in human T cells transduced with a ROR1 CAR or NY-ESO-1 TCR.

CRISPR-Cas9 guide RNAs for NR4A1 and NR4A2 are shown in Table A. In the experiments described in Examples 2-7, NR4A1 sgRNAs 5 and 6 were used in combination and NR4A2 sgRNAs 1, 2, and 3 were used in combination.

TABLE A
NR4A1 and NR4A2 guide RNAs

| | |
|---|---|
| NR4A1 sgRNA 5 | GAAGUCCUCGAACUUGAAGG |
| (SEQ ID NO: 25) | |
| NR4A1 sgRNA 6 | ACCUUCAUGGACGGCUACAC |
| (SEQ ID NO: 26) | |
| NR4A2 sgRNA 1 | UUGGGAUGGUCAAAGAAGGU |
| (SEQ ID NO: 27) | |
| NR4A2 sgRNA 2 | CAGCCAGGCACUUCUGAAAU |
| (SEQ ID NO: 28) | |
| NR4A2 sgRNA 3 | UCCGGCGACGCUUGUCCACU |
| (SEQ ID NO: 29) | |

Single gRNA specific for NR4A3 were designed and screened to identify gRNA with maximal editing efficiency and maximal protein reduction in three independent experiments (v0, v1, and v2). For all gRNA screening, isolated donor CD4⁺ and CD8⁺ T cells were purchased from AllCells. CD4⁺ and CD8⁺ T cells were thawed and mixed at a 1:1 ratio for activation with 1% (v/v) TransAct (Miltenyi) for 24 hours. The activated T cells were transduced 24 hours later with bi-cistronic (v0) or tri-cistronic lentiviral vectors encoding the anti-ROR1 CAR (v2) or left untransduced (v1). T cells were then electroporated with Cas9 RNPs targeting human NR4A3 utilizing modified guide RNAs (Synthego; v0—Table B, v1—Table C and v2—Table D) and the Lonza 4D Nucleofector unit. Electroporated T cells were transferred into G-Rex culture plates for expansion before cryopreservation in CryoStor media on day 7. One donor was used in Experiments v0 and v1 whereas two independent donors were used in Experiment v2. To evaluate efficiency of protein reduction by flow cytometry at day 7, 3×105 NR4A3-edited or control (electroporated without RNP) T cells were stimulated with CD3/CD28 Dynabeads (in v0 and v1) or PMA+ionomycin (in v2) for two hours in 200 μL of RPMI-1640 (Gibco)+10% fetal bovine serum (Gibco)+1% penicillin/streptomycin in 96 well round bottom plates (Corning) at 37 C to induce maximum NR4A3 expression. After stimulation, cells were stained with surface markers as described above. The cells were then fixed and permeabilized with the FoxP3 Transcription Factor staining buffer kit (eBiosciences) following manufacturer's instructions and intracellular staining was performed using custom fluorochrome conjugated NR4A3 antibody (R&D Systems).

None of the gRNA in v0 reduced NR4A3 protein expression compared to control non-edited cells (Table B). Confirmatory genomic editing efficiency by NGS was not performed. In vi, all 7 gRNA reduced NR4A3 protein expression compared to non-edited control and NGS editing efficiency was performed. Two guides (g4 and g8) had the highest genomic editing (as measured by total percent T cell variance). Despite high editing efficiency, indel characterization of the genomic variants revealed that g8 resulted in a high frequency of undesirable in-frame deletions (Table C). In v2, the top 14 gRNAs were selected based on KO conditions in which NR4A3 protein expression was reduced compared to non-edited controls and/or similar/better to benchmark g4 for both donors (Table D). Selected conditions are further evaluated to confirm genomic editing efficiency by NGS analysis.

TABLE B
NR4A3 guide sequences from Experiment v0 in one donor. NR4A3 protein
was not reduced compared to non-edited controls and editing efficiency
determined by next-generation sequencing (NGS) in bulk T cells on day 7
of production was not performed. N/A = not applicable.

| | | Protein | T cell | |
|---|---|---|---|---|
| | | Reduction | variance | Type of |
| | 5′-3′ sequence | (criteria met) | (%) | Indel |
| g1 (SEQ ID | CAAUAUAGCCCUUCCCCUCC | No | N/A | N/A |
| NO: 48) | | | | |
| g2 (SEQ ID | AACUGGAACCUGGAGGGGAA | No | N/A | N/A |
| NO: 49) | | | | |
| g3 (SEQ ID | UAACUGGAACCUGGAGGGGA | No | N/A | N/A |
| NO: 50) | | | | |

TABLE C
NR4A3 guide sequences from Experiment v1 in one donor. Shown are genomic
editing efficiency by NGS (measured by percent T cell variance) and the indel
characterization (percent of T cell variance) in bulk T cells on day 7 of production.

| | | Protein | | |
|---|---|---|---|---|
| | | Reduction | | |
| | | (criteria | T cell | |
| | 5′-3′ sequence | met) | variance (%) | Type of Indel |
| g4 (SEQ ID | GCUCGAGUAGCCCUCCACGA | Yes | 70.3 | Deletion-42.3% |
| NO: 30) | | | | Insertion-23.2% |
| | | | | Substitution-11.2% |
| g5 (SEQ ID | CCGCUGCAUUUGGUACACGC | Yes | 43.7 | Deletion-20.8% |
| NO: 52) | | | | Insertion-18.9% |
| | | | | Substitution-7.4% |
| g6 (SEQ ID | UGCGGCGCAGACAUACAGCU | Yes | 48.8 | Deletion-17.9% |
| NO: 53) | | | | Insertion-25.9% |
| | | | | Substitution-6.8% |
| g7 (SEQ ID | GCAGCGGCCCUUGAUCAAAG | Yes | 3.6 | Deletion-0.5% |
| NO: 54) | | | | Insertion-3% |
| | | | | Substitution-0% |
| g8 (SEQ ID | AUACAGCUCGGAAUACACCA | Yes | 71.7 | Deletion-58.7% |
| NO: 55) | | | | Insertion-11.5% |
| | | | | Substitution-2.7% |
| g9 (SEQ ID | CCUGCGUGUACCAAAUGCAG | Yes | 36.5 | Deletion-32.9% |
| NO: 56) | | | | Insertion-1.9% |
| | | | | Substitution-2.3% |
| g10 (SEQ ID | GCGGCCCUUGAUCAAAGUGG | Yes | 28 | Deletion-25.9% |
| NO: 57) | | | | Insertion-1.1% |
| | | | | Substitution-1.7% |

TABLE D
NR4A3 guide sequences from Experiment v2 in two donors. Shown are genomic
editing efficiency by NGS (measured by percent T cell variance) and the indel
characterization (percent of T cell variance) in bulk T cells on day 7 of
production. N/A = not applicable; TBD = to be determined.

| | | Protein | T cell | |
|---|---|---|---|---|
| | | Reduction | variance | Type of |
| | 5′-3′ sequence | (criteria met) | (%) | Indel |
| g11 (SEQ ID | GGACUGCUUGAAGUACAUGG | Yes | TBD | TBD |
| NO: 58) | | | | |
| g12 (SEQ ID | CGGGUGGCUCUCAAGCGCGG | No | N/A | N/A |
| NO: 59) | | | | |
| g13 (SEQ ID | GACGACGAGCUCCUGCUGGG | No | N/A | N/A |
| NO: 60) | | | | |
| g14 (SEQ ID | GUCGGGGUUCAUGAUCUCCG | Yes | TBD | TBD |
| NO: 61) | | | | |
| g15 (SEQ ID | GAGGGCUUGAAGUGGAAGAG | No | N/A | N/A |
| NO: 62) | | | | |
| g16 (SEQ ID | GAUGAAGGCGGUCCCCACGG | No | N/A | N/A |
| NO: 63) | | | | |
| g17 (SEQ ID | GAAGGUACUGAUGCUGGGCA | No | N/A | N/A |
| NO: 64) | | | | |
| g18 (SEQ ID | UCCUCCAGCCUCCAGCCCGG | Yes | TBD | TBD |
| NO: 65) | | | | |
| g19 (SEQ ID | AGCAUCAGUACCUUCGUGGA | No | N/A | N/A |
| NO: 66) | | | | |
| g20 (SEQ ID | CGACUACACCAAGCUGACCA | Yes | TBD | TBD |
| NO: 67) | | | | |
| g21 (SEQ ID | UGGUCAGCUUGGUGUAGUCG | Yes | TBD | TBD |
| NO: 68) | | | | |
| g22 (SEQ ID | GCUGGACCCGCCGAUGAAGG | No | N/A | N/A |
| NO: 69) | | | | |
| g23 (SEQ ID | UUGAAGUACAUGGAGGUGCU | Yes | TBD | TBD |
| NO: 70) | | | | |
| g24 (SEQ ID | GUACGGGUGGCUCUCAAGCG | Yes | TBD | TBD |
| NO: 71) | | | | |
| g25 (SEQ ID | CCGCAUAACUGGAACCUGGA | No | N/A | N/A |
| NO: 72) | | | | |
| g26 (SEQ ID | GGGCACGUGUGCCGUGUGCG | No | N/A | N/A |
| NO: 73) | | | | |
| g27 (SEQ ID | UACGGCGUGCGAACCUGCGA | No | N/A | N/A |
| NO: 74) | | | | |
| g28 (SEQ ID | UGGGGACUGCUUGAAGUACA | Yes | TBD | TBD |
| NO: 75) | | | | |
| g29 (SEQ ID | CCUUGGCAGCACUGAGAUCA | Yes | TBD | TBD |
| NO: 76) | | | | |
| g30 (SEQ ID | CCUUGAUCAAAGUGGAGGAG | No | N/A | N/A |
| NO: 77) | | | | |
| g31 (SEQ ID | UGCAUUUGGUACACGCAGGA | No | N/A | N/A |
| NO: 78) | | | | |
| g32 (SEQ ID | UGAUCAAAGUGGAGGAGGGG | No | N/A | N/A |
| NO: 79) | | | | |
| g33 (SEQ ID | GUGGGGACCGCCUUCAUCGG | No | N/A | N/A |
| NO: 80) | | | | |
| g34 (SEQ ID | AGGAGCUCGUCGUCUGGCGA | No | N/A | N/A |
| NO: 81) | | | | |
| g35 (SEQ ID | CCACCUCGGCUACGACCCGA | Yes | TBD | TBD |
| NO: 82) | | | | |
| g36 (SEQ ID | GCGGCGGCGAGGGCUUGAAG | Yes | TBD | TBD |
| NO: 83) | | | | |
| g37 (SEQ ID | CAGCAUCAGUACCUUCGUGG | No | N/A | N/A |
| NO: 84) | | | | |
| g38 (SEQ ID | GCCGAUGAAGGCGGUCCCCA | No | N/A | N/A |
| NO: 85) | | | | |
| g39 (SEQ ID | CCGUCGGGUCGUAGCCGAGG | Yes | TBD | TBD |
| NO: 86) | | | | |
| g40 (SEQ ID | CUACGGCGUGCGAACCUGCG | No | N/A | N/A |
| NO: 87) | | | | |
| g41 (SEQ ID | CCAUAACGCCCCCGCCUGCG | No | N/A | N/A |
| NO: 88) | | | | |
| g42 (SEQ ID | AUAACGCCCCCGCCUGCGGG | No | N/A | N/A |
| NO: 89) | | | | |
| g43 (SEQ ID | GCCGCAUAACUGGAACCUGG | No | N/A | N/A |
| NO: 90) | | | | |
| g44 (SEQ ID | GAAAUCGACAGUACUGACAU | No | N/A | N/A |
| NO: 91) | | | | |
| g45 (SEQ ID | UUUCAGAAGUGUCUCAGUGU | No | N/A | N/A |
| NO: 92) | | | | |
| g46 (SEQ ID | GAAGUGUCUCAGUGUUGGAA | No | N/A | N/A |
| NO: 93) | | | | |
| g47 (SEQ ID | AGUGUUGGAAUGGUAAAAGA | Yes | TBD | TBD |
| NO: 94) | | | | |
| g48 (SEQ ID | GUACAGAUAGUCUGAAAGGG | No | N/A | N/A |
| NO: 95) | | | | |
| g49 (SEQ ID | GUGUUGAGUCUGUUAAAGCU | Yes | TBD | TBD |
| NO: 96) | | | | |
| g50 (SEQ ID | GAUAGUCUGAAAGGGAGGAG | No | N/A | N/A |
| NO: 97) | | | | |
| g51 (SEQ ID | AGUCUGUUAAAGCUCGGACA | No | N/A | N/A |
| NO: 98) | | | | |
| g52 (SEQ ID | GUCCGUACAGAUAGUCUGAA | No | N/A | N/A |
| NO: 99) | | | | |

As described in more detail in the Examples below, NR4A3-edited ROR1 CAR T cells (edited using NR4A3 gRNA g4 SEQ TD NO: 30) displayed the greatest functional benefit, demonstrating significantly prolonged cytotoxicity, cytokine production, T cell persistence, and improved phenotype following continuous ROR1 antigen exposure compared to control ROR1 CAR T cells. NR4A3-edited NY-ESO-1 TCR T cells also displayed the greatest functional benefit, demonstrating prolonged cytotoxicity and cytokine production following continuous NY-ESO-1 antigen exposure compared to control NY-ESO-1 TCR T cells.

### Example 2—Reduced NR4A3 Expression

Reduction of NR4A protein expression was validated in NR4A-edited ROR1 CAR T cells (edited using NR4A3 sgRNA g4, SEQ ID NO: 30) by flow cytometry. Stimulation with CD3/CD28 Dynabeads was used to induce maximum NR4A expression. For flow cytometry analyses, NR4A-edited ROR1 CAR T cells were stained with a live dead dye for 10 minutes at room temperature (RT), blocked with TruStain FcX (Biolegend) for 5 minutes at RT, stained with CCR7 for 15 minutes at 37 C, and then stained with surface marker antibodies for 10 minutes at RT. All the staining was performed in Biolegend cell staining buffer.

3×10⁵ NR4A-edited or control ROR1 CAR T cells were stimulated with CD3/CD28 Dynabeads (Thermo Fisher) at a 3:1 bead-to-cell ratio for two hours in 200 μL of RPMI-1640 (Gibco)+10% fetal bovine serum (Gibco)+1% penicillin/streptomycin in 96 well round bottom plates (Corning) at 37 C to induce maximum NR4A expression. After stimulation, Dynabeads were removed, and cells were stained with surface markers as described above. The cells were then fixed and permeabilized with the FoxP3 Transcription Factor staining buffer kit (eBiosciences) following manufacturer's instructions. Custom fluorochrome conjugated NR4A antibodies (R&D Systems) were used for staining.

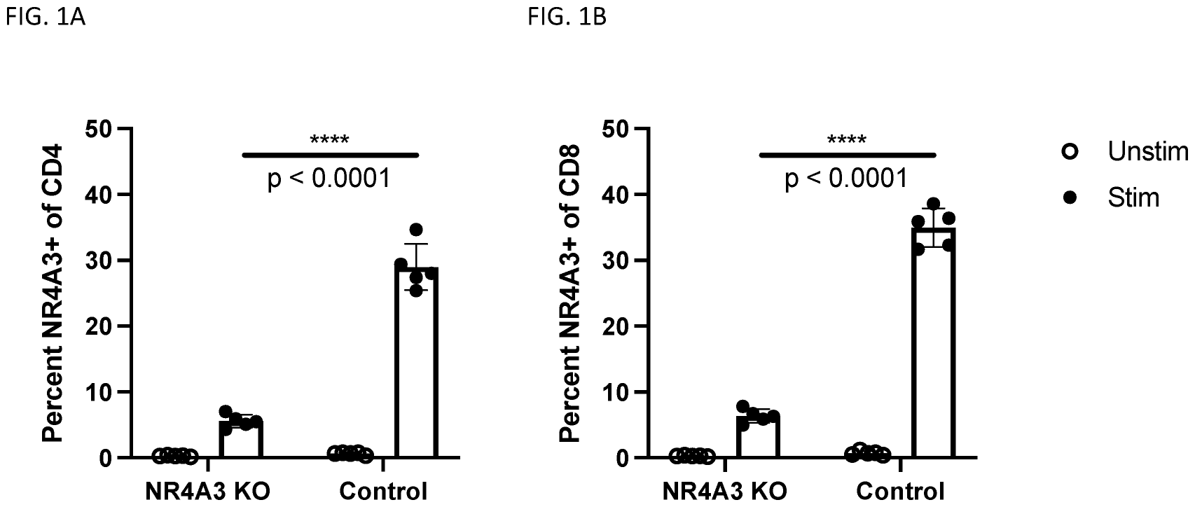

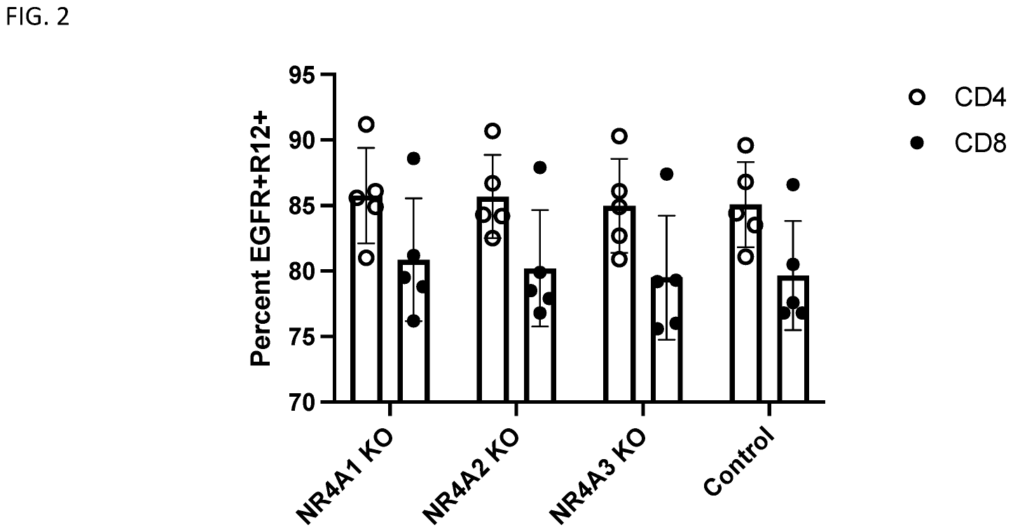

NR4A3 protein expression was significantly reduced in NR4A3-edited ROR1 CD4⁺ and CD8⁺ CAR T cells compared to non-edited controls (FIGS. 1A and 1B). Similarly, high efficiency NR4A1 and NR4A2 protein reduction using CRISPR-Cas9 gRNAs specific for the NR4A1 and NR4A2 genes was achieved (data not shown). Expression of the ROR1 CAR (identified as EGFR⁺R12⁺) was similar across five donors tested and was not affected by NR4A editing (FIG. 2).

### Example 3—Sustained Cytotoxicity and Cytokine Production in Sequential Stimulation

The function of NR4A-edited ROR1 CAR T cells was evaluated in two in vitro exhaustion assays in which CAR T cells are either sequentially or chronically exposed to antigen.

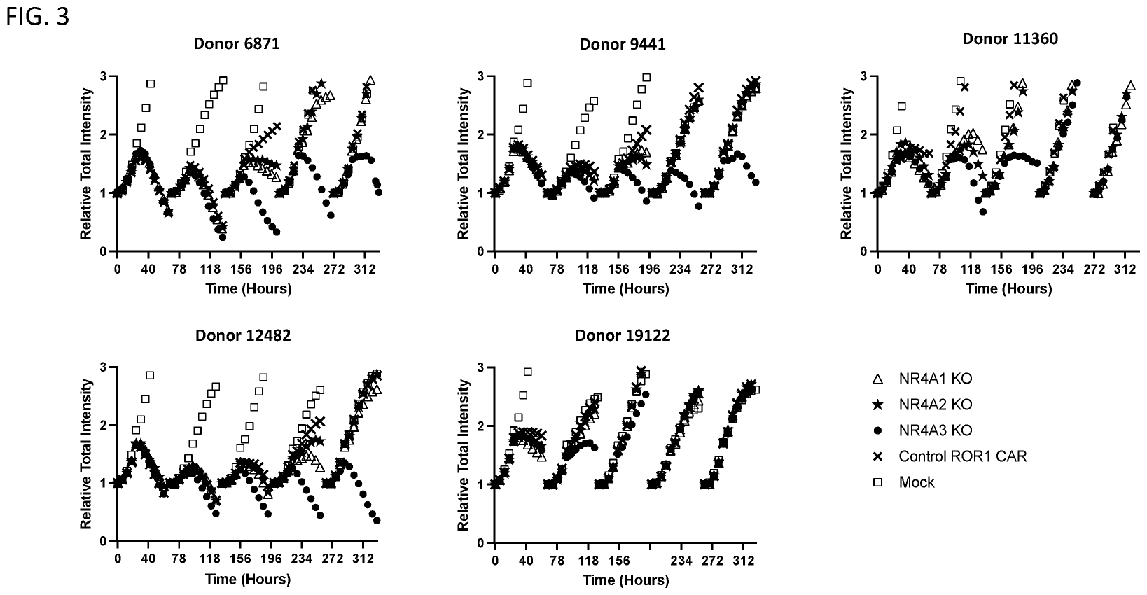

In the sequential stimulation assay, NR4A-edited ROR1 CAR T cells were subjected to five successive stimulations with the H1975 NSCLC ROR1-expressing tumor cell line. In particular, cryopreserved NR4A-edited or control ROR1 CAR T cells were thawed and immediately cultured at a 1:1 E:T ratio with H1975-NLR tumor cells in RPMI-1640 (Gibco)+10% fetal bovine serum (Gibco)+1% penicillin/streptomycin in triplicates in flat 24 well assay plates (Eppendorf). After 3 days of co-culture, wells were resuspended, and 25% of the culture was transferred onto new plates with the same initial number of fresh tumor cells per well. This was repeated for a total of 5 stimulations. Cytotoxicity was measured continuously in the Incucyte during the assay and supernatants were collected 24 hours after setting up each new stimulation to measure cytokine levels. Remaining cells from the triplicate co-culture wells were combined for phenotypic flow analyses as described above. NR4A3-edited ROR1 CAR T cells remained more cytotoxic against H1975 tumor cells, demonstrating a sustained ability to lyse target cells after 3-5 rounds of stimulation compared to NR4A1, NR4A2, or control non-edited ROR1 CAR T cells (FIG. 3).

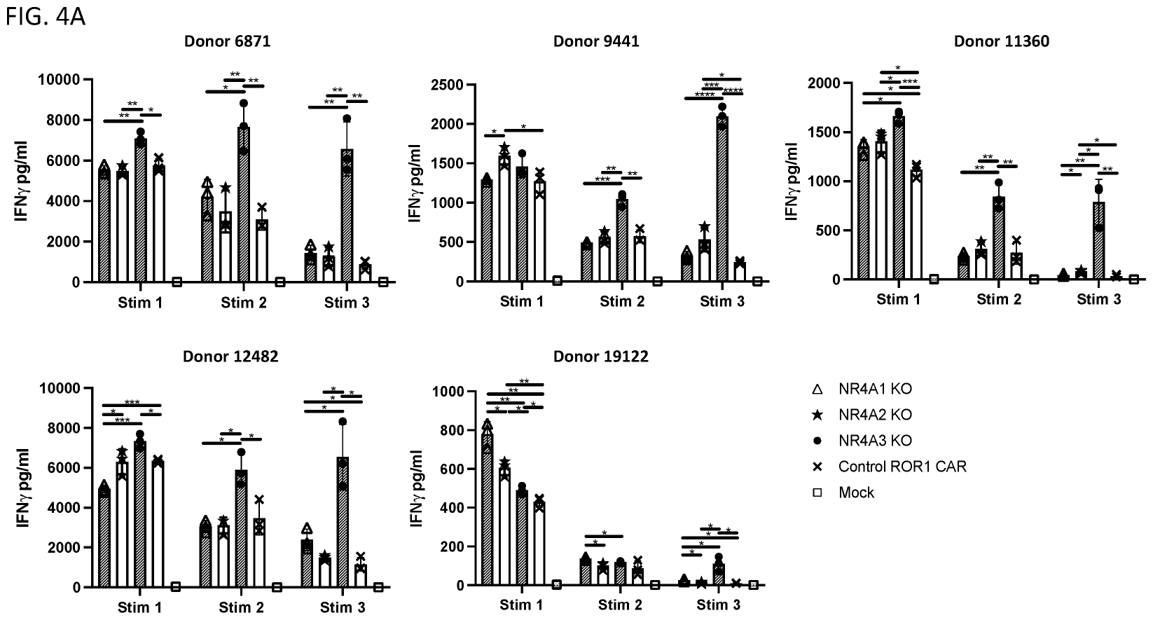

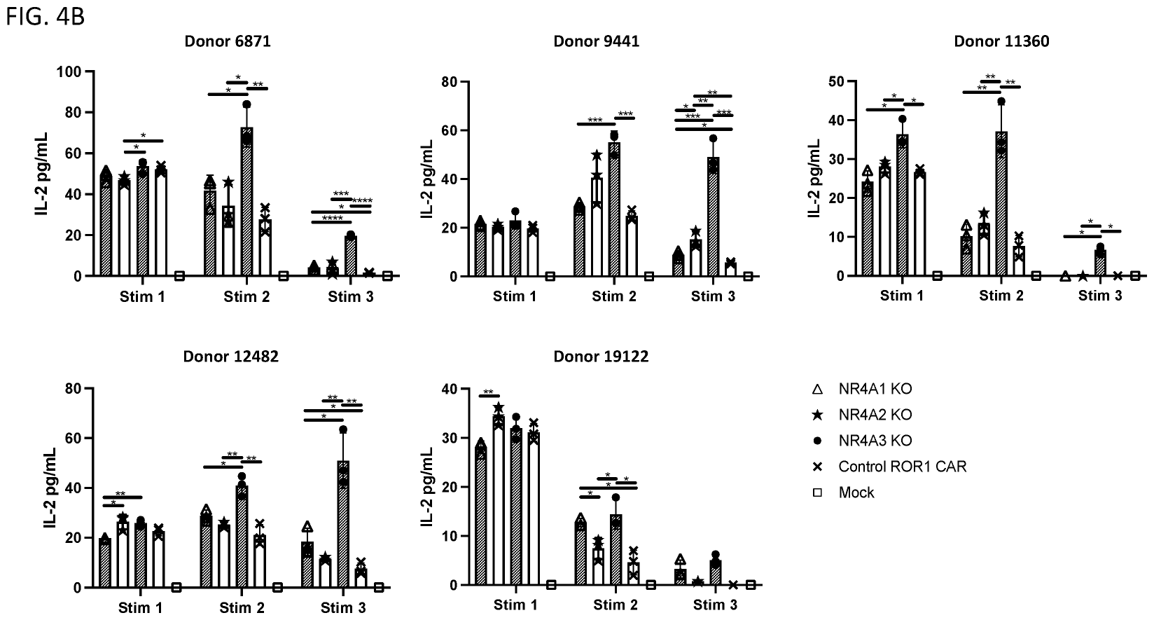

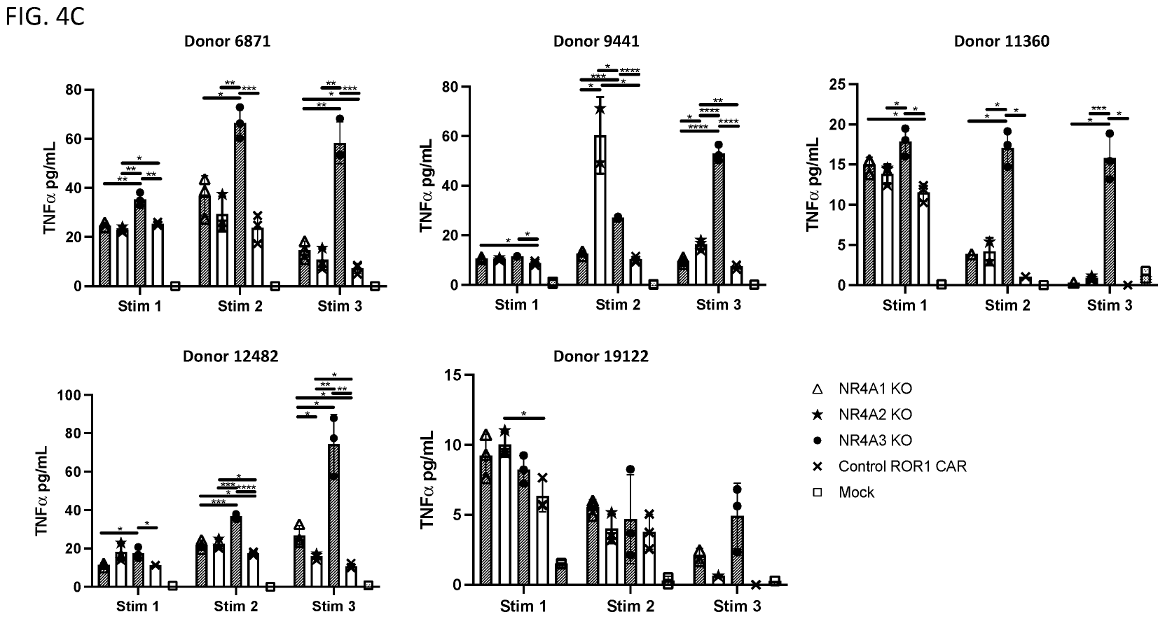

In addition to sustained cytotoxicity, NR4A3-edited ROR1 CAR T cells produced higher levels of IFN-γ, IL-2, and TNF-α compared to NR4A1, NR4A2, or control non-edited ROR1 CAR T cells (FIGS. 4A, 4B, 4C), when stimulated with H1975 NSCLC ROR1-expressing tumor cells. Cytokine levels were measured using Meso Scale Discovery V-Plex proinflammatory panel 1 human kits or custom human IFN-g, IL-2, and TNF-α cytokine kits following the manufacturer's instructions.

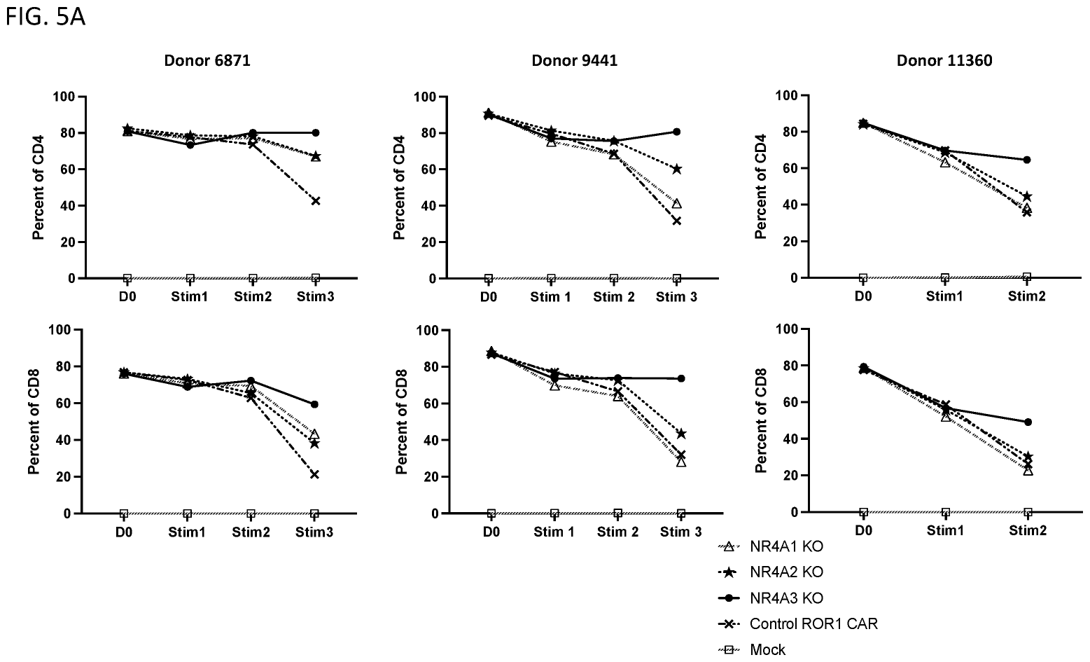

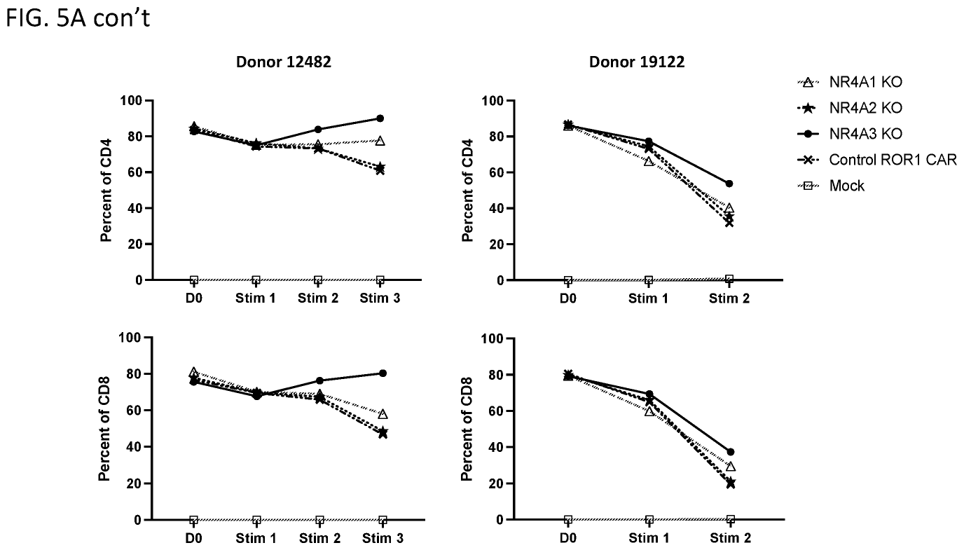

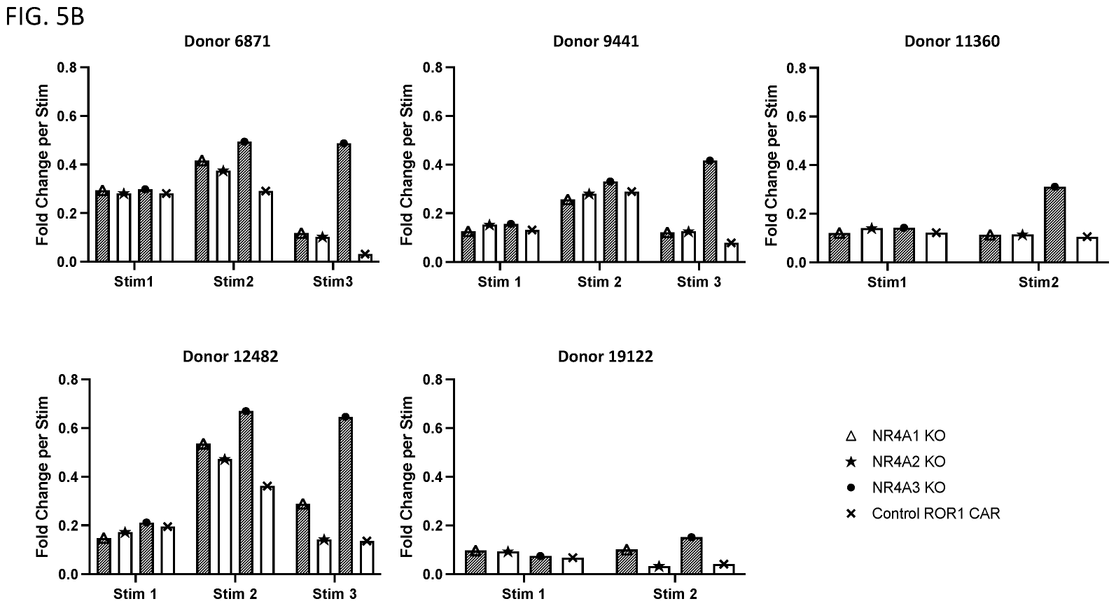

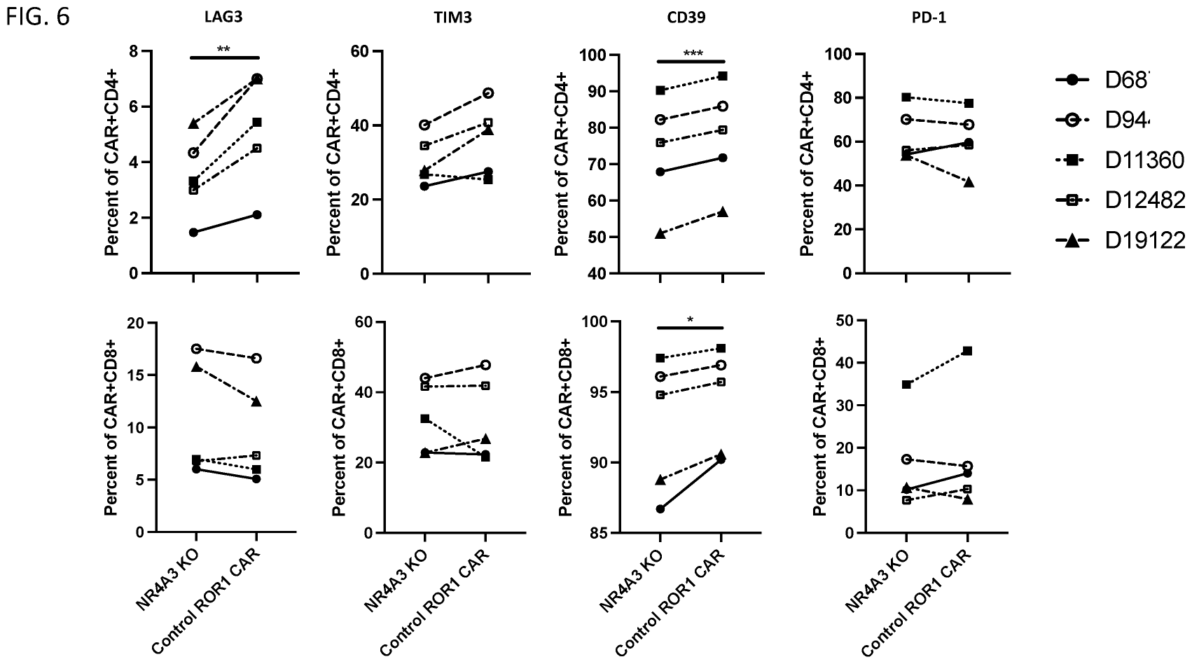

The differences in cytokine production were most notable following later rounds of stimulation, suggesting NR4A3 knockout contributes to sustained functional activity and/or improved CAR T cell survival following prolonged antigen stimulation. Indeed, NR4A3-edited T cells from the sequential stimulation assay maintained a higher frequency of ROR1 CAR-expressing T cells than NR4A1, NR4A2, or control non-edited T cells after several rounds of stimulation (FIG. 5A). Consequently, this correlated to increased persistence of total NR4A3-edited ROR1 CAR T cell numbers at stimulation rounds 2-3 (FIG. 5B). NR4A3-edited ROR1 CAR T cells also had significantly lower expression of LAG3 and CD39 than NR4A1, NR4A2, or control non-edited CAR T cells after the second sequential stimulation against H1975 tumor cells (FIG. 6).

The in vitro sequential stimulation model of T cell exhaustion revealed that NR4A3-edited ROR1 CAR T cells exhibited enhanced and sustained cytotoxicity and cytokine production against ROR1-expressing H1975 tumor cells in five independent donors. The increased functional activity at later rounds of stimulation is likely at least partially due to the increased persistence of NR4A3-edited T cells throughout the assay.

These results were confirmed using a second ROR1⁺ tumor cell line, A549, where NR4A3-edited ROR1 CAR T cells demonstrated sustained cytotoxicity and cytokine production at later stimulations (data not shown). A549-NucLight Red (NLR) and H1975-NLR tumor cells lines were cultured in RPMI-1640 (Gibco)+10% fetal bovine serum (Gibco)+1% penicillin/streptomycin for 2-3 passages before setting up the assays. Cells were trypsinized with TrypLE Express enzyme (Gibco).

### Example 4—Sustained Cytotoxicity and Cytokine Production in Chronic Stimulation

To more directly measure the impact of NR4A3 knockout on cell-intrinsic functional capacity, seven-day chronic stimulation assay was developed to investigate the ability of NR4A-edited ROR1 CAR T cells to counter exhaustion. In this assay, the E:T ratio was re-normalized across all groups at each reset timepoint and before functional assays to remove the impact of differences in CAR T cell numbers on functional readouts.

Cryopreserved NR4A-edited or control ROR1 CAR T cells were thawed and rested for 3 days in TCM, and then cultured at a 1:1 effector-to-target (E:T) ratio with H1975-NLR tumor cells in RPMI-1640 (Gibco)+10% fetal bovine serum (Gibco)+1% penicillin/streptomycin. Surviving CAR T cells were recovered, quantified by flow cytometry for the number of recovered active-Caspase3⁻CD3⁺EGFR⁺R12+ cells, and replated at 1:1 E:T ratio with fresh tumor cells every 2-3 days for a total of seven days. NR4A-edited ROR1 CAR T cells were stained with a live dead dye for 10 minutes at room temperature (RT), blocked with TruStain FcX (Biolegend) for 5 minutes at RT, stained with CCR7 for 15 minutes at 37 C, and then stained with surface marker antibodies for 10 minutes at RT. The NR4A-edited ROR1 CAR T cells were further fixed and permeabilized with the BD Cytofix/CytoPerm kit following manufacturer's instructions and stained with active-Caspase3. All the staining was performed in Biolegend cell staining buffer.

Functional cytotoxicity and cytokine production was assessed on day 0 when the chronic stimulation was initiated and on day 7 at the end of chronic stimulation. Cytotoxicity was measured using the Incucyte in flat 96 well assay plates (Eppendorf) for 72 hours at a 1:1 E:T ratio for A549-NLR tumor cells and a 1:5 E:T for H1975-NLR tumor cells. Supernatants were collected 24 hours after setting up the Incucyte assay plates to measure cytokine levels.

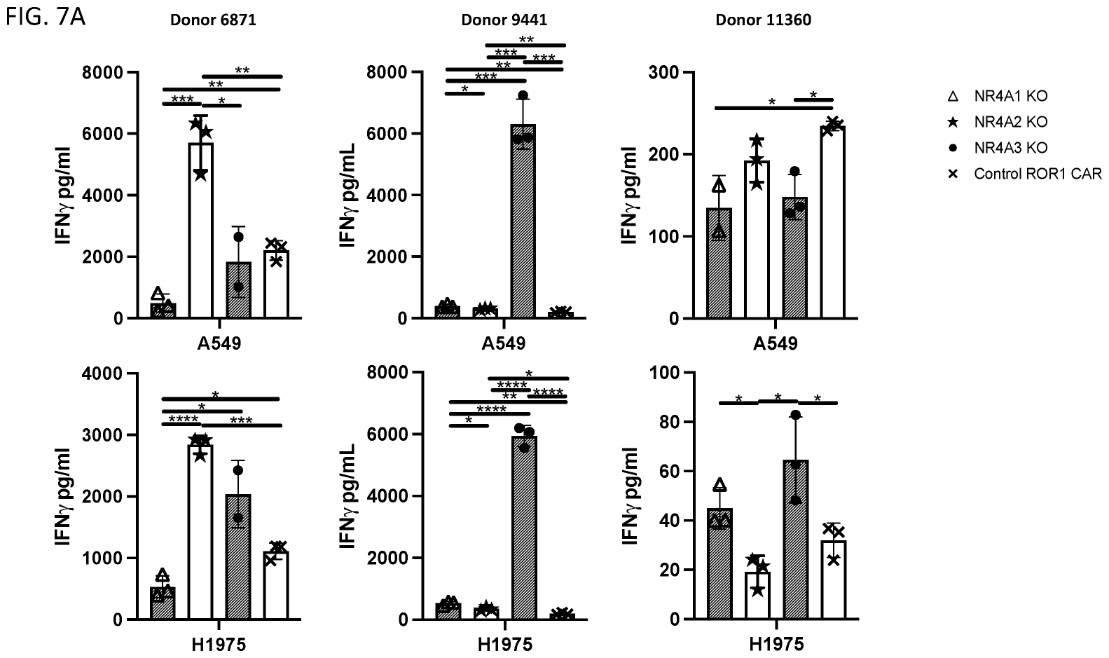

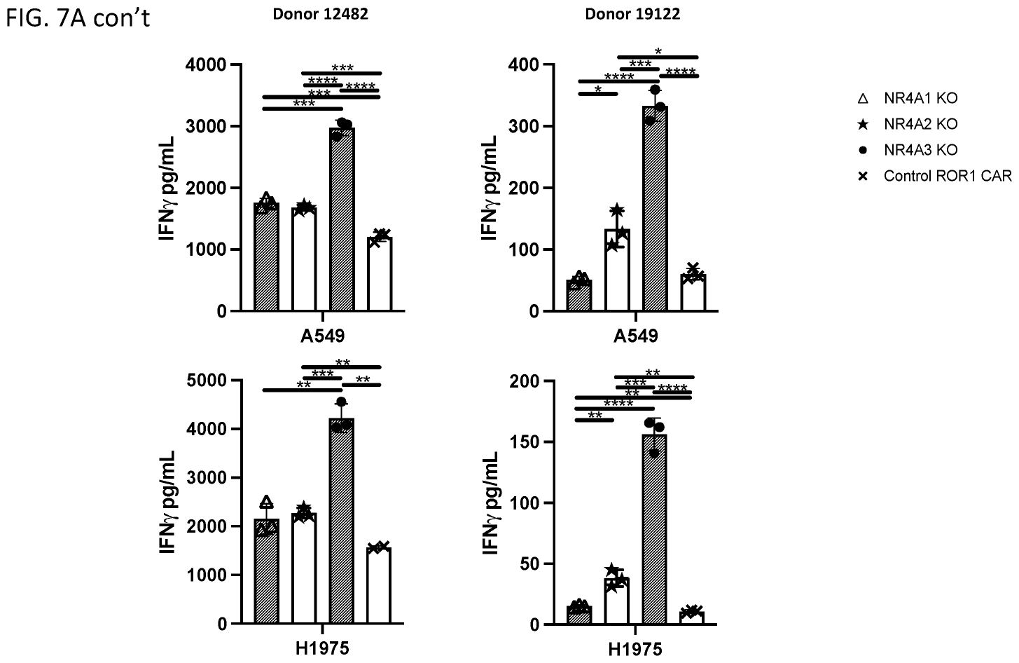

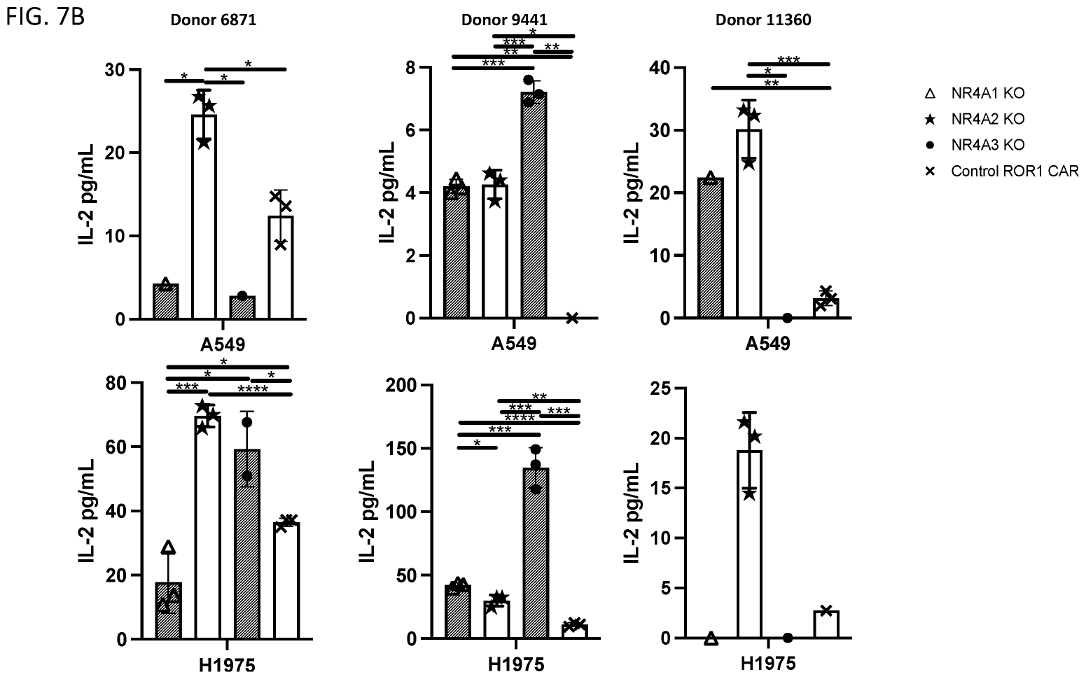

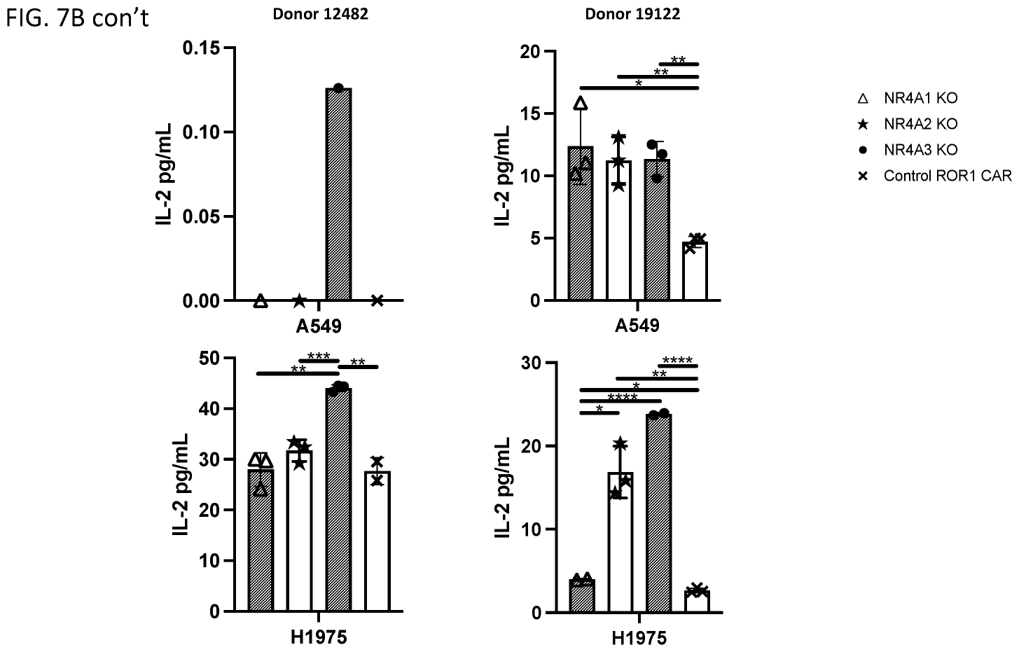

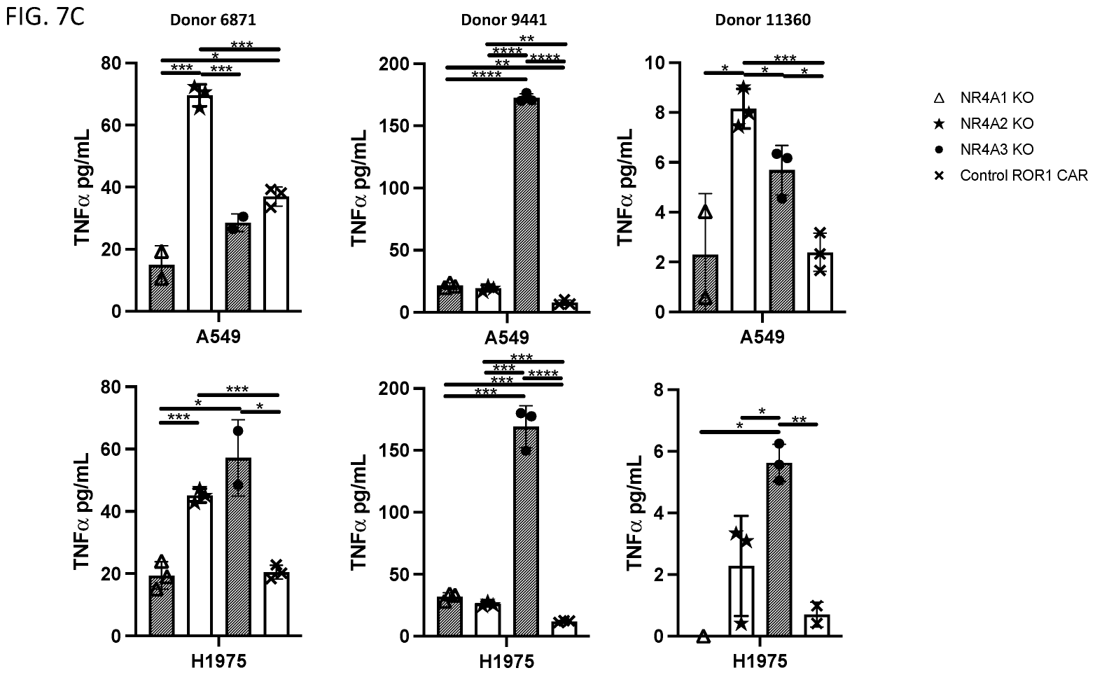

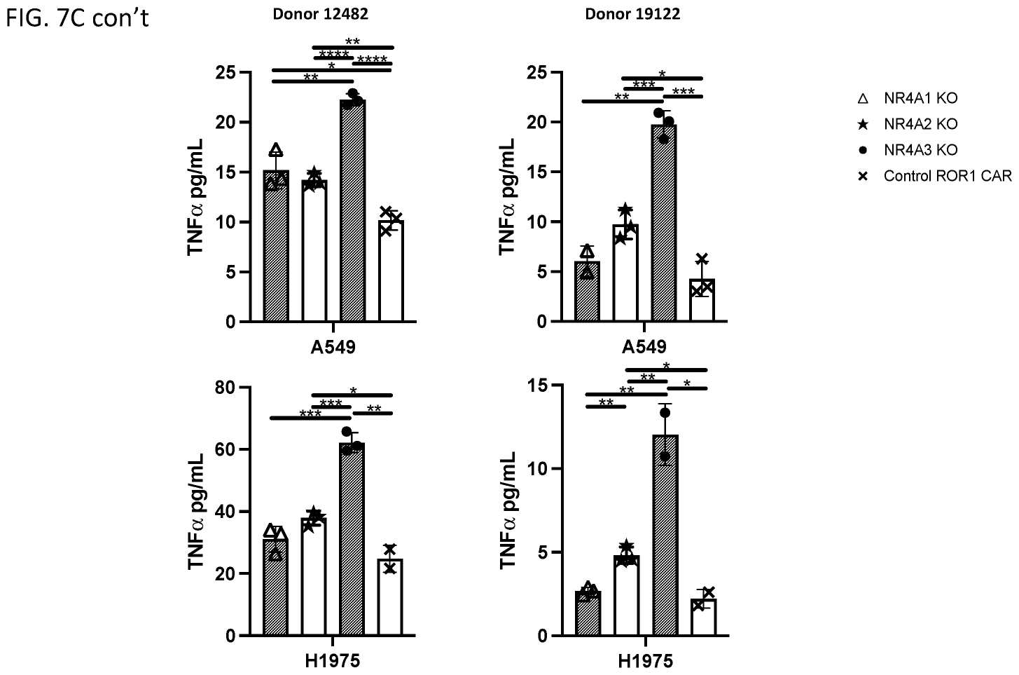

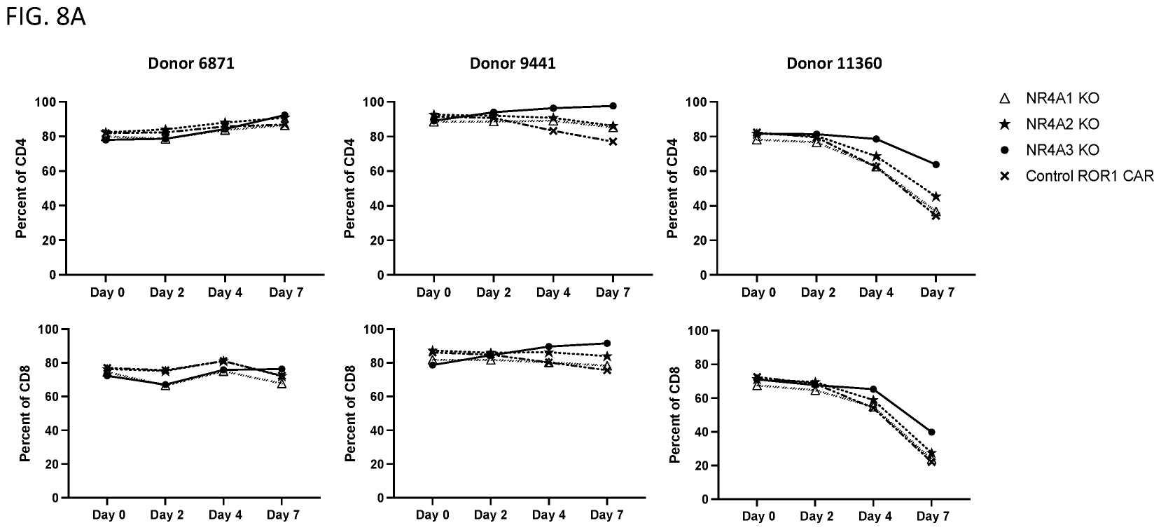

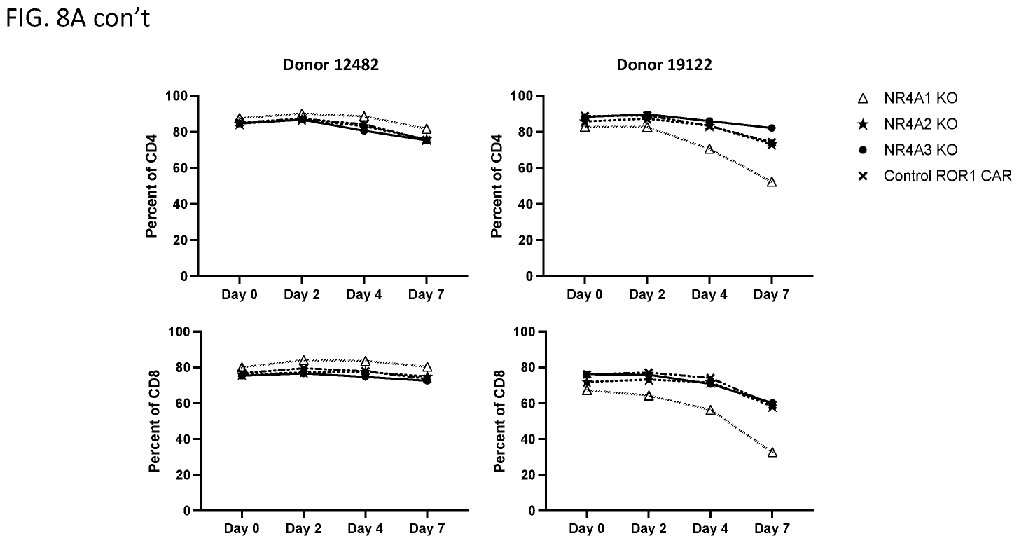

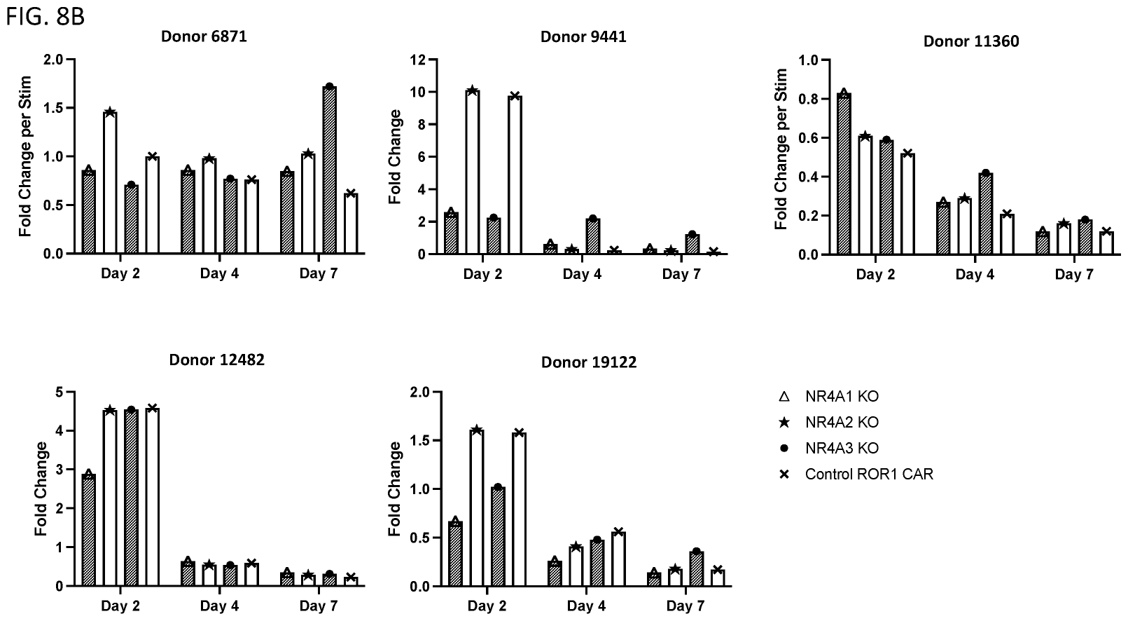

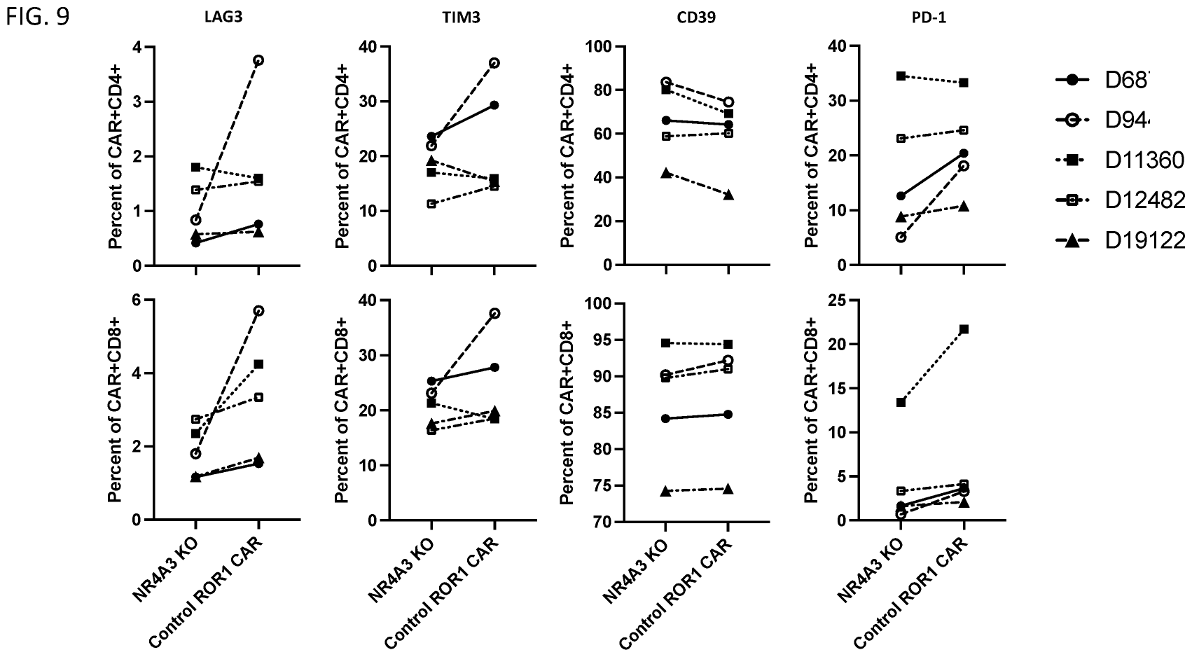

Similar to the sequential stimulation assay, NR4A3-edited ROR1 CAR T cells produced higher levels of IFN-γ, IL-2, and TNF-α after seven days of chronic ROR1 antigen exposure from H1975 tumor cells compared to NR4A1, NR4A2, or control non-edited CAR T cells (FIG. 7). Similar findings were observed in a seven-day chronic stimulation assay using A549 tumor cells where NR4A3-edited ROR1 CAR T cells maintained increased cytokine production compared to NR4A1, NR4A2, or control non-edited CAR T cells (data not shown). A small increase in the percentage of EGFR⁺R12⁺ CAR-expressing cells was observed among NR4A3-edited ROR1 CAR T cells compared to other CAR T cells during H1975 chronic stimulation (FIG. 8A) and total NR4A3-edited ROR1 CAR T cell numbers were slightly improved in most donors on day 7 (FIG. 8B). Furthermore, NR4A3-edited ROR1 CAR T cells tended to have lower inhibitory marker expressions than control non-edited ROR1 CAR T cells after seven days of chronic ROR1 antigen exposure (not statistically significant) (FIG. 9).

### Example 5—Antitumor Efficacy

Lastly, an in vivo H1975 xenograft model was used to determine whether the phenotype and improved function of NR4A3-edited ROR1 CAR T cells from the in vitro assays are recapitulated.

Parental H1975 tumor cells were cultured in RPMI-1640 (Gibco)+10% fetal bovine serum (Gibco) for three passages before implantation into 6-8-week-old NSG HLA double-knockout mice (Jackson Labs). Cells were trypsinized with TrypLE Select enzyme, resuspended in HBSS (Gibco), and mixed with Matrigel (Corning) at a 1:1 ratio. Five million H1975 tumor cells were implanted into the flank of each mouse. T cells were adoptively transferred into randomized tumor-bearing mice when tumors reached 80-120 mm³ in size.

Tumor volumes and body weight were measured twice weekly until the endpoint when tumors reached >2000 mm³, >20% body weight loss, ulceration, labored breathing, severely restricted mobility or inability to upright, or 60 days after T cell transfer.

To prepare T cells for adoptive transfer, cryopreserved NR4A-edited or control ROR1 CAR T cells were thawed and washed with RPMI-1640 (Gibco)+25 mM HEPES (Gibco) prior to adoptive transfer into tumor-bearing mice. Mice were injected i. v. via the tail vein with 100 μL of 0.6 million (low dose) or 2 million (high dose) active-Caspase3⁻CD3⁺EGFR⁺R12⁺ T cells. n=5 mice per treatment group.

GraphPad Prism unpaired t-test, paired t-test, and log-rank (Mantel-Cox) test were used for statistical analysis. *p<0.05, ** p<0.005, * * * p<0.001, **** p<0.0001.

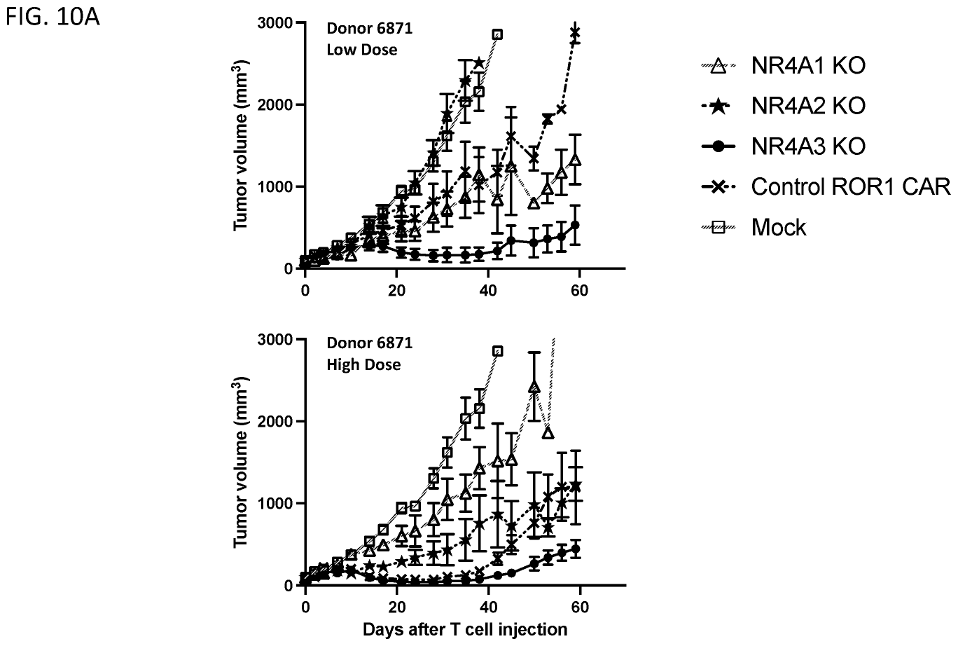

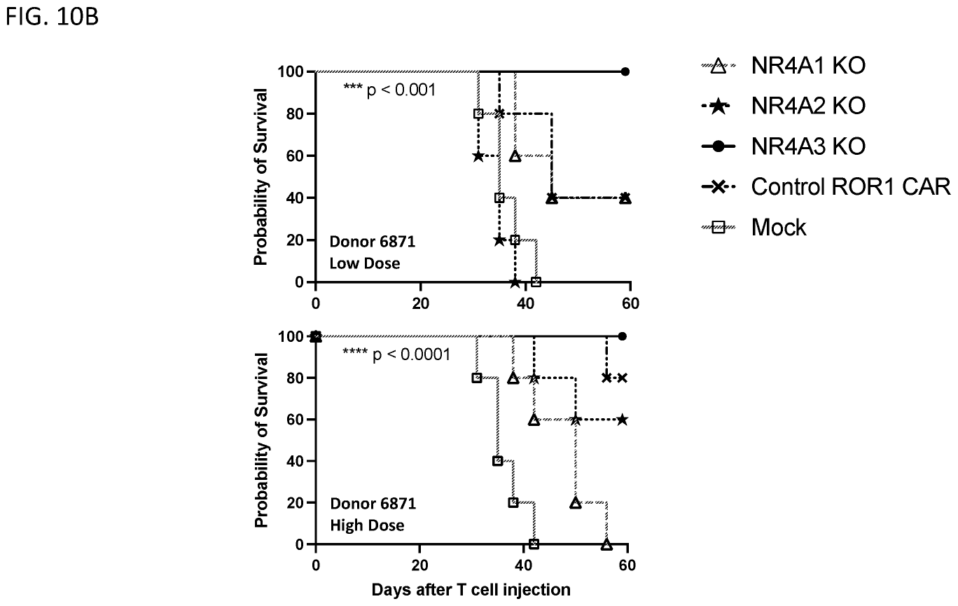

NR4A3-edited ROR1 CAR T cells showed potent and improved anti-tumor efficacy at two different dose levels of ROR1 CAR T cells (FIG. 10A). Moreover, mice adoptively transferred with NR4A3-edited ROR1 CAR T cells had increased survival sixty days post T cell injection compared to other T cell groups (FIG. 10B).

NR4A3-edited ROR1 CAR T cells exhibited robust cytotoxicity and cytokine production, better maintenance and persistence of ROR1 CAR T cells, and displayed a phenotype that trended towards reduced exhaustion, as demonstrated by the two in vitro assays (e.g., sequential and chronic stimulation) and the in vivo H1975 xenograft model. Therefore, editing NR4A3 in the context of ROR1-R12 CAR T cells can improve cellular immunotherapy against ROR1-expressing solid tumors.

### Example 6—Sustained Cytotoxicity and Cytokine Production in Sequential Stimulation

To further assess the effect that reduced NR4A level has on CAR T cell function (particularly in the context of chronic antigen stimulation), anti-ROR1 CAR T cells with reduced level of multiple members of the NR4A family were generated using the methods and sgRNA provided in Example 1. Specifically, anti-ROR1 CAR T cells having reduced levels of the following NR4A family members were produced: (1) both NR4A1 and NR4A2 (NR4A 1+2 Double Knockout), (2) both NR4A1 and NR4A3 (NR4A 1+3 Double Knockout), (3) both NR4A2 and NR4A3 (NR4A 2+3 Double Knockout), and (4) NR4A1, NR4A2, and NR4A3 (NR4A Triple Knockout). Non-NR4A-edited anti-ROR1 CAR T cells and anti-ROR1 CAR T cells having reduced level of NR4A1 alone, NR4A2 alone, and NR4A3 alone (see Examples 1 and 2) were also used for comparison purposes. The different anti-ROR1 CAR T cells were stimulated with antigen (either the A549-NLR or H1975-NLR cells) in a sequential stimulation assay as described in Example 3. Cytotoxicity was measured continuously in the Incucyte during the assay and supernatants were collected 24 hours after setting up each new stimulation to measure cytokine levels.

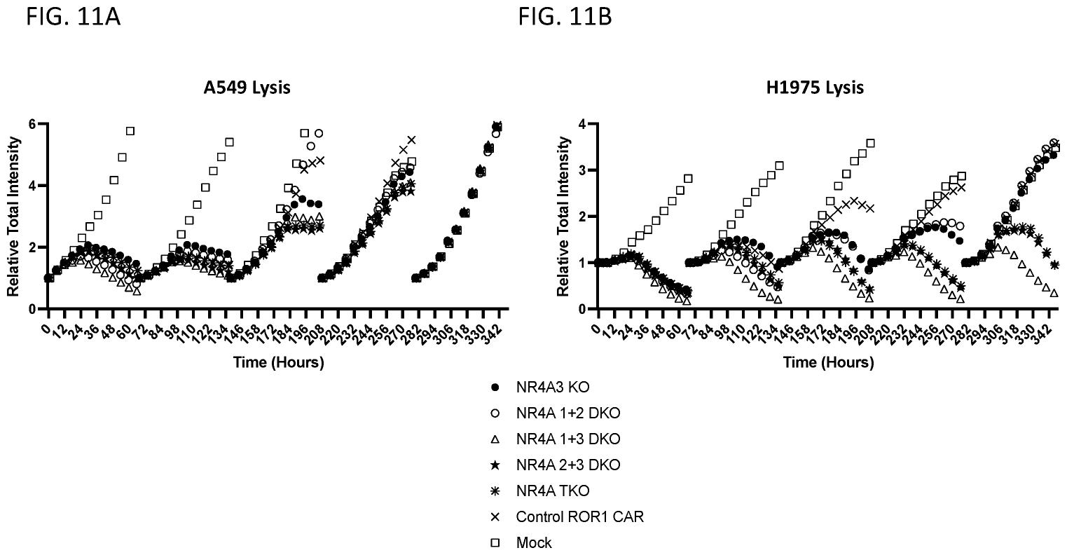

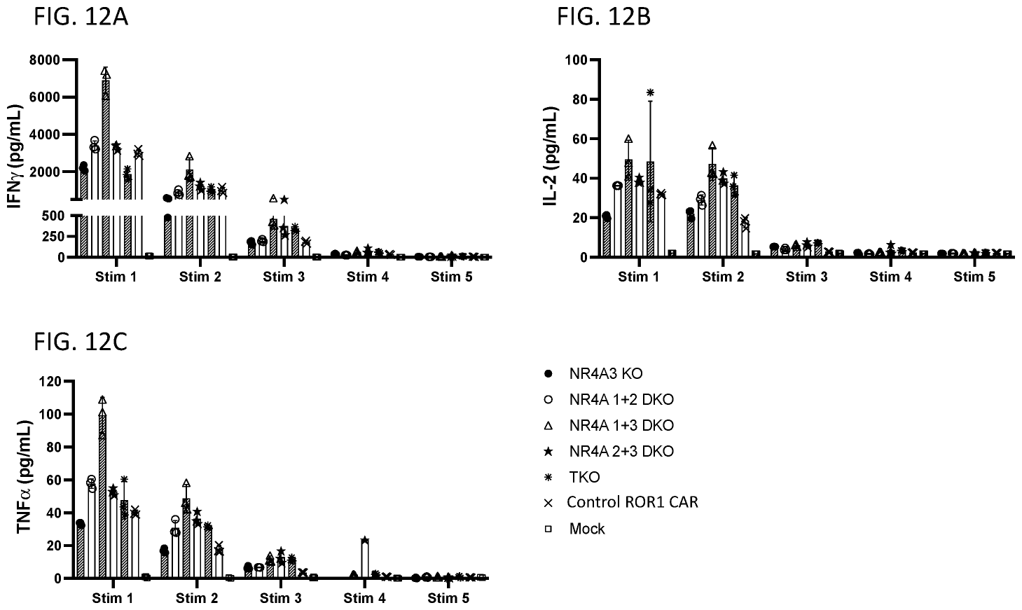

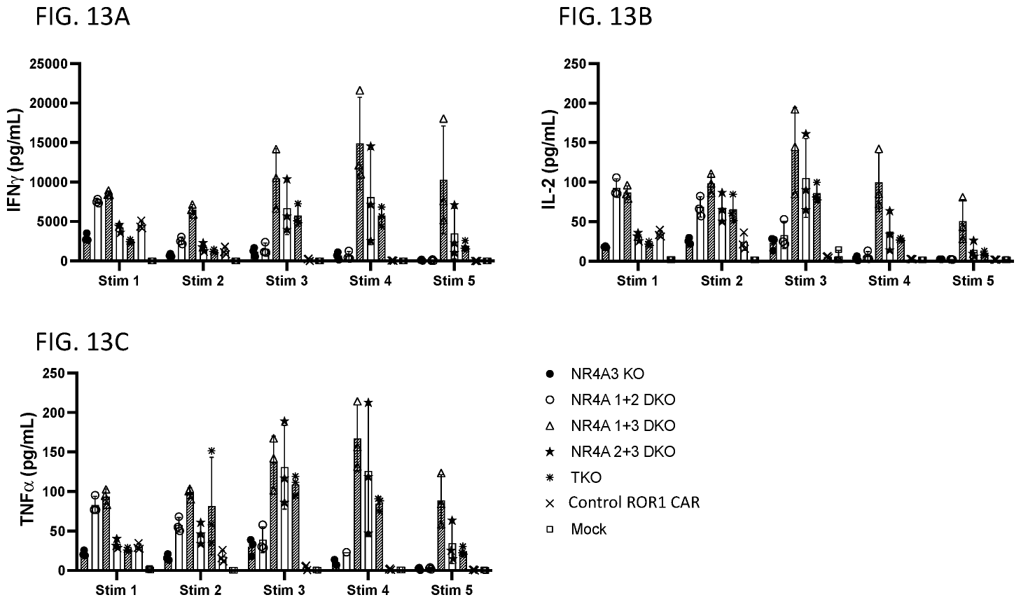

As shown in FIGS. 11A and 11B, even after repeated antigen stimulation, the different anti-ROR1 NR4A double and triple knockout CAR T cells were able to maintain their ability to effectively lyse the ROR1⁺ tumor cells compared to the mock anti-ROR1 CAR T cells. In addition to sustained cytotoxicity, the NR4A double and triple KO anti-ROR1 CAR T cells also maintained their ability to produce different cytokines (IFN-γ, IL-2, and TNF-α) after repeated antigen stimulation. In particular, KO combinations containing NR4A3 KO (NR4A1/NR4A3 DKO, NR4A2/NR4A3 DKO and TKO) showed the largest impact, confirming the importance of NR4A3 KO in the enhanced activity of the modified cells.

These results further demonstrate the therapeutic benefits of reducing NR4A levels in immune cells (e.g., CAR T cells), particularly in the context of chronic antigen stimulation.

### Example 7—Sustained Cytotoxicity and Cytokine Production of Engineered TCR T Cells in Sequential Stimulation

The function of single NR4A-edited NY-ESO-1 TCR T cells (in which TCR T cells were edited at either NR4A1, NR4A2, or NR4A3) were evaluated in an in vitro exhaustion assay in which TCR T cells are sequentially exposed to antigen. Before setting up the assays, A375-NucLight Red (NLR) tumor cells lines were cultured in RPMI-1640 (Gibco)+10% fetal bovine serum (Gibco)+1% penicillin/streptomycin for 2-3 passages. Cells were trypsinized with Accutase enzyme (StemCell Technologies).

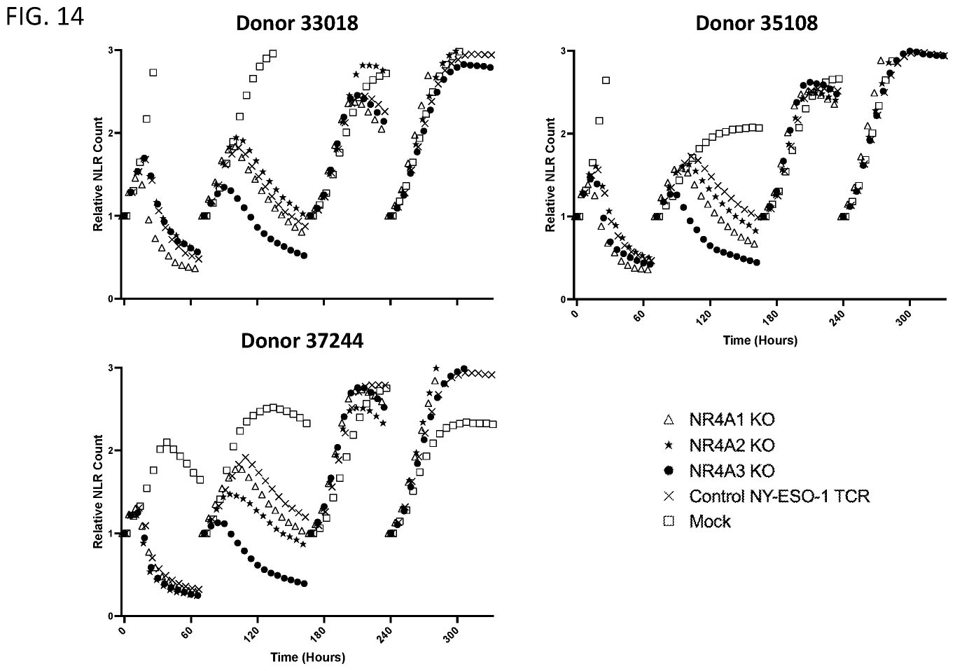

In the sequential stimulation assay, control and NR4A-edited NY-ESO-1 TCR T cells were subjected to four successive stimulations with the A375 melanoma NY-ESO-1/LAGE-la-expressing tumor cell line. In particular, cryopreserved NY-ESO-1 TCR T cells were thawed and immediately cultured at a 1:1 E:T ratio of cParp⁻CD3⁺ TCRvβ13.1⁺ TCR T cells with A375-NLR tumor cells in RPMI-1640 (Gibco)+10% fetal bovine serum (Gibco)+1% penicillin/streptomycin in triplicates in flat 96 well assay plates (Eppendorf). After 3 or 4 days of co-culture, wells were resuspended, and 25% of the culture was transferred onto new plates with the same initial number of fresh tumor cells per well. This was repeated for a total of 4 stimulations. Cytotoxicity was measured continuously in the Incucyte during the assay and supernatants were collected 24 hours after setting up each new stimulation to measure cytokine levels. Similar to the ROR1-CAR-T cell setting, NR4A3 KO NY-ESO-1 TCR T cells remained the most cytotoxic against A375 tumor cells, demonstrating a more sustained ability to lyse target cells after 2 or 3 rounds of stimulation compared to controls in 3 different donors (FIG. 14).

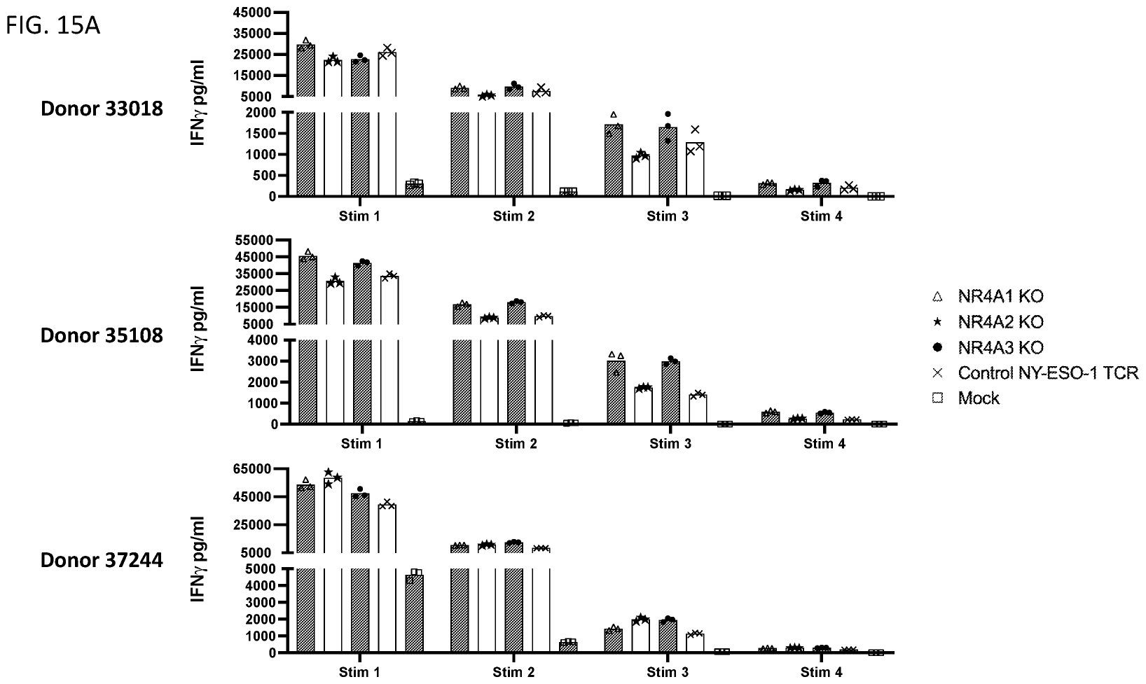

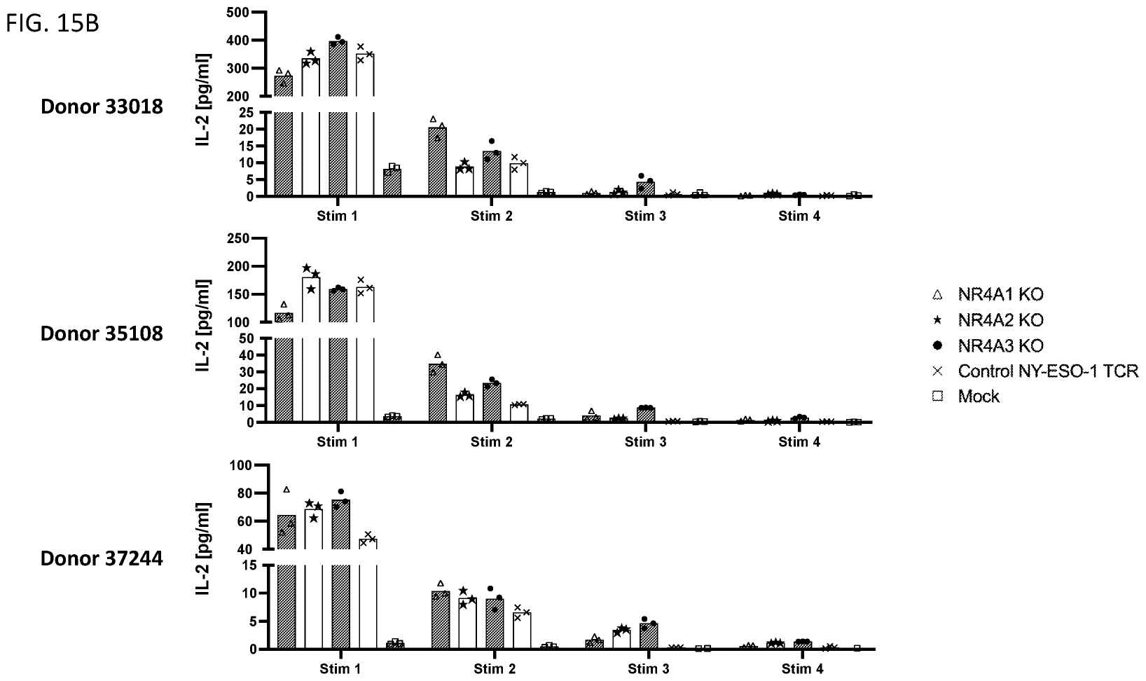

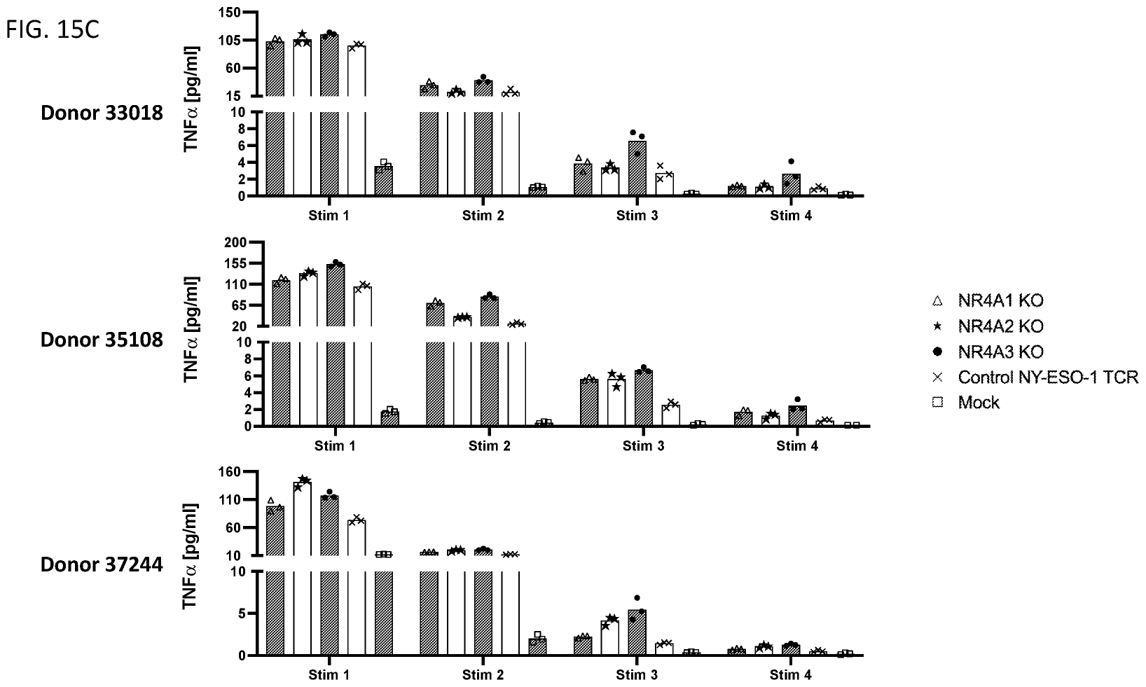

In addition to sustained cytotoxicity, NR4A3 KO NY-ESO-1 TCR T cells also produced higher IFN-γ, IL-2, and TNF-α compared to non-edited control NY-ESO-1 TCR T cells (FIG. 15 and Tables 3-5) when stimulated with A375 melanoma NY-ESO-1/LAGE-la-expressing tumor cells in most donors at most timepoints measured. Similar results were observed when T cells were serially stimulated using a second NY-ESO-1/LAGE-la-expressing tumor cell line H1703 (data not shown). Cytokine levels were measured using Meso Scale Discovery V-Plex proinflammatory panel 1 human kits or custom human IFN-γ, IL-2, and TNF-α cytokine kits following the manufacturer's instructions. The differences in cytokine production were most notable following later rounds of stimulation.

TABLE 3
Unpaired t-test statistical analysis of secreted interferon-gamma (IFN-γ) produced from
NR4A-edited and control non-edited NY-ESO-1 TCR T cells during the
A375 sequential stimulation assay corresponding to FIG. 15.

| D33018 | | | | | |
|---|---|---|---|---|---|
| IFN-γ | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ** | X | X | X | X |
| KO | | | | | |
| NR4A3 | ** | ns | X | X | X |
| KO | | | | | |
| Control | ns | ns | ns | X | X |
| TCR | | | | | |
| Mock | **** | **** | **** | **** | X |
| D33018 | | | | | |
| IFN-γ | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ** | X | X | X | X |
| KO | | | | | |
| NR4A3 | ns | ** | X | X | X |
| KO | | | | | |
| Control | ns | ns | ns | X | X |
| TCR | | | | | |
| Mock | ** | **** | *** | ** | X |
| D33018 | | | | | |
| IFN-γ | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ** | X | X | X | X |
| KO | | | | | |
| NR4A3 | ns | * | X | X | X |
| KO | | | | | |
| Control | ns | ns | ns | X | X |
| TCR | | | | | |
| Mock | *** | **** | *** | ** | X |
| D33018 | | | | | |
| IFN-γ | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ** | X | X | X | X |
| KO | | | | | |
| NR4A3 | ns | * | X | X | X |
| KO | | | | | |
| Control | * | ns | ns | X | X |
| TCR | | | | | |
| Mock | **** | **** | ** | ** | X |
| D35108 | | | | | |
| IFN-γ | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ** | X | X | X | X |
| KO | | | | | |
| NR4A3 | ns | ** | X | X | X |
| KO | | | | | |
| Control | ** | ns | ** | X | X |
| TCR | | | | | |
| Mock | **** | **** | **** | **** | X |
| D35108 | | | | | |
| IFN-γ | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | *** | X | X | X | X |
| KO | | | | | |
| NR4A3 | ns | **** | X | X | X |
| KO | | | | | |
| Control | *** | ns | *** | X | X |
| TCR | | | | | |
| Mock | **** | **** | **** | **** | X |
| D35108 | | | | | |
| IFN-γ | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ** | X | X | X | X |
| KO | | | | | |
| NR4A3 | ns | *** | X | X | X |
| KO | | | | | |
| Control | ** | ** | **** | X | X |
| TCR | | | | | |
| Mock | *** | **** | **** | **** | X |
| D35108 | | | | | |
| IFN-γ | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | *** | X | X | X | X |
| KO | | | | | |
| NR4A3 | ns | *** | X | X | X |
| KO | | | | | |
| Control | *** | * | *** | X | X |
| TCR | | | | | |
| Mock | **** | **** | **** | **** | X |
| D37244 | | | | | |
| IFN-γ | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | * | X | X | X |
| KO | | | | | |
| Control | ** | ** | * | X | X |
| TCR | | | | | |
| Mock | **** | **** | **** | **** | X |
| D37244 | | | | | |
| IFN-γ | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ns | X | X | X | X |
| KO | | | | | |
| NR4A3 | ** | * | X | X | X |
| KO | | | | | |
| Control | *** | *** | **** | X | X |
| TCR | | | | | |
| Mock | **** | **** | **** | **** | X |
| D37244 | | | | | |
| IFN-γ | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ** | X | X | X | X |
| KO | | | | | |
| NR4A3 | ** | ns | X | X | X |
| KO | | | | | |
| Control | * | *** | *** | X | X |
| TCR | | | | | |
| Mock | **** | **** | **** | **** | X |
| D37244 | | | | | |
| IFN-γ | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | ** | *** | ** | X | X |
| TCR | | | | | |
| Mock | **** | **** | **** | **** | X |

ns-not significant,
*p < 0.05,
** p < 0.005,
*** p < 0.001,
**** p < 0.0001.

TABLE 4
Unpaired t-test statistical analysis of secreted interleukin-2 (IL-2) produced from NR4A-edited
and control non-edited NY-ESO-1 TCR T cells during the
A375 sequential stimulation assay corresponding to FIG. 15.

| D33018 | | | | | |
|---|---|---|---|---|---|
| IL-2 | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | * | X | X | X | X |
| KO | | | | | |
| NR4A3 | ** | * | X | X | X |
| KO | | | | | |
| Control | * | ns | * | X | X |
| TCR | | | | | |
| Mock | **** | **** | **** | **** | X |
| D33018 | | | | | |
| IL-2 | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ** | X | X | X | X |
| KO | | | | | |
| NR4A3 | * | ns | X | X | X |
| KO | | | | | |
| Control | ** | ns | ns | X | X |
| TCR | | | | | |
| Mock | *** | *** | ** | ** | X |
| D33018 | | | | | |
| IL-2 | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ns | X | X | X | X |
| KO | | | | | |
| NR4A3 | * | ns | X | X | X |
| KO | | | | | |
| Control | ns | ns | * | X | X |
| TCR | | | | | |
| Mock | ns | ns | * | ns | X |
| D33018 | | | | | |
| IL-2 | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | * | X | X | X | X |
| KO | | | | | |
| NR4A3 | ns | ns | X | X | X |
| KO | | | | | |
| Control | ns | * | ns | X | X |
| TCR | | | | | |
| Mock | ns | * | ns | ns | X |
| D35108 | | | | | |
| IL-2 | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ** | X | X | X | X |
| KO | | | | | |
| NR4A3 | ** | ns | X | X | X |
| KO | | | | | |
| Control | * | ns | ns | X | X |
| TCR | | | | | |
| Mock | *** | **** | **** | **** | X |
| D35108 | | | | | |
| IL-2 | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ** | X | X | X | X |
| KO | | | | | |
| NR4A3 | * | ** | X | X | X |
| KO | | | | | |
| Control | ** | ** | *** | X | X |
| TCR | | | | | |
| Mock | *** | **** | **** | **** | X |
| D35108 | | | | | |
| IL-2 | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ns | X | X | X | X |
| KO | | | | | |
| NR4A3 | * | **** | X | X | X |
| KO | | | | | |
| Control | ns | ** | **** | X | X |
| TCR | | | | | |
| Mock | ns | ** | **** | ns | X |
| D35108 | | | | | |
| IL-2 | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ns | X | X | X | X |
| KO | | | | | |
| NR4A3 | * | ** | X | X | X |
| KO | | | | | |
| Control | ns | * | ** | X | X |
| TCR | | | | | |
| Mock | * | ** | ** | * | X |
| D37244 | | | | | |
| IL-2 | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | ns | ** | ** | X | X |
| TCR | | | | | |
| Mock | ** | **** | **** | **** | X |
| D37244 | | | | | |
| IL-2 | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | * | * | ns | X | X |
| TCR | | | | | |
| Mock | *** | *** | ** | *** | X |
| D37244 | | | | | |
| IL-2 | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | * | X | X | X | X |
| KO | | | | | |
| NR4A3 | ** | ns | X | X | X |
| KO | | | | | |
| Control | * | *** | *** | X | X |
| TCR | | | | | |
| Mock | * | ** | ** | ** | X |
| D37244 | | | | | |
| IL-2 | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ** | X | X | X | X |
| KO | | | | | |
| NR4A3 | ** | ** | X | X | X |
| KO | | | | | |
| Control | ns | ** | ** | X | X |
| TCR | | | | | |
| Mock | ns | ** | *** | ns | X |

ns-not significant,
*p < 0.05,
** p < 0.005,
*** p < 0.001,
**** p < 0.0001.

TABLE 5
Unpaired t-test statistical analysis of secreted tumor necrosis factor alpha (TNF-α) produced
from NR4A-edited and control non-edited NY-ESO-1 TCR T cells
during the A375 sequential stimulation assay corresponding to FIG. 15.

| D33018 | | | | | |
|---|---|---|---|---|---|
| TNF-α | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | ns | ns | ** | X | X |
| TCR | | | | | |
| Mock | **** | **** | **** | **** | X |
| D33018 | | | | | |
| TNF-α | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ** | X | X | X |
| KO | | | | | |
| Control | ns | ns | ** | X | X |
| TCR | | | | | |
| Mock | *** | *** | *** | ** | X |
| D33018 | | | | | |
| TNF-α | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ns | X | X | X | X |
| KO | | | | | |
| NR4A3 | * | * | X | X | X |
| KO | | | | | |
| Control | ns | ns | * | X | X |
| TCR | | | | | |
| Mock | ** | *** | ** | ** | X |
| D33018 | | | | | |
| TNF-α | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | ns | ns | ns | X | X |
| TCR | | | | | |
| Mock | *** | ** | * | ** | X |
| D35108 | | | | | |
| TNF-α | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ns | X | X | X | X |
| KO | | | | | |
| NR4A3 | ** | * | X | X | X |
| KO | | | | | |
| Control | ns | ** | *** | X | X |
| TCR | | | | | |
| Mock | **** | **** | **** | **** | X |
| D35108 | | | | | |
| TNF-α | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | ** | X | X | X | X |
| KO | | | | | |
| NR4A3 | * | **** | X | X | X |
| KO | | | | | |
| Control | *** | *** | **** | X | X |
| TCR | | | | | |
| Mock | **** | **** | **** | **** | X |
| D35108 | | | | | |
| TNF-α | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ns | X | X | X | X |
| KO | | | | | |
| NR4A3 | ** | ns | X | X | X |
| KO | | | | | |
| Control | *** | ** | *** | X | X |
| TCR | | | | | |
| Mock | **** | ** * | **** | *** | X |
| D35108 | | | | | |
| TNF-α | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | ns | X | X | X | X |
| KO | | | | | |
| NR4A3 | ns | ns | X | X | X |
| KO | | | | | |
| Control | * | ns | * | X | X |
| TCR | | | | | |
| Mock | * | * | * | * | X |
| D37244 | | | | | |
| TNF-α | NR4A1 | NR4A2 | NR4A3 | Control | |
| Stim 1 | KO | KO | KO | TCR | Mock |
| NR4A1 | X | X | X | X | X |
| KO | | | | | |
| NR4A2 | ** | X | X | X | X |
| KO | | | | | |
| NR4A3 | * | * | X | X | X |
| KO | | | | | |
| Control | * | *** | *** | X | X |
| TCR | | | | | |
| Mock | *** | **** | **** | **** | X |
| D37244 | | | | | |
| TNF-α | NR4A1 | NR4A2 | NR4A3 | Control | |
| Stim 2 | KO | KO | KO | TCR | Mock |
| NR4A1 | X | X | X | X | X |
| KO | | | | | |
| NR4A2 | * | X | X | X | X |
| KO | | | | | |
| NR4A3 | * | ns | X | X | X |
| KO | | | | | |
| Control | *** | ** | ** | X | X |
| TCR | | | | | |
| Mock | **** | **** | **** | **** | X |
| D37244 | | | | | |
| TNF-α | NR4A1 | NR4A2 | NR4A3 | Control | |
| Stim 3 | KO | KO | KO | TCR | Mock |
| NR4A1 | X | X | X | X | X |
| KO | | | | | |
| NR4A2 | ** | X | X | X | X |
| KO | | | | | |
| NR4A3 | * | ns | X | X | X |
| KO | | | | | |
| Control | ** | *** | ** | X | X |
| TCR | | | | | |
| Mock | **** | *** | ** | *** | X |
| D37244 | | | | | |
| TNF-α | NR4A1 | NR4A2 | NR4A3 | Control | |
| Stim 4 | KO | KO | KO | TCR | Mock |
| NR4A1 | X | X | X | X | X |
| KO | | | | | |
| NR4A2 | * | X | X | X | X |
| KO | | | | | |
| NR4A3 | ** | ns | X | X | X |
| KO | | | | | |
| Control | * | ** | ** | X | X |
| TCR | | | | | |
| Mock | ** | ** | *** | * | X |

ns-not significant,
*p < 0.05,
** p < 0.005,
*** p < 0.001,
**** p < 0.0001.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

What is claimed is:

1. A method of treating a tumor in a subject in need thereof, comprising administering to the subject a cell composition comprising a population of modified immune cells that express reduced levels of Nuclear Receptor Subfamily 4 Group A Member 3 (“NR4A3”) gene and/or NR4A3 protein and a binding molecule and that have endogenous expression of NR4A1 and NR4A2 genes and NR4A1 and NR4A2 proteins.

2. The method of claim 1, wherein the expression levels of NR4A3 gene and/or NR4A3 protein in the population of modified immune cells is reduced by at least about 5%, at least about 10%, at least about 20%, at least about 30%, at least about 40%, at least about 50%, at least about 60%, at least about 70%, at least about 80%, at least about 90%, or at least about 100%, compared to a reference cell composition (e.g., corresponding cell composition wherein the cells have not been modified to express lower levels of NR4A3 gene and/or NR4A3 protein).

3. The method of claim 1 or 2, wherein the modified immune cells comprise lymphocytes, neutrophils, monocytes, macrophages, dendritic cells, and any combination thereof.

4. The method of claim 3, wherein the lymphocytes comprise T cells, tumor-infiltrating lymphocytes (TIL), lymphokine-activated killer cells, natural killer (NK) cells, and any combination thereof.

5. The method of claim 3, wherein the lymphocytes are T cells.

6. The method of any one of claims 1 to 5, wherein the binding molecule comprises a chimeric antigen receptor (CAR) and/or a T cell receptor (TCR), e.g., engineered TCR.

7. The method of any one of claims 1 to 6, wherein the binding molecule comprises a CAR.

8. The method of any one of claims 1 to 7, wherein the modified immune cells are ex vivo cells or in vitro cells.

9. The method of any one of claims 1 to 7, wherein the modified immune cells are in vivo cells.

10. The method of any one of claims 1 to 9, wherein the modified immune cells are modified by a gene editing tool to reduce the expression of the NR4A3 gene and/or NR4A3 protein.

11. The method of claim 10, wherein the gene editing tool comprises a shRNA, siRNA, miRNA, antisense oligonucleotides, CRISPR, zinc finger nuclease, TALEN, meganuclease, restriction endonuclease, or any combination thereof.

12. The method of claim 11, wherein the gene editing tool is CRISPR.

13. The method of any one of claims 1 to 12, wherein the population of modified immune cells exhibits one or more enhanced properties of the immune cells in the subject compared to the reference immune cells (i.e., immune cells that have not been modified to have reduced NR4A3 active gene levels and/or a reduced NR4A3 protein expression levels).

14. The method of claim 13, wherein the enhanced properties of the modified immune cell comprise (i) enhanced expansion of the immune cell, (ii) enhanced cytotoxicity of the immune cell, (iii) enhanced persistence, (iv) enhanced cytokine expression of the immune cell, or any combination thereof.

15. The method of claim 14, wherein the modified immune cells exhibit enhanced cytokine expression.

16. The method of claim 15, wherein the cytokines are Interleukin-2 (IL-2), Interferon-γ (IFN-γ), Tumor necrosis factor-α (TNF-α), or any combination thereof.

17. The method of claim 16, wherein the expression level of IL-2 is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of IL-2 in a population of reference immune cells.

18. The method of claim 16 or 17 wherein the expression level of IFN-γ is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of IL-γ in a population of reference immune cells.

19. The method of any one of claims 16 to 18, wherein the expression level of TNF-α is increased at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 4 fold, at least about 5 fold, at least about 6 fold, at least about 7 fold, at least about 8 fold, at least about 9 fold, at least about 10 fold compared to the expression level of TNF-α in a population of reference immune cells.

20. The method of any one of claims 1 to 19, wherein the modified immune cells exhibit reduced exhaustion or dysfunction compared to the reference immune cells do (i.e., immune cells that have not been modified to have reduced NR4A3 active gene levels and/or a reduced NR4A3 protein expression levels).

21. The method of claim 20, wherein the modified immune cells exhibit increased cytotoxicity upon sequential stimulation.

22. The method of claim 20, wherein the modified immune cells exhibit increased cytotoxicity in chronic stimulation.

23. The method of any one of claims 1 to 22, wherein the modified immune cells maintain an anti-tumor function in tumor microenvironment (TME).

24. The method of any one of claims 1 to 23, wherein the binding molecule comprises an scFv derived from R12, R11, 2A2, or any combination thereof.

25. The method of any one of claims 1 to 24, wherein the binding molecule comprises a heavy chain variable domain comprising SEQ ID NO: 17 and a light chain variable domain comprising SEQ ID NO: 21.

26. The method of any one of claim 1 to 25, wherein the administering reduces a tumor volume in the subject, compared to a reference tumor volume (e.g., tumor volume in the subject prior to the administration and/or tumor volume in a subject that did not receive the administration).

27. The method of claim 26, wherein the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor volume (e.g., the tumor volume in the subject prior to the administration and/or tumor volume in a subject that did not receive the administration).

28. The method of any one of claims 1 to 27, wherein the administering reduces a tumor weight in the subject, compared to a reference tumor weight (e.g., tumor weight in the subject prior to the administration and/or tumor weight in a subject that did not receive the administration).

29. The method of claim 28, wherein the tumor weight is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor weight (e.g., tumor weight in the subject prior to the administration and/or tumor weight in a subject that did not receive the administration).

30. The method of any one of claims 1 to 29, wherein the administering increases the duration of survival of the subject as compared to a reference duration of survival (e.g., duration of survival in the subject prior to the administration and/or duration of survival in a subject that did not receive the administration).

31. The method of claim 30, wherein, compared to the reference duration of survival, the duration of survival is increased by at least about one week, at least about two weeks, at least about three weeks, at least about one month, at least about two months, at least about three months, at least about four months, at least about five months, at least about six months, at least about seven months, at least about eight months, at least about nine months, at least about 10 months, at least about 11 months, or at least about one year.

32. The method of any one of claims 1 to 31, wherein the administering reduces or prevents exhaustion or dysfunction of the immune cells.

33. The method of any one of claims 1 to 32 wherein the immune cells maintain an anti-tumor function in tumor microenvironment (TME).

34. The method of any one of claims 1 to 33, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, cervical cancer, carcinoma, sarcoma, leukemia, lymphoma, myeloma, or a combination thereof.

35. The method of any one of claims 1 to 34, comprising administering an additional therapeutic agent to the subject.

36. The method of claim 35, wherein the additional therapeutic agent comprises a chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

37. The method of claim 36, wherein the additional therapeutic agent is an immune checkpoint inhibitor.

38. The method of claim 36 or 37, wherein the immune checkpoint inhibitor comprises an anti-PD-1 antibody, an anti-PD-L1 antibody, an anti-LAG-3 antibody, an anti-CTLA-4 antibody, an anti-GITR antibody, an anti-TIM3 antibody, and any combination thereof.

39. The method of any one of claims 35 to 38, wherein the additional therapeutic agent and the cell composition are administered concurrently.

40. The method of any one of claims 35 to 38, wherein the additional therapeutic agent and the cell composition are administered sequentially.

41. The method of any one of claims 1 to 40, wherein the cell composition is administered parenterally, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

42. The method of any one of claims 10 to 41, wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

43. A method of generating a cell having a reduced level of Nuclear Receptor Subfamily 4 Group A Member 3 “(NR4A3”) gene and/or NR4A3 protein, comprising modifying the cells with a gene editing tool, wherein the gene editing tool reduces the expression of the NR4A3 gene and/or NR4A3 protein, wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

44. A method of increasing the production of a cytokine by immune cells in response to an antigen stimulation, comprising modifying the immune cells with a gene editing tool, wherein the gene editing tool reduces the expression of the NR4A3 gene and/or NR4A3 protein, and wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

45. The method of claim 44, wherein the cytokine comprises IFN-γ, IL-2, TNF-α, or a combination thereof.

46. The method of claim 44 or 45, wherein, after the modification, the production of the cytokine in response to the antigen stimulation is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, or at least about 50-fold, compared to corresponding immune cells that were not modified with the gene editing tool.

47. A method of increasing an effector function of immune cells in response to persistent antigen stimulation, comprising modifying the immune cells with a gene editing tool, wherein the gene editing tool reduces the expression of the NR4A3 gene and/or NR4A3 protein, and wherein the gene editing tool comprises a guide RNA comprising, consisting of, or consisting essentially of the sequence set forth in any one of SEQ ID NO: 30, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

48. The method of claim 47, wherein the immune cells retain effector function for at least one, at least two, or at least three additional rounds of an antigen stimulation assay, as compared to reference immune cells.

49. The method of claim 47 or 48, wherein the effector function comprises the ability: (i) to kill target cells (e.g., tumor cells) (ii) to produce a cytokine upon further antigen stimulation, or (iii) both (i) and (ii).

50. The method any one of claims 42 to 49, wherein the guide RNA comprises, consists of, or consists essentially of the sequence set forth in SEQ ID NO: 30.

51. The method any one of claims 42 to 49, wherein the guide RNA comprises, consists of, or consists essentially of the sequence set forth in SEQ ID NO: 52.

52. The method any one of claims 42 to 49, wherein the guide RNA comprises, consists of, or consists essentially of the sequence set forth in SEQ ID NO: 53.

53. The method any one of claims 42 to 49, wherein the guide RNA comprises, consists of, or consists essentially of the sequence set forth in SEQ ID NO: 54.

54. The method any one of claims 42 to 49, wherein the guide RNA comprises, consists of, or consists essentially of the sequence set forth in SEQ ID NO: 55.

55. The method any one of claims 42 to 49, wherein the guide RNA comprises, consists of, or consists essentially of the sequence set forth in SEQ ID NO: 56.

56. The method any one of claims 42 to 49, wherein the guide RNA comprises, consists of, or consists essentially of the sequence set forth in SEQ ID NO: 57.

57. The method any one of claims 42 to 49, wherein the guide RNA comprises, consists of, or consists essentially of the sequence set forth in SEQ ID NO: 58.

58. The method any one of claims 42 to 49, wherein the guide RNA comprises, consists of, or consists essentially of the sequence set forth in SEQ ID NO: 61.

59. The method any one of claims 42 to 49, wherein the guide RNA comprises, consists of, or consists essentially of the sequence set forth in SEQ ID NO: 65.

60. The method any one of claims 42 to 49, wherein the guide RNA comprises, consists of, or consists essentially of the sequence set forth in SEQ ID NO: 67.

61. The method any one of claims 42 to 49, wherein the guide RNA comprises, consists of, or consists essentially of the sequence set forth in SEQ ID NO: 68.

62. The method any one of claims 42 to 49, wherein the guide RNA comprises, consists of, or consists essentially of the sequence set forth in SEQ ID NO: 70.

63. The method any one of claims 42 to 49, wherein the guide RNA comprises, consists of, or consists essentially of the sequence set forth in SEQ ID NO: 71.

64. The method any one of claims 42 to 49, wherein the guide RNA comprises, consists of, or consists essentially of the sequence set forth in SEQ ID NO: 75.

65. The method any one of claims 42 to 49, wherein the guide RNA comprises, consists of, or consists essentially of the sequence set forth in SEQ ID NO: 76.

66. The method any one of claims 42 to 49, wherein the guide RNA comprises, consists of, or consists essentially of the sequence set forth in SEQ ID NO: 82.

67. The method any one of claims 42 to 49, wherein the guide RNA comprises, consists of, or consists essentially of the sequence set forth in SEQ ID NO: 83.

68. The method any one of claims 42 to 49, wherein the guide RNA comprises, consists of, or consists essentially of the sequence set forth in SEQ ID NO: 86.

69. The method any one of claims 42 to 49, wherein the guide RNA comprises, consists of, or consists essentially of the sequence set forth in SEQ ID NO: 94.

70. The method any one of claims 42 to 49, wherein the guide RNA comprises, consists of, or consists essentially of the sequence set forth in SEQ ID NO: 96.

71. A composition comprising a cell, wherein the cell has been prepared by the method of claim 43.

72. A composition comprising a cell which expresses a reduced level of a NR4A3 gene and/or NR4A3 protein, wherein the cell has been modified with a gRNA that can target the NR4A3 gene, wherein the gRNA comprises, consists of, or consists essentially of the sequence set forth in any one of SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 61, SEQ ID NO: 65, SEQ ID NO: 67, SEQ ID NO: 68, SEQ ID NO: 70, SEQ ID NO: 71, SEQ ID NO: 75, SEQ ID NO: 76, SEQ ID NO: 82, SEQ ID NO: 83, SEQ ID NO: 86, SEQ ID NO: 94, and SEQ ID NO: 96.

73. The composition of claim 72, wherein the gRNA comprises, consists of, or consists essentially of the sequence set forth in SEQ ID NO: 30.

74. The composition of claim 72, wherein the gRNA comprises, consists of, or consists essentially of the sequence set forth in SEQ ID NO: 52.

75. The composition of claim 72, wherein the gRNA comprises, consists of, or consists essentially of the sequence set forth in SEQ ID NO: 53.

76. The composition of claim 72, wherein the gRNA comprises, consists of, or consists essentially of the sequence set forth in SEQ ID NO: 54.

77. The composition of claim 72, wherein the gRNA comprises, consists of, or consists essentially of the sequence set forth in SEQ ID NO: 55.

78. The composition of claim 72, wherein the gRNA comprises, consists of, or consists essentially of the sequence set forth in SEQ ID NO: 56.

79. The composition of claim 72, wherein the gRNA comprises, consists of, or consists essentially of the sequence set forth in SEQ ID NO: 57.

80. The composition of claim 72, wherein the gRNA comprises, consists of, or consists essentially of the sequence set forth in SEQ ID NO: 58.

81. The composition of claim 72, wherein the gRNA comprises, consists of, or consists essentially of the sequence set forth in SEQ ID NO: 61.

82. The composition of claim 72, wherein the gRNA comprises, consists of, or consists essentially of the sequence set forth in SEQ ID NO: 65.

83. The composition of claim 72, wherein the gRNA comprises, consists of, or consists essentially of the sequence set forth in SEQ ID NO: 67.

84. The composition of claim 72, wherein the gRNA comprises, consists of, or consists essentially of the sequence set forth in SEQ ID NO: 68.

85. The composition of claim 72, wherein the gRNA comprises, consists of, or consists essentially of the sequence set forth in SEQ ID NO: 70.

86. The composition of claim 72, wherein the gRNA comprises, consists of, or consists essentially of the sequence set forth in SEQ ID NO: 71.

87. The composition of claim 72, wherein the gRNA comprises, consists of, or consists essentially of the sequence set forth in SEQ ID NO: 75.

88. The composition of claim 72, wherein the gRNA comprises, consists of, or consists essentially of the sequence set forth in SEQ ID NO: 76.

89. The composition of claim 72, wherein the gRNA comprises, consists of, or consists essentially of the sequence set forth in SEQ ID NO: 82.

90. The composition of claim 72, wherein the gRNA comprises, consists of, or consists essentially of the sequence set forth in SEQ ID NO: 83.

91. The composition of claim 72, wherein the gRNA comprises, consists of, or consists essentially of the sequence set forth in SEQ ID NO: 86.

92. The composition of claim 72, wherein the gRNA comprises, consists of, or consists essentially of the sequence set forth in SEQ ID NO: 94.

93. The composition of claim 72, wherein the gRNA comprises, consists of, or consists essentially of the sequence set forth in SEQ ID NO: 96.

94. The composition of any one of claims 71 to 93, wherein the cells comprise in vivo cells.

95. The cell composition of any one of claims 71 to 93, wherein the cells comprise ex vivo cells or in vitro cells.

96. A pharmaceutical composition comprising the cell composition of any one of claims 71 to 95.

97. A kit comprising (i) a gene editing tool to reduce the expression of NR4A3 gene and/or NR4A3 protein, (ii) a vector comprising a chimeric antigen receptor (CAR) or a T cell receptor (TCR), and instructions for treating tumor according to the method of any one of claims 1 to 42.

98. A kit comprising (i) a gene editing tool to reduce the expression of NR4A3 gene and/or NR4A3 protein, (ii) a vector comprising a chimeric antigen receptor (CAR) or a T cell receptor (TCR), and instructions for preparing a composition comprising a population of cells, wherein the cells are prepared according to the method of claim 43.
